(19) United States
(12) Patent Application Publication
(10) Pub. No.: US 2025/0179510 A1
(43) Pub. Date: Jun. 5, 2025

(54) METHODS AND MEANS FOR GENOME EDITING USING GUIDE RNAS EXPRESSED UNDER CONTROL OF DIFFERENT PROMOTERS

(71) Applicant: Monsanto Technology LLC, St. Louis, MO (US)

(72) Inventors: Ian W. Davis, Grover, MO (US); Changlin Fu, Chesterfield, MO (US); Matthew S. Marengo, Wildwood, MO (US); Ervin D. Nagy, Lake St. Louis, MO (US); Chenxi Wu, Ballwin, MO (US)

(21) Appl. No.: 18/965,138

(22) Filed: Dec. 2, 2024

Related U.S. Application Data
(60) Provisional application No. 63/605,956, filed on Dec. 4, 2023.

(51) Int. Cl. C12N 15/82; C12N 9/22; C12N 15/11

(52) U.S. Cl. CPC C12N 15/8213; C12N 9/22; C12N 15/11; C12N 2310/20

(57) ABSTRACT

Compositions and methods are provided for the expression of similar guide nucleic acids for guided nucleases from multiple recombinant cassettes under control of different promoters in cells.

### CROSS-REFERENCE TO RELATED APPLICATIONS

This application claims priority of U.S. Provisional Application Ser. No. 63/605,956 filed on Dec. 4, 2023, the entire disclosure of which is incorporated herein by reference.

### FIELD

The present disclosure relates to compositions and methods related to expression of guide nucleic acids for guided nucleases from multiple recombinant cassettes under control of different promoters in cells.

### INCORPORATION OF SEQUENCE LISTING

A sequence listing contained in the file named “MONS587US_anniv_ST26.xml” which is 74,242 bytes (measured in MS-Windows®) and was created on Nov. 29, 2024, containing 49 sequences, is filed electronically herewith and incorporated by reference in its entirety.

### BACKGROUND OF THE INVENTION

Genome editing, using RNA guided nucleases, such as CRISPR/Cas proteins, holds great promise for medicines and agricultural applications. While some forms of genome editing, such as targeted indels, are of sufficient efficacy in most eukaryotic organisms, other forms, such as site-directed integration (SDI) of exogenous gene cassettes or precise edits by homology directed repair (HDR) or by alternative technologies, such as base editing (BE) or prime editing (PE), are still lacking in efficacy. Multiple lines of evidence indicate that these challenging forms of edits are in strong correlation with the efficacy of the core capabilities of the CRISPR machinery, i.e. invasion and cleavage of the chromosomal DNA in a sequence-specific manner. Guide RNAs can be designed in a straightforward way by identifying a sequence in the genome to be edited corresponding to a protospacer adjacent motif (PAM) for the CAS effector to be used and design of a nucleotide sequence having homology to the genomic nucleotide sequence located 5′ or 3′ of the PAM (depending on the CAS effector to be used) as a seed sequence for the guide RNAs. However, not all guide RNAs exhibit the same cutting and/or editing efficiency, thus limiting the applicability of guide RNAs in genomic regions where potential guide RNAs are not or less frequently available.

The present invention addresses these shortcomings in the art by providing new compositions and methods for expressing guide nucleic acids and guide RNAs for RNA guided nucleases such as CRISPR/Cas proteins in cells, such as eukaryotic cells.

### SUMMARY OF THE INVENTION

In summary, the invention is directed at the following numbered embodiments:

Embodiment 1: A method for editing the genome of a cell at a target site in the cell comprising the steps of
a. providing an RNA guided effector protein, or a nucleic acid encoding the RNA guided effector protein, to the cell; and
b. providing a guide RNA recognizing the target site to the cell, wherein the guide RNA is expressed in the cell from at least two recombinant cassettes;
wherein each of the recombinant cassettes comprises a promoter operably linked to a nucleic acid encoding the guide RNA and wherein the promoter of each recombinant cassette is different from the promoter of the other recombinant cassette or cassettes expressing the guide RNA.

Embodiment 2: A method for increasing editing at a target site in the genome of a cell which is moderately or poorly recognized by a guide RNA, comprising the steps of:
a. providing the cell with at least two guide RNAs wherein each of the guide RNAs recognizes the target site, and wherein the guide RNAs are expressed in the cell under control of different promoters; and
b. providing an RNA guided effector protein or a nucleic acid encoding the RNA guided effector protein to the cell;
wherein the editing efficiency at the target site is increased when compared to editing in the genome of a cell comprising an RNA guided endonuclease and a guide RNA expressed from a single recombinant construct under control of a single expressible promoter.

Embodiment 3: A method for editing the genome of a cell at a target site in the cell comprising the steps of
a. introducing at least two recombinant cassettes in the cell encoding the same or similar guide RNA recognizing the target site, and wherein the recombinant cassettes comprise different promoters, to produce a cell comprising the recombinant cassettes; and
b. introducing an RNA guided effector protein, or a nucleic acid encoding the RNA guided effector protein, into the cell comprising the recombinant cassettes encoding the same or similar guide RNA recognizing the target site.

Embodiment 4: A method for editing the genome of a cell at a target site in the cell comprising the step of introducing an RNA guided effector protein, or a nucleic acid encoding the RNA guided effector protein, into a cell comprising at least two recombinant cassettes encoding the same or similar guide RNA recognizing the target site, and wherein the guide RNA is expressed in the cell under control of different promoters.

Embodiment 5: A method for editing the genome of a cell at a target site in the cell comprising the step of introducing at least two recombinant cassettes encoding the same or similar guide RNA recognizing the target site, and wherein the guide RNA is expressed in the cell under control of different promoters, into a cell comprising the target site, the cell further comprising an RNA guided effector protein or a nucleic acid encoding the RNA guided effector protein.

Embodiment 6: A method for editing the genome of a cell at multiple target sites in the cell comprising the steps of:
a. providing the cell with one or more RNA guided effector proteins, or one or more nucleic acids encoding the RNA guided effector proteins; and
b. providing the cells with multiple guide RNAs, each guide RNA recognizing one of the multiple target sites,
c. wherein each guide RNA recognizing one of the multiple target sites is expressed from a multitude of recombinant cassettes, each recombinant cassette comprising a promoter, and wherein the promoters of each recombinant cassette encoding the same or similar guide RNA are different.

Embodiment 7: The method according to any one of embodiments 1 to 6, wherein the RNA guided effector protein is a CRISPR-Cas effector protein, selected from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system, or a CRISPR-Cas effector protein derived therefrom, optionally a CRISPR-Cas effector protein comprising one or more nuclear localization signals.

Embodiment 8: The method according to any one of embodiments 1 to 7 wherein the RNA guided effector protein is a fusion protein comprising a cleavage domain, a nuclease domain, a deaminase domain, a cytosine deaminase domain, an adenine deaminase domain, a transcription activator domain, a transcription repression domain, a reverse transcriptase domain, a uracil DNA glycolase inhibitor, a Dna2 polypeptide, and/or a 5′ flap endonuclease.

Embodiment 9: The method according to any one of embodiments 1 to 7, wherein the RNA guided endonuclease is a CRISPR-Cas effector protein selected from a Cas9, C2c1, C2c3, Cas12a (also referred to as Cpf1), Cas12b, Cas12c, Cas12d, Cas12c, Cas13a, Cas13b, Cas13c, Cas13d, Cas1, CaslB, Cas2, Cas3, Cas3′, Cas3″, Cas4, Cas5, Cas6, Cas7, Cas8, Csn1, Csx12, Cas10, Csy1, Csy2, Csy3, Cse1, Cse2, 30 Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), Csf5 nuclease, Cas12c (C2c3), Cas12d (CasY), Cas12c (CasX), Cas12g, Cas12h, Cas12i, C2c4, C2c5, C2c8, C2c9, C2c10, Cas14a, Cas14b, Cas14c effector protein.

Embodiment 10: The method according to any one of embodiments 1 to 9, wherein the RNA guided effector protein is a Cas12a effector protein or a Cas12a derived effector protein.

Embodiment 11: The method according to embodiment 10, wherein the Cas12a effector protein is selected from FnCas12a, LbCas12a, ErCas12a (MAD7®) or AsCas12a or variants thereof.

Embodiment 12: The method according to any one of embodiments 1 to 11, wherein the RNA guided effector protein is provided to, or introduced into the cell by expression from a recombinant cassette comprising a nucleic acid encoding the RNA guided effector protein, operably linked to a plant-expressible promoter.

Embodiment 13: The method according to embodiment 12, wherein the RNA guided effector protein is a Cas12a effector protein or Cas12a derived effector protein selected from FnCas12a, LbCas12a, ErCas12a (MAD7) or AsCas12a or variants thereof.

Embodiment 14: The method according to embodiment 12 or embodiment 13, wherein nucleic acid fragment encoding the RNA guided effector protein comprises is a nucleotide sequence encoding the RNA guided effector protein which is codon-optimized for expression in the cell, such as a plant-codon optimized nucleotide sequence.

Embodiment 15: The method according to any one of embodiments 12 to 14 wherein the RNA guided effector protein comprises an amino acid sequence having at least 90% or 95% sequence identity to an amino acid sequence selected from any one of SEQ ID NOs: 4-6.

Embodiment 16: The method according to embodiment 12, wherein the nucleic acid fragment encoding the RNA guided effector protein comprises a nucleotide sequence of SEQ ID NO 2.

Embodiment 17: The method according to any one of embodiments 12 to 16, wherein the plant expressible promoter expressing the RNA guide effector protein is selected from constitutive, inducible, temporally regulated, developmentally regulated, chemically regulated, tissue-preferred and/or tissue-specific promoters or is selected from a meiotic promoter, an egg cell-preferred or embryo-tissue preferred promoter such as a DSULI promoter, an EAl promoter, an ES4 promoter, a DMC1 promoter, a Mps1 promoter, an Adf1 promoter or an EAL promoter, or is a floral-tissue preferred or floral cell-preferred promoter.

Embodiment 18: The method according to any one of embodiments 1 to 17, wherein the promoter expressing the guide RNA is a plant-expressible promoter.

Embodiment 19: The method according to any one of embodiments 1 to 17, wherein the promoter(s) expressing the guide RNA(s) is/are DNA dependent RNA Polymerase III promoter(s) (Pol III promoter).

Embodiment 20: The method according to embodiment 19, wherein the Pol III promoters are selected from the group consisting of a U6 promoter, an H1 promoter, a 5S promoter, an Adenovirus 2 (Ad2) VAI promoter, a tRNA promoter, a 7SK promoter, chimeric Pol III promoters or synthetic Pol III promoters.

Embodiment 21: The method according to any one of embodiments 19 or 20, wherein the Pol III promoters comprise a nucleotide sequence selected from any one of SEQ ID NOs: 10-13, 15-16 or 18-45.

Embodiment 22: The method according to any one of embodiments 1 to 21, wherein the cell is a eukaryotic cell.

Embodiment 23: The method according to embodiments 22, wherein the cell is a plant cell.

Embodiment 24: The method according to embodiment 23, wherein the plant cell is from a plant selected from a monocotyledonous species, a dicotyledonous species, an angiosperm species or a gymnosperm species.

Embodiment 25: The method according to embodiment 23 or 24 wherein the plant cell is from a plant selected from a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, or a potato plant.

Embodiment 26: The method according to any one of embodiments 1 to 25, wherein the guide RNA is a single guide RNA.

Embodiment 27: The method according to any one of embodiments 1 to 26, wherein the guide RNA comprises two direct repeat sequences, flanking a spacer comprising a nucleotide sequence complementary to the target site.

Embodiment 28: The method according to any one of embodiments 1 to 27, wherein the guide RNA is a guide RNA which results in moderate or poor editing or has a moderate or low editing efficiency at the target site, optionally a guide RNA which results in a cutting efficiency of less than 20%.

Embodiment 29: The method according to any one of embodiments 1 to 28, wherein the guide RNA is a guide RNA with an editing efficiency score, as defined herein, which is lower than editing efficiency scores for other guide RNAs targeting the same target sequence in the genome of a plant.

Embodiment 30: The method according to embodiment 29, wherein the guide RNA has an editing efficiency score of less than 1, between 0 and 1 or less than 0.

Embodiment 31: The method according to any one of embodiments 1 to 30, wherein the editing comprises
a. inserting at least one nucleotide;
b. deleting at least one nucleotide;
c. substituting at least one nucleotide.

Embodiment 32: The method according to any one of embodiments 1 to 31, further comprising introducing or providing a donor template into the cell.

Embodiment 33: The method according to any one of embodiments 1 to 32, further comprising a step of regenerating a plant from the cell.

Embodiment 34: A cell comprising at least two recombinant cassettes, each cassette expressing a guide RNA recognizing a target site in the genome of the cell under control of a promoter, wherein the guide RNA is the same or similar and recognizes the same target site, and wherein the promoter of each cassette is different from the promoter of the other recombinant cassette or cassettes.

Embodiment 35: A cell according to embodiment 34, wherein the promoter(s) expressing the guide RNA is/are (a) plant-expressible promoter(s).

Embodiment 36: The cell according to any one of embodiments 34 or 35, wherein the promoter(s) expressing the guide RNA(s) is/are (a) Pol III promoter(s).

Embodiment 37: The cell according to embodiment 36, wherein the Pol III promoters are selected from the group consisting of a U6 promoter, an H1 promoter, a 5S promoter, an Adenovirus 2 (Ad2) VAI promoter, a tRNA promoter, a 7SK promoter, chimeric Pol III promoters or synthetic Pol III promoters.

Embodiment 38: The cell according to any one of embodiments 36 or 37, wherein the Pol III promoters comprise a nucleotide sequence selected from any one of SEQ ID NOs: 10-13, 15-16 or 18-45.

Embodiment 39: A cell according to any one of embodiments 35 to 38 further comprising an RNA guided effector protein, or a nucleic acid encoding the RNA guided effector as described in any one of embodiments 7 to 18.

Embodiment 40: The cell according to any one of embodiments 34 to 39, wherein the guide RNA is a single guide RNA.

Embodiment 41: The cell according to any one of embodiments 34 to 40, wherein the guide RNA comprises two direct repeat sequences, flanking a spacer comprising a nucleotide sequence complementary to the target site.

Embodiment 42: The cell according to any one of embodiments 34 to 41, wherein the guide RNA is a guide RNA which results in moderate or poor editing or has a moderate or low editing efficiency at the target site, optionally a guide RNA which results in a cutting efficiency of less than 20%.

Embodiment 43: The cell according to any one of embodiments 34 to 42, wherein the guide RNA is a guide RNA with an editing efficiency score, as defined herein, which is lower than editing efficiency scores for other guide RNAs targeting the same target sequence in the genome of a plant.

Embodiment 44: The cell according to embodiment 43, wherein the guide RNA has an editing efficiency score of less than 1, between 0 and 1 or less than 0.

Embodiment 45: A cell according to any one of claims 34 to 44, wherein the cell is a eukaryotic cell.

Embodiment 46: The cell according to embodiment 45, wherein the cell is a plant cell.

Embodiment 47: The cell according to embodiment 46, wherein the plant cell is from a plant selected from a monocotyledonous species, a dicotyledonous species, an angiosperm species or a gymnosperm species.

Embodiment 48: The cell according to embodiment 45 or 46, wherein the plant cell is from a plant selected from a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, or a potato plant.

Embodiment 49: A plant comprising a cell, or consisting essentially of cells, according to any one of embodiments 46 to 48.

Embodiment 50: A vector comprising at least two recombinant cassettes, each cassette expressing a guide RNA recognizing a target site in the genome of the cell under control of a promoter, wherein the guide RNA is the same or similar and recognizes the same target site, and wherein the promoter of each cassette is different from the promoter of the other recombinant cassette or cassettes.

Embodiment 51: The vector according to embodiment 50, wherein the promoter(s) expressing the guide RNA is/are (a) plant-expressible promoter.

Embodiment 52: The vector according to embodiment 50 or claim 51, wherein the promoter(s) expressing the guide RNA(s) is/are (a) Pol III promoter(s).

Embodiment 53: The vector according to embodiment 52, wherein the Pol III promoters are selected from the group consisting of a U6 promoter, an H1 promoter, a 5S promoter, an Adenovirus 2 (Ad2) VAI promoter, a tRNA promoter, a 7SK promoter, chimeric Pol III promoters or synthetic Pol III promoters.

Embodiment 54: The vector according to any one of embodiment 52 or embodiment 53, wherein the Pol III promoters comprise a nucleotide sequence selected from any one of SEQ ID NOs: 10-13, 15-16 or 18-45.

Embodiment 55: The vector according to any one of embodiments 50 to 54, further comprising an RNA guided effector protein, or a nucleic acid encoding the RNA guided effector proteins, as described in any one of claims 7 to 18.

Embodiment 56: The vector according to any one of embodiments 50 to 55, wherein the guide RNA is a single guide RNA.

Embodiment 57: The vector according to any one of embodiments 50 to 56, wherein the guide RNA comprises two direct repeat sequences, flanking a spacer comprising a nucleotide sequence complementary to the target site.

Embodiment 58: The vector according to any one of embodiments 50 to 57, wherein the guide RNA is a guide RNA which results in moderate or poor editing or has a moderate or low editing efficiency at the target site, optionally a guide RNA which results in a cutting efficiency of less than 20%.

Embodiment 59: The vector according to any one of embodiments 50 to 58, wherein the guide RNA is a guide RNA with an editing efficiency score, as defined herein, which is lower than editing efficiency scores for other guide RNAs targeting the same target sequence in the genome of a plant.

Embodiment 60: The vector according to embodiment 59, wherein the guide RNA has an editing efficiency score of less than 1, between 0 and 1 or less than 0.

Embodiment 61: A set of at least two recombinant cassettes, each cassette expressing a guide RNA recognizing a target site in the genome of the cell under control of a promoter, wherein the guide RNA is the same or similar and recognizes the same target site, and wherein the promoter of each cassette is different from the promoter of the other recombinant cassette or cassettes.

Embodiment 62: The set of at least two recombinant cassettes according to embodiment 61, wherein the promoter(s) expressing the guide RNA is/are (a) plant-expressible promoter.

Embodiment 63: The set of at least two recombinant cassettes according to embodiment 61 or embodiment 62, wherein the promoter(s) expressing the guide RNA(s) is/are (a) Pol III promoter(s).

Embodiment 64: The set of at least two recombinant cassettes according to embodiment 63, wherein the Pol III promoters are selected from the group consisting of a U6 promoter, an H1 promoter, a 5S promoter, an Adenovirus 2 (Ad2) VAI promoter, a tRNA promoter, a 7SK promoter, chimeric Pol III promoters or synthetic Pol III promoters.

Embodiment 65: The set of at least two recombinant cassettes according to any one of embodiment 63 or embodiment 64, wherein the Pol III promoters comprise a nucleotide sequence selected from any one of SEQ ID NOs: 10-13, 15-16 or 18-45.

Embodiment 66: The set of at least two recombinant cassettes according to any one of embodiments 61 to 65, further comprising an RNA guided effector protein, or a nucleic acid encoding the RNA guided effector proteins, as described in any one of embodiments 7 to 18.

Embodiment 67: The set of at least two recombinant cassettes according to any one of embodiments 61 to 66, wherein the guide RNA is a single guide RNA.

Embodiment 68: The set of at least two recombinant cassettes according to any one of embodiments 61 to 67, wherein the guide RNA comprises two direct repeat sequences, flanking a spacer comprising a nucleotide sequence complementary to the target site.

Embodiment 69: The set of at least two recombinant cassettes according to any one of embodiments 61 to 68, wherein the guide RNA is a guide RNA which results in moderate or poor editing or has a moderate or low editing efficiency at the target site, optionally a guide RNA which results in a cutting efficiency of less than 20%.

Embodiment 70: The set of at least two recombinant cassettes according to any one of embodiments 61 to 69, wherein the guide RNA is a guide RNA with an editing efficiency score, as defined herein, which is lower than editing efficiency scores for other guide RNAs targeting the same target sequence in the genome of a plant.

Embodiment 71: The set of at least two recombinant cassettes according to embodiment 69, wherein the guide RNA has an editing efficiency score of less than 1, between 0 and 1 or less than 0.

Embodiment 72: A cell comprising a vector according to any one of embodiments 50 to 60 or a set of at least two recombinant cassettes according to any one of embodiments 61 to 71.

Embodiment 73: The cell of claim 72, further comprising an RNA guided effector protein, or a nucleic acid encoding the RNA guided effector protein, as described in any one of embodiments 7 to 18.

Embodiment 74: The cell of embodiment 72 or 73, wherein the vector or the set of at least two recombinant cassettes is transiently introduced or wherein the vector or the set of at least two recombinant cassettes is stably integrated in the cell's genome.

Embodiment 75: The cell of embodiment 74, wherein the vector or the set of at least two recombinant cassettes is transiently introduced or wherein the vector is stably integrated in the cell's genome or wherein the nucleic acid encoding the RNA guided effector protein is transiently introduced in the cell, or wherein the nucleic acid encoding the RNA guided effector protein is stably integrated in the cell's genome, or wherein the vector or the set of at least two recombinant cassettes and the nucleic acid encoding the RNA guided effector protein are both stably integrated in the cell's genome.

Embodiment 76: The cell according to any one of embodiments 72 to 75, wherein the cell is a eukaryotic cell.

Embodiment 77: The cell according to embodiments 76, wherein cell is a plant cell.

Embodiment 78: The cell according to embodiment 77, wherein the plant cell is from a plant selected from a monocotyledonous species, a dicotyledonous species, an angiosperm species or a gymnosperm species.

Embodiment 79: The cell according to embodiment 77 or 78, wherein the plant cell is from a plant selected from a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, or a potato plant.

Embodiment 80: A plant comprising a cell or consisting essentially of cells according to any one of embodiments 77 to 79.

Embodiment 81: A plant comprising at set of at least two recombinant cassettes according to any one of embodiments 61 to 71.

Embodiment 82: The plant according to embodiment 81, further comprising a an RNA guided effector protein, or a nucleic acid encoding the RNA guided effector protein, as described in any one of embodiments 7 to 18.

Embodiment 83: A method for editing the genome of a cell at a target site in the cell comprising the steps of
a. providing one or more RNA guided effector proteins or a nucleic acid encoding the RNA guided effector proteins as described in any one of embodiments 7 to 18, to the cell comprising the target site; and
b. providing a vector according to any one of embodiments 50 to 60 or a set of at least two recombinant cassettes according to any one of embodiments 61 to 71 to the cell; or
c. providing one or more RNA guided effector proteins or a nucleic acid encoding the RNA guided effector proteins as described in any one of embodiments 7 to 18 to the cell comprising the target site, the cell further comprising a vector according to any one of embodiments 50 to 60 or further comprising a set of at least two recombinant cassettes according to any one of embodiments 61 to 71; or
d. providing a vector according to any one of embodiments 50 to 60 or a set of at least two recombinant cassettes according to any one of embodiments 61 to 71 to a cell comprising the target site, the cell further comprising one or more RNA guided effector proteins or a nucleic acid encoding the one or more RNA guided effector proteins.

Embodiment 84: The method according to embodiment 83, wherein the cell is a eukaryotic cell.

Embodiment 85: The method according to embodiment 84, wherein the cell is a plant cell.

Embodiment 86: The method according to embodiment 85, further comprising a step of regenerating the cell into a plant.

Embodiment 87: The method according to embodiment 83, wherein the providing is achieved by transient or stable transformation.

Embodiment 88: The method according to embodiment 83, wherein the cell is in a plant and the providing is achieved by crossing of plants comprising the nucleic acid encoding the RNA guided effector protein and plants comprising the vector or the set of at least two recombinant cassettes.

Embodiment 89: A cell obtainable by the method according to embodiment 83, wherein the cell comprises an edit at or near the target site the edit comprising insertion of at least one nucleotide, deletion of one nucleotide or substitution of at least one nucleotide.

Embodiment 90: A plant obtainable by the method of embodiment 86, wherein the plant comprises a cell having an edit at or near the target site the edit comprising insertion of at least one nucleotide, deletion of one nucleotide or substitution of at least one nucleotide, optionally wherein the plant consists essentially of cells having an edit at or near the target site the edit comprising insertion of at least one nucleotide, deletion of one nucleotide or substitution of at least one nucleotide.

Embodiment 91: A method for editing the genome of a plant at a target site in the plant genome comprising the steps of
a. providing a first plant comprising the target site and comprising a nucleic acid encoding the RNA guided effector proteins as described in any one of embodiments 7 to 18; and
b. providing a second plant comprising a vector according to any one of embodiments 50 to 60 or a set of at least two recombinant cassettes according to any one of embodiments 61 to 71; or
c. providing a first plant comprising a nucleic acid encoding the RNA guided effector proteins as described in any one of embodiment 7 to 18; and
d. providing a second plant comprising the target site and comprising a vector according to any one of embodiments 50 to 60 or a set of at least two recombinant cassettes according to any one of embodiments 61 to 71;
e. crossing the first and second plant and identifying a progeny plant having an edit at or near the target site the edit comprising insertion of at least one nucleotide, deletion of one nucleotide or substitution of at least one nucleotide, in at least a cell of the plant, optionally in essentially all cells of the plant.

In another aspect, the current invention provides methods and means for iterative modular cloning as expressed in the following embodiments.

Embodiment 92: A method for iterative modular cloning comprising the steps of
a. providing a first DNA fragment comprising a recombinant cassette of interest flanked by two different restriction sites, each site recognized respectively by a different rare cutting endonuclease, whereby the rare cutting endonucleases recognize a restriction site comprising at least 8 nucleotides and whereby the rare cutting endonucleases generate compatible cohesive ends;
b. treating the first DNA fragment with both rare cutting endonucleases of step a;
c. providing a DNA destination vector comprising one restriction site recognized by only one of the rare cutting endonucleases of step a;
d. treating the destination vector with the one rare cutting endonuclease of step c and optionally dephosphorylating the treated destination vector;
e. mixing the first DNA fragment resulting from step b with the destination vector of step d;
f. optionally adding a DNA ligase;
g. obtaining a resulting destination vector wherein the DNA fragment is linked to the destination vector whereby the recognition site recognized by only one of the rare cutting endonucleases of step c is reconstituted in the resulting destination vector;
h. providing a second DNA fragment comprising a recombinant cassette of interest flanked by two different restriction sites, each site recognized respectively by a different rare cutting endonuclease of step a;
i. treating the second DNA fragment with both rare cutting endonucleases of step a;
j. treating the resulting destination vector of step g with the one rare cutting endonuclease of step c and optionally dephosphorylating the treated destination vector;
k. mixing the second DNA fragment resulting from step i with the resulting destination vector of step j;
l. optionally adding a DNA ligase;
m. obtaining a second resulting destination vector wherein the second DNA fragment is linked to the resulting destination vector, whereby the recognition site recognized by only one of the rare cutting endonucleases of step c is reconstituted in the second resulting destination vector;
n. optionally reiterating steps a to g with a further DNA fragment comprising a recombinant cassette of interest flanked by the two different restriction sites to obtain a further resulting destination vector.

Embodiment 93: The method according to embodiment 92 wherein one of the two different rare cutting endonuclease is PacI and the other rare cutting endonuclease is AsiSI.

Embodiment 94: The method according to any one of embodiments 92 or 93 wherein the recognition site in the destination vector is PacI.

Embodiment 95: The method according to any one of embodiments 92 or 93 wherein the recognition site in the destination vector is AsiSI.

Embodiment 96: The method according to embodiment 92 wherein one of the two different rare cutting endonuclease is AscI and the other rare cutting endonuclease is MauB1.

Embodiment 97: The method according to any one of embodiments 92 or 96, wherein the recognition site in the destination vector is AscI.

Embodiment 98: The method according to any one of embodiments 92 or 96, wherein the recognition site in the destination vector is MauB1.

Embodiment 99: The method according to any one of embodiments 92 to 98 wherein the recombinant cassette of the first and/or second DNA fragment and/or further DNA fragment encodes one or more guide RNAs for targeted gene modification using Cas type nucleases.

Embodiment 100: A resulting destination vector or second resulting destination vector or further resulting destination vector produced by the method of any one of embodiments 92 to 99.

Embodiment 101: A cell comprising a resulting destination vector according to embodiment 100.

Embodiment 102: The cell according to embodiment 101, wherein the cell is a prokaryotic cell.

Embodiment 103: The cell according to embodiment 101, wherein the cell is a eukaryotic cell.

Embodiment 104: The cell according to embodiment 101, wherein the cell is a plant cell.

### BRIEF DESCRIPTION OF THE SEQUENCES IN THE SEQUENCE LISTING

SEQ ID NO 1: nucleotide sequence of an Oryza sativa Actin promoter SEQ ID NO 2: plant codon optimized nucleotide sequence encoding LbCas12a

Seq ID NO 3: nucleotide sequence of an Oryza sativa terminator for Cas12a expression cassette used in the Examples.

SEQ ID NO 4: Lachnospiraceae bacterium Cas12a protein amino acid sequence

SEQ ID NO 5: Franscisella novicida Cas12a protein amino acid sequence

SEQ ID NO 6: Acidaminococcus sp. Cas12a protein amino acid sequence

SEQ ID NO 7: nucleotide sequence of a spacer targeting GA20 oxidase

SEQ ID NO 8: nucleotide sequence of a spacer targeting Brown midrib 3

SEQ ID NO 9: nucleotide sequence of a direct repeat included in the guide RNA of the examples.

SEQ ID NO 10: nucleotide sequence of GSP2262, a synthetic Pol III promoter

SEQ ID NO 11: nucleotide sequence of GSP2273, a synthetic Pol III promoter

SEQ ID NO 12: nucleotide sequence of GSP2239, a synthetic Pol III promoter

SEQ ID NO 13: nucleotide sequence of GSP2244, synthetic Pol III promoter

SEQ ID NO 14: nucleotide sequence of ZmUbqM promoter

SEQ ID NO 15: nucleotide sequence of GSP2233 synthetic Pol III promoter

SEQ ID NO 16: nucleotide sequence of GSP2245 synthetic Pol III promoter

SEQ ID NO 17: nucleotide sequence of chimeric promoter chromosome 1-8

SEQ ID NO 18: nucleotide sequence of P-GSP2231 synthetic Pol III promoter

SEQ ID NO 19: nucleotide sequence of P-GSP2235 synthetic Pol III promoter

SEQ ID NO 20: nucleotide sequence of P-GSP2240 synthetic Pol III promoter

SEQ ID NO 21: nucleotide sequence of P-GSP2246 synthetic Pol III promoter

SEQ ID NO 22: nucleotide sequence of P-GSP2252 synthetic Pol III promoter

SEQ ID NO 23: nucleotide sequence of P-GSP2257 synthetic Pol III promoter

SEQ ID NO 24: nucleotide sequence of P-GSP2230 synthetic Pol III promoter

SEQ ID NO 25: nucleotide sequence of P-GSP2232 synthetic Pol III promoter

SEQ ID NO 26: nucleotide sequence of P-GSP2234 synthetic Pol III promoter

SEQ ID NO 27: nucleotide sequence of P-GSP2236 synthetic Pol III promoter

SEQ ID NO 28: nucleotide sequence of P-GSP2237 synthetic Pol III promoter

SEQ ID NO 29: nucleotide sequence of P-GSP2238 synthetic Pol III promoter

SEQ ID NO 30: nucleotide sequence of P-Zm.GSP2241 synthetic Pol III promoter

SEQ ID NO 31: nucleotide sequence of: P-Zm.GSP2242 synthetic Pol III promoter

SEQ ID NO 32: nucleotide sequence of P-Zm.GSP2243 synthetic Pol III promoter

SEQ ID NO 33: nucleotide sequence of: P-Zm.GSP2247 synthetic Pol III promoter

SEQ ID NO 34: nucleotide sequence of P-Zm.GSP2248 synthetic Pol III promoter

SEQ ID NO 35: nucleotide sequence of P-Zm.GSP2249 synthetic Pol III promoter

SEQ ID NO 36: nucleotide sequence of P-Zm.GSP2250 synthetic Pol III promoter

SEQ ID NO 37: nucleotide sequence of P-Zm.GSP2251 synthetic Pol III promoter

SEQ ID NO 38: nucleotide sequence of P-Zm.GSP2253 synthetic Pol III promoter

SEQ ID NO 39: nucleotide sequence of P-Zm.GSP2254 synthetic Pol III promoter

SEQ ID NO 40: nucleotide sequence of P-Zm.GSP2255 synthetic Pol III promoter

SEQ ID NO 41: nucleotide sequence of P-Zm.GSP2256 synthetic Pol III promoter

SEQ ID NO 42: nucleotide sequence of P-Zm.GSP2258 synthetic Pol III promoter

SEQ ID NO 43: nucleotide sequence of P-Zm.GSP2259 synthetic Pol III promoter

SEQ ID NO 44: nucleotide sequence of P-Zm.GSP2260 synthetic Pol III promoter

SEQ ID NO 45: nucleotide sequence of P-Zm.GSP2261 synthetic Pol III promoter

SEQ ID NO 46: nucleotide sequence of the Zea mays Ga20 oxidase_3 (Ga20ox_3) genomic region targeted for genome editing

SEQ ID NO 47: nucleotide sequence of the Zea mays Brown midrib 3 (Bmr3) genomic region targeted for genome editing Bmr3 targeted genomic sequence

SEQ ID NO 48: nucleotide sequence of the 5′ Nuclear Localization signal (NLS) for LbCas12a

SEQ ID NO 49: nucleotide sequence of the 5′ Nuclear Localization signal (NLS) for LbCas12a.

### BRIEF DESCRIPTION OF THE DRAWINGS

FIG. 1: Schematic representation of various gRNA expressing constructs described herein. Pol III promoters are indicated by arrows and have the prefix GSP. Direct repeats between the various spacers or guide RNAs are indicated by gray rectangles. Spacers or guide RNAs directed towards GA20ox_3 target genomic sequences are indicated by open diamonds. Spacers or guide RNAs directed towards Bmr_3 target genomic sequences are indicated by black diamonds.

FIG. 2: Editing rates obtained with variant constructs expressing guide RNAs targeting GA20_ox3 and Bmr3 genomic sequences using multiple single guide RNA or two guide RNA cassettes expressed under control of different Pol III promoters or using multiple guide RNA cassettes expressed under control of different Pol III promoters as described in Table 3 and Example 1.

FIG. 3: Editing rates obtained with various constructs expressing a guide RNA targeting Bmr3 genomic sequence under control of different Pol III promoters in protoplasts, as described in Table 4 and Example 2.

FIG. 4A and FIG. 4B: Advanceable editing rates obtained with various constructs expressing a guide RNA targeting Bmr3 genomic sequence under control of different Pol III promoters in planta, as described in Table 4 and Example 3.

FIG. 5. Schematic overview of the iterative modular cloning strategy described in Example 4 for guide RNA cassettes. Direct repeats between the various spacers or guide RNAs are indicated by gray rectangles. Spacers or guide RNAs are indicated by diamonds.

FIG. 6. Comparison of expression of mature crRNA targeting Zm.Bmr3, LbCas12a mRNA, or CP4 selectable marker across constructs. The number of cassettes and spacers in each construct are indicated. Significantly higher mean expression is marked with * or ** (Welch's ANOVA followed by Welch's t-test, p<0.05 or 0.01, respectively)

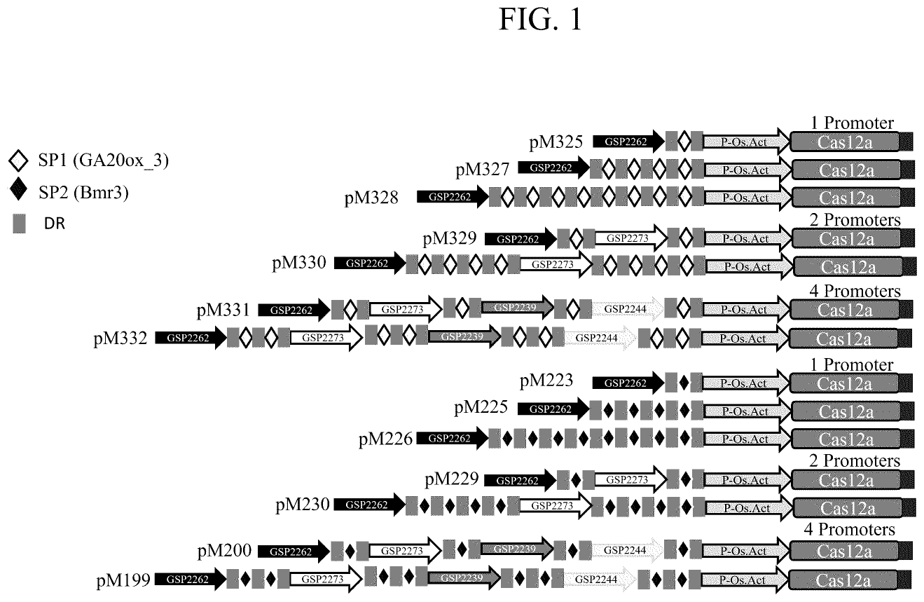

### DETAILED DESCRIPTION OF THE INVENTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of this disclosure includes, unless otherwise indicated, conventional techniques of biochemistry, chemistry, molecular biology, microbiology, cell biology, plant biology, genomics, biotechnology, and genetics, which are within the skill of the art. See, for example, Green and Sambrook, Molecular Cloning: A Laboratory Manual, 4th edition (2012); Current Protocols In Molecular Biology (F. M. Ausubel, et al. eds., (1987)); Plant Breeding Methodology (N.F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

As used herein, terms in the singular and the singular forms “a,” “an,” and “the,” for example, include plural referents unless the content clearly dictates otherwise.

Any composition, nucleic acid molecule, polypeptide, cell, plant, etc. provided herein is specifically envisioned for use with any method provided herein.

The current invention relates to methods for targeted gene modification in cells, using an RNA guided effector protein and at least two recombinant cassettes expressing the same or similar guide RNA under control of different promoters, as well as means for such methods, all as described in the above mentioned embodiments and elsewhere in this document.

Nucleic acids and amino acids

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise deoxyribonucleotides, ribonucleotides, or combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded.

As used herein, the term “recombinant construct” in reference to a nucleic acid (DNA or RNA) molecule, protein, construct, vector, etc., refers to a nucleic acid or amino acid molecule or sequence that is man-made and not normally found in nature, and/or is present in a context in which it is not normally found in nature, including a nucleic acid molecule (DNA or RNA) molecule, protein, construct, etc., comprising a combination of polynucleotide or protein sequences that would not naturally occur contiguously or in close proximity together without human intervention, and/or a polynucleotide molecule, protein, construct, etc., comprising at least two polynucleotide or protein sequences that are heterologous with respect to each other.

As used herein, the term “recombinant cassette” refers to a nucleic acid comprising a promoter sequence, a sequence of interest, in this context usually a guide RNA or an array of guide RNA sequences, and optionally a terminator sequence. The term “recombinant construct” may encompass a “recombinant cassette”. A recombinant construct may also be used interchangeable with a recombinant cassette.

As used herein, the term “heterologous” refers to a nucleotide/polypeptide that originates from a foreign species, or, if from the same species, is substantially modified from its native form in composition and/or genomic locus by deliberate human intervention. A “heterologous” or a “recombinant” nucleotide sequence is a nucleotide sequence not naturally associated with a host cell into which it is introduced, including non-naturally occurring multiple copies of a naturally occurring nucleotide sequence

In one aspect, methods and compositions provided herein comprise a vector. As used herein, the term “vector” refers to a DNA molecule used as a vehicle to carry exogenous genetic material into a cell.

In an aspect, one or more polynucleotide sequences from a vector are stably integrated into a genome of a plant. In an aspect, one or more polynucleotide sequences from a vector are stably integrated into a genome of a plant cell.

In an aspect, a first nucleic acid sequence and a second nucleic acid sequence are provided in a single vector. In another aspect, a first nucleic acid sequence is provided in a first vector, and a second nucleic acid sequence is provided in a second vector.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Without being limiting, nucleic acids can be detected using hybridization. Hybridization between nucleic acids is discussed in detail in Sambrook et. al. (1989, Molecular Cloning: A Laboratory Manual, 2nd Ed., Cold Spring Harbor Laboratory Press, Cold Spring Harbor, NY).

Polypeptides can be detected using antibodies. Techniques for detecting polypeptides using antibodies include enzyme linked immunosorbent t assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

The terms “percent sequence complementarity” or “percent complementarity” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

For optimal alignment of sequences to calculate their percent identity, various pair-wise or multiple sequence alignment algorithms and programs are known in the art, such as ClustalW or Basic Local Alignment Search Tool (BLAST®), etc., that can be used to compare the sequence identity or similarity between two or more nucleotide or protein sequences. Although other alignment and comparison methods are known in the art, the alignment and percent identity between two sequences (including the percent identity ranges described above) can be as determined by the ClustalW algorithm, see, e.g., Chenna R. et. al., “Multiple sequence alignment with the Clustal series of programs,” Nucleic Acids Research 31:3497-3500 (2003); Thompson JD ct. al., “Clustal W: Improving the sensitivity of progressive multiple sequence alignment through sequence weighting, position-specific gap penalties and weight matrix choice,” Nucleic Acids Research 22:4673-4680 (1994); Larkin MA ct. al., “Clustal W and Clustal X version 2.0,” Bioinformatics 23:2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

As used herein, a first nucleic acid molecule can “hybridize” a second nucleic acid molecule via non-covalent interactions (e.g., Watson-Crick base-pairing) in a sequence-specific, antiparallel manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymine (T), adenine (A) pairing with uracil (U), and guanine (G) pairing with cytosine (C). In addition, it is also known in the art that for hybridization between two RNA molecules (e.g., dsRNA), guaninc base pairs with uracil. For example, G/U base-pairing is partially responsible for the degeneracy (i.e., redundancy) of the genetic code in the context of tRNA anti-codon base-pairing with codons in mRNA. In the context of this disclosure, a guanine of a protein-binding segment (dsRNA duplex) of a subject DNA-targeting RNA molecule is considered complementary to an uracil, and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer nucleotides) the position of mismatches becomes important (see Sambrook et. al.). Typically, the length for a hybridizable nucleic acid is at least 10 nucleotides. Illustrative minimum lengths for a hybridizable nucleic acid are: at least 15 nucleotides; at least 18 nucleotides; at least 20 nucleotides; at least 22 nucleotides; at least 25 nucleotides; and at least 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST® programs (basic local alignment search tools) and PowerBLAST programs known in the art (see Altschul et. al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Promoters

In some aspects, the differing promoters of the at least two recombinant constructs or cassettes expressing the guide RNAs comprising the spacer sequence complementary to a target site, such as a genomic target site, as described herein, may be selected from plant-expressible promoters. In some aspects, the RNA guided effector protein may be expressed from a nucleic acid encoding said RNA guided effector operably linked to a promoter. In some aspects, the promoter may be a plant-expressible promoter

As used herein, a “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. A “promoter” may refer to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, a promoter functional in a plant may be used with the constructs of this invention. Non-limiting examples of a promoter useful for driving expression in a plant include the promoter of the RubisCo small subunit gene 1 (PrbcS1), the promoter of the actin gene (Pactin), the promoter of the nitrate reductase gene (Pnr) and the promoter of duplicated carbonic anhydrase gene 1 (Pdcal) (See, Walker et al. (2005) Plant Cell Rep. 23:727-735; Li et al. (2007) Gene 403:132-142; Li et al. (2010) Mol Biol. Rep. 37:1143-1154). PrbcS1 and Pactin are constitutive promoters and Pnr and Pdcal are inducible promoters. Pnr is induced by nitrate and repressed by ammonium (Li et al. (2007) Gene 403:132-142) and Pdcal is induced by salt (Li ct al. (2010) Mol Biol. Rep. 37:1143-1154). In some embodiments, a promoter useful with this invention is RNA polymerase II (Pol II) promoter.

Examples of constitutive promoters useful for plants include, but are not limited to, cestrum virus promoter (cmp) (U.S. Pat. No. 7,166,770), the rice actin 1 promoter (Wang et al. (1992) Mol. Cell. Biol. 12:3399-3406; as well as U.S. Pat. No. 5,641,876), CaMV 35S promoter (Odell et al. (1985) Nature 313:810-812), CaMV 19S promoter (Lawton et al. (1987) Plant Mol. Biol. 9:315-324), nos promoter (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and the ubiquitin promoter. The constitutive promoter derived from ubiquitin accumulates in many cell types. Ubiquitin promoters have been cloned from several plant species for use in transgenic plants, for example, sunflower (Binet et al. (1991) Plant Science 79:87-94), maize (Christensen et al. (1989) Plant Molcc. Biol. 12:619-632), and Arabidopsis (Norris et al. (1993) Plant Molec. Biol. 21:895-906). The maize ubiquitin promoter (UbiP) has been developed in transgenic monocot systems and its sequence and vectors constructed for monocot transformation are disclosed in the patent publication EP 0 342 926. The ubiquitin promoter is suitable for the expression of the nucleotide sequences of the invention in transgenic plants, especially monocotyledons. Further, the promoter expression cassettes described by McElroy et al. ((1991) Mol. Gen. Genet. 231:150-160) can be casily modified for the expression of the nucleotide sequences of the invention and are particularly suitable for use in monocotyledonous hosts.

In some embodiments, tissue specific/tissue preferred promoters can be used for expression of a heterologous polynucleotide in a plant cell. Tissue specific or preferred expression patterns include, but are not limited to, green tissue specific or preferred, root specific or preferred, stem specific or preferred, flower specific or preferred or pollen specific or preferred. Promoters suitable for expression in green tissue include many that regulate genes involved in photosynthesis and many of these have been cloned from both monocotyledons and dicotyledons. In one embodiment, a promoter useful with the invention is the maize PEPC promoter from the phosphoenol carboxylase gene (Hudspeth & Grula (1989) Plant Molec. Biol. 12:579-589). Non-limiting examples of tissue-specific promoters include those associated with genes encoding the seed storage proteins (such as B-conglycinin, cruciferin, napin and phascolin), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP U.S. Pat. No. 255,378). Tissue-specific or tissue-preferential promoters useful for the expression of the nucleotide sequences of the invention in plants, particularly maize, include but are not limited to those that direct expression in root, pith, leaf, or pollen. Such promoters are disclosed, for example, in WO 93/07278, herein incorporated by reference in its entirety. Other non-limiting examples of tissue specific or tissue preferred promoters useful with the invention the cotton rubisco promoter disclosed in U.S. Pat. No. 6,040,504; the rice sucrose synthase promoter disclosed in U.S. Pat. No. 5,604,121; the root specific promoter described by de Framond ((1991) FEBS 290:103-106; EP 0 452 269 to Ciba-Geigy); the stem specific promoter described in U.S. Pat. No. 5,625,136 (to Ciba-Geigy) and which drives expression of the maize trpA gene; the cestrum yellow leaf curling virus promoter disclosed in WO 01/73087; and pollen specific or preferred promoters including, but not limited to, ProOsLPS10 and ProOsLPS11 from rice (Nguyen et al. (2015) Plant Biotechnol. Reports 9 (5): 297-306), ZmSTK2_USP from maize (Wang et al. (2017) Genome 60 (6): 485-495), LAT52 and LAT59 from tomato (Twell et al. (1990) Development 109 (3): 705-713), Zm13 (U.S. Pat. No. 10,421,972), PLA2-8 promoter from Arabidopsis (U.S. Pat. No. 7,141,424), and/or the ZmC5 promoter from maize (International PCT Publication No. WO1999/042587.

Additional examples of plant tissue-specific/tissue preferred promoters include, but are not limited to, the root hair-specific cis-elements (RHEs) (Kim et al. (2006) The Plant Cell 18:2958-2970), the root-specific promoters RCc3 (Jeong et al. (2010) Plant Physiol. 153:185-197) and RB7 (U.S. Pat. No. 5,459,252), the lectin promoter (Lindstrom et al. (1990) Der. Genct. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology 37 (8): 1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-1,5-bisphosphate carboxylase” pp. 29-39 In: Genetic Engineering of Plants (Hollaender ed., Plenum Press 1983; and Poulsen et al. (1986) Mol. Gen. Genct. 205:193-200), Ti plasmid mannopine synthase promoter (Langridge et al. (1989) Proc. Natl. Acad. Sci. USA 86:3219-3223), Ti plasmid nopaline synthase promoter (Langridge et al. (1989) supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), a-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBO J. 10:2605-2612).

Useful for seed-specific expression is the pea vicilin promoter (Czako et al. (1992) Mol. Gen. Genct. 235:33-40; as well as the seed-specific promoters disclosed in U.S. Pat. No. 5,625,136. Useful promoters for expression in mature leaves are those that are switched at the onset of senescence, such as the SAG promoter from Arabidopsis (Gan et al. (1995) Science 270:1986-1988).

Plant-expressible promoters useful for the methods and compositions herein described also include egg cell-preferred or embryo-tissue preferred promoters as described in WO2022/056139 (incorporated herein in its entirety), such as a DSULI promoter, an EAl promoter, an ES4 promoter, a DMC1 promoter, a Mps1 promoter, an Adf1 promoter or an EAL promoter.

Other plant-expressible promoters useful for the invention include floral-tissue preferred or floral cell-preferred promoter as described in PCT/US2023/065042 (incorporated herein in its entirety).

In addition, promoters functional in chloroplasts can be used. Non-limiting examples of such promoters include the bacteriophage T3 gene 9 5′ UTR and other promoters disclosed in U.S. Pat. No. 7,579,516. Other promoters useful with the invention include but are not limited to the S-E9 small subunit RuBP carboxylase promoter and the Kunitz trypsin inhibitor gene promoter (Kti3).

In some embodiments, the differing promoters of the at least two recombinant constructs or cassettes expressing the guide RNAs comprising the spacer sequence complementary to a target site, such as a genomic target site, as described herein, may be selected from RNA polymerase III (Pol III) promoters. In some aspects, the Pol III promoter may be a U6 promoter, an H1 promoter, a 5S promoter, an Adenovirus 2 (Ad2) VAI promoter, a tRNA promoter, and a 7SK promoter. Sec, for example, Schramm and Hernandez, 2002, Genes & Development, 16:2593-2620, which is incorporated by reference herein in its entirety.

In some aspects, the Pol III promoters may be derived from small nuclear RNA (snRNA) encoding genes. In some aspects, the Pol III promoters may be selected from the corn, tomato and soybean U6, U3, U2, U5 and 7SL snRNA promoters disclosed in WO2015/131101 (incorporated herein by reference in its entirety) including the snRNA promoter sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20; SEQ ID NOs: 146-149, SEQ ID NOs: 160-166, SEQ ID NOs: 201 or SEQ ID NO: 283, included therein in the accompanying sequence listing.

In some aspects, the Pol III promoters may be synthetic snRNA promoters, such as the snRNA promoters described in WO2022/232407 (incorporated herein by reference in its entirety) including the snRNA promoter sequences of SEQ ID Nos: 1-10 included therein in the accompanying sequence listing.

In some aspects, the Pol III promoters may be selected from Pol III promoters comprising a nucleotide sequence of any one of SEQ ID NOs: 10-13, 15-16 or 18-45 of the accompanying

### SEQUENCE LISTING

In some aspects, the Pol III promoters may be chimeric Pol III promoters. In some aspects, the Pol III promoters may be variants of the Pol III promoters disclosed herein. In some aspects, a variant of a Pol III promoters comprising a sequence that, when optimally aligned to the reference sequence has at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, or at least about 99 percent identity to the reference sequence and having promoter activity as disclosed herein are provided. Variants of the Pol III promoters may comprise a nucleotide sequence having at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, or at least about 99 percent identity of a nucleotide sequence of any one of SEQ ID NOs: 10-13, 15-16 or 18-45.

In some aspects, fragments of the Pol III promoters disclosed herein may be used according to the invention, wherein the fragments comprise at least about 50, at least about 75, at least about 95, at least about 100, at least about 125, at least about 150, at least about 175, at least about 200, at least about 225, at least about 250, at least about 275, at least about 300, at least about 325, at least about 350, at least about 375, at least about 400 contiguous nucleotides, at least about 425, at least about 450, at least about 475, or longer, of a DNA molecule having promoter activity as disclosed herein. In certain embodiments, provided are fragments of a small nuclear RNA promoter provided herein, having gene expression activity. Fragments of a Pol III promoter may comprise at least about 50, at least about 75, at least about 95, at least about 100, at least about 125, at least about 150, at least about 175, at least about 200, at least about 225, at least about 250, at least about 275, at least about 300, at least about 325, at least about 350, at least about 375, at least about 400, at least about 425, at least about 450, at least about 475, or longer contiguous nucleotides of any one of SEQ ID Nos: 10-13, 15-16 or 18-45.

### Regulatory Elements

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression. As an example, a promoter/intron combination useful with this invention includes but is not limited to that of the maize Ubil promoter and intron.

Non-limiting examples of introns useful with the present invention include introns from the ADHI gene (e.g., Adh1-S introns 1, 2 and 6), the ubiquitin gene (Ubil), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdcal), the psbA gene, the atpA gene, or any combination thereof.

### Guide Nucleic Acids

As used herein, a “guide nucleic acid” refers to a nucleic acid that forms a ribonucleoprotein (e.g., a complex) with a guided nuclease (e.g., without being limiting, Cas12a, CasX) and then guides the ribonucleoprotein to a specific sequence in a target nucleic acid molecule, where the guide nucleic acid and the target nucleic acid molecule share complementary sequences. In an aspect, a ribonucleoprotein provided herein comprises at least one guide nucleic acid.

In an aspect, a guide nucleic acid comprises DNA. In another aspect, a guide nucleic acid comprises RNA. In an aspect, a guide nucleic acid comprises DNA, RNA, or a combination thereof. In an aspect, a guide nucleic acid is single-stranded. In another aspect, a guide nucleic acid is at least partially double-stranded.

When a guide nucleic acid comprises RNA, it can be referred to as a “guide RNA.” In another aspect, a guide nucleic acid comprises DNA and RNA. In another aspect, a guide RNA is single-stranded. In another aspect, a guide RNA is double-stranded. In a further aspect, a guide RNA is partially double-stranded.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12c, Cas13a, Cas13b, Cas13c, Cas13d, Cas1, CaslB, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Csc2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer. A guide RNA may comprise a donor template for introducing specific modifications in the target sequence.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. (2007) Nucleic Acids Res. 35 (Web Server issue): W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to portion of a target nucleic acid (e.g., target DNA) (e.g., protospacer) . . . . A spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. In some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length. In some embodiments, a spacer sequence may comprise any one of the sequences of SEQ ID NOs: 7-8, or any combination thereof.

In some embodiments, the 5′ region of a spacer sequence of a guide nucleic acid may be identical to a target DNA, while the 3′ region of the spacer may be substantially complementary to the target DNA (such as a spacer of a Type V CRISPR-Cas system), or the 3′ region of a spacer sequence of a guide nucleic acid may be identical to a target DNA, while the 5′ region of the spacer may be substantially complementary to the target DNA (such as a spacer of a Type II CRISPR-Cas system), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

In an aspect, a guide nucleic acid comprises a guide RNA. In another aspect, a guide nucleic acid comprises at least one guide RNA. In another aspect, a guide nucleic acid comprises at least two guide RNAs. In another aspect, a guide nucleic acid comprises at least three guide RNAs. In another aspect, a guide nucleic acid comprises at least five guide RNAs. In another aspect, a guide nucleic acid comprises at least ten guide RNAs.

In another aspect, a guide nucleic acid comprises at least 10 nucleotides. In another aspect, a guide nucleic acid comprises at least 11 nucleotides. In another aspect, a guide nucleic acid comprises at least 12 nucleotides. In another aspect, a guide nucleic acid comprises at least 13 nucleotides. In another aspect, a guide nucleic acid comprises at least 14 nucleotides. In another aspect, a guide nucleic acid comprises at least 15 nucleotides. In another aspect, a guide nucleic acid comprises at least 16 nucleotides. In another aspect, a guide nucleic acid comprises at least 17 nucleotides. In another aspect, a guide nucleic acid comprises at least 18 nucleotides. In another aspect, a guide nucleic acid comprises at least 19 nucleotides. In another aspect, a guide nucleic acid comprises at least 20 nucleotides. In another aspect, a guide nucleic acid comprises at least 21 nucleotides. In another aspect, a guide nucleic acid comprises at least 22 nucleotides. In another aspect, a guide nucleic acid comprises at least 23 nucleotides. In another aspect, a guide nucleic acid comprises at least 24 nucleotides. In another aspect, a guide nucleic acid comprises at least 25 nucleotides. In another aspect, a guide nucleic acid comprises at least 26 nucleotides. In another aspect, a guide nucleic acid comprises at least 27 nucleotides. In another aspect, a guide nucleic acid comprises at least 28 nucleotides. In another aspect, a guide nucleic acid comprises at least 30 nucleotides. In another aspect, a guide nucleic acid comprises at least 35 nucleotides. In another aspect, a guide nucleic acid comprises at least 40 nucleotides. In another aspect, a guide nucleic acid comprises at least 45 nucleotides. In another aspect, a guide nucleic acid comprises at least 50 nucleotides.

In another aspect, a guide nucleic acid comprises between 10 nucleotides and 50 nucleotides. In another aspect, a guide nucleic acid comprises between 10 nucleotides and 40 nucleotides. In another aspect, a guide nucleic acid comprises between 10 nucleotides and 30 nucleotides. In another aspect, a guide nucleic acid comprises between 10 nucleotides and 20 nucleotides. In another aspect, a guide nucleic acid comprises between 16 nucleotides and 28 nucleotides. In another aspect, a guide nucleic acid comprises between 16 nucleotides and 25 nucleotides. In another aspect, a guide nucleic acid comprises between 16 nucleotides and 20 nucleotides.

In an aspect, a guide nucleic acid comprises at least 70% sequence complementarity to a target site. In an aspect, a guide nucleic acid comprises at least 75% sequence complementarity to a target site. In an aspect, a guide nucleic acid comprises at least 80% sequence complementarity to a target site. In an aspect, a guide nucleic acid comprises at least 85% sequence complementarity to a target site. In an aspect, a guide nucleic acid comprises at least 90% sequence complementarity to a target site. In an aspect, a guide nucleic acid comprises at least 91% sequence complementarity to a target site. In an aspect, a guide nucleic acid comprises at least 92% sequence complementarity to a target site. In an aspect, a guide nucleic acid comprises at least 93% sequence complementarity to a target site. In an aspect, a guide nucleic acid comprises at least 94% sequence complementarity to a target site. In an aspect, a guide nucleic acid comprises at least 95% sequence complementarity to a target site. In an aspect, a guide nucleic acid comprises at least 96% sequence complementarity to a target site. In an aspect, a guide nucleic acid comprises at least 97% sequence complementarity to a target site. In an aspect, a guide nucleic acid comprises at least 98% sequence complementarity to a target site. In an aspect, a guide nucleic acid comprises at least 99% sequence complementarity to a target site. In an aspect, a guide nucleic acid comprises 100% sequence complementarity to a target site. In another aspect, a guide nucleic acid comprises between 70% and 100% sequence complementarity to a target site. In another aspect, a guide nucleic acid comprises between 80% and 100% sequence complementarity to a target site. In another aspect, a guide nucleic acid comprises between 90% and 100% sequence complementarity to a target site.

In an aspect, a guide nucleic acid is capable of hybridizing to a target site.

As noted above, some guided nucleases, such as CasX and Cas9, require another non-coding RNA component, referred to as a trans-activating crRNA (tracrRNA), to have functional activity. Guide nucleic acid molecules provided herein can combine a crRNA and a tracrRNA into one nucleic acid molecule in what is herein referred to as a “single guide RNA” (sgRNA). The gRNA guides the active CasX complex to a target site within a target sequence, where CasX can cleave the target site. In other embodiments, the crRNA and tracrRNA are provided as separate nucleic acid molecules.

In an aspect, a guide nucleic acid comprises a crRNA. In another aspect, a guide nucleic acid comprises a tracrRNA. In a further aspect, a guide nucleic acid comprises a sgRNA.

It is expected that the methods and compositions disclosed herein are useful to increase the editing efficiency of a guide RNA which results in moderate or poor editing or has a moderate or low editing efficiency at the target site when expressed as a single guide RNA, or from a single recombinant construct, in combination with an RNA guided effector protein. As used herein “a guide RNA with low editing efficiency” results in a cutting efficiency of less than 30%, less than 25% or less than 20% when assayed, e.g. in an assay involving introduction of such guide RNA or a nucleic acid construct encoding such guide RNA in plant protoplast comprising a target site recognized by the guide RNA and determination of the percentage of protoplasts comprising insertions/deletions at the target site e.g. by determination of the number of reads by sequencing. The potential editing efficiency of a particular guide RNA can be estimated in silico as described in US2023091138 (herein incorporated in its entirety).

Briefly, in this document a methods is provided which includes, for each of multiple guide nucleic acid sequences, for a desired edit: identifying characteristics of the guide nucleic acid sequence and/or sequence segment; assigning, based on a scoring data structure, a score to the guide nucleic acid sequence for each identified characteristic; and aggregating the assigned scores into an edit score for the guide nucleic acid sequence. The method then includes compiling a report that includes the multiple guide nucleic acid sequences and the edit score for each of the guide nucleic acid sequences, thereby permitting selection, from the report, of at least one of the guide nucleic acid sequences based on the associated edit score. Additionally, based on the edit score, a number of guide nucleic acid sequences tested, a sample size, and/or a number of experiments can be set to reach the desired edit.

Scores in the scoring data structure for individual characteristics may be determined by leveraging experimental data indicative of the effectiveness of gRNAs based on one or more of the characteristics, as exemplified by the scoring data structure in the below Table. For example, gRNAs were tested to determine an effectivity rate. And, in connection therewith, the different, individual characteristics (alone and/or in combination) are identified as more often associated with relatively high or low efficacy gRNAs. Those characteristics associated with high efficacy gRNAs were set to positive values, whereas those characteristics associated with moderate or low efficacy gRNAs were set to negative values. In this manner, the scores included in the scoring data structure are trained based on determined effectivity rates, separately for the target organism (e.g., corn or soy), and then retrained as needed. Scores for one or more characteristics for other organisms could be trained, and retrained, in a similar manner. In the below table, the example scoring data structure includes different characteristics of a guide nucleic acid (e.g., of a gRNA sequence, etc.) and sequence segment, and also includes a score (for example, for corn and soy (as the given organism)) based on the presence or absence of the characteristic in the given gRNA (e.g., GC content, Ts, TTs, 5′ T, 5′ G, 5′ C, 5′A, etc.) and sequence segment (e.g., TTTC PAM, TTTG PAM, etc.). The below table also includes a category or range for characteristics associated with such a category or range, and a score associated with the range of the characteristic. Further in the below table, as shown, the scores are distinct for different plants, or more broadly, organisms (e.g., corn and soy etc.). For instance, for a gRNA sequence characteristic of GC content, where the category (or range) is less than 30 (>30) the score for corn is-1 and the score for soy is-1. For a gRNA sequence characteristic of GC content, where the category (or range) is between 50 and 60, the score for corn is 1 and the score for soy is 1. Or, for a gRNA sequence characteristic of G at position 6, the score for corn is-0.25 while the score for soy is 0. It should be appreciated that the example scoring data structure in the table below is merely an example, and that other scoring data structures (with other data) may be included in other system embodiments. Additionally, other tables including the same or different characteristics and/or associated scores may be included in other system embodiments.

| | |
|---|---|
| gRNA or Sequence | Score per plant |

| Segment characteristic | Category (or Range) | Corn | Soy |
|---|---|---|---|
| GC content | <30 | −1 | −1 |
| GC content | Between 30 and 40 | 0.25 | 0.25 |
| GC content | Between 40 and 50 | 0.5 | 0.5 |
| GC content | Between 50 and 60 | 1 | 1 |
| GC content | Between 60 and 70 | 1.25 | 1.25 |
| GC content | >70 | −1 | −1 |
| Ts | >6 | −0.25 | −0.25 |
| TTTC PAM | | −0.5 | −0.5 |
| TTTG PAM | | 0 | 0.25 |
| TTs | >2 | −1 | −1 |
| TTs | 0 | 0.25 | 0.25 |
| 5′T | | −1 | −1 |
| 5′G | | 1 | 1 |
| 5′C | | −0.25 | −0.25 |
| 5′A | | −0.5 | −0.5 |
| A at position 6 | | 0.25 | 0 |
| G at position 6 | | −0.25 | 0 |
| C at position 6 | | −0.25 | −0.25 |
| C at 23 | | 0.25 | 0.25 |
| A at 23 | | −0.25 | −0.25 |
| G at 23 | | −0.25 | −0.25 |
| A6 + C23 | | 0.75 | 0.5 |
| #Gs 19-22 | >=3 | −0.5 | −0.5 |
| #Gs 19-22 | > | −0.25 | −0.25 |

The combined scores for different gRNAs can then be used to assess identified gRNAs relative to one another. The table below summarizes experimentally defined editing efficiencies for guide RNAs with an estimated score of <1 and >=1. There is an about 20% editing rate difference for guide RNAs with a score of >=1 versus a score of <1.

| | all | Corn | Soy |
|---|---|---|---|
| #gRNA tested | 516 | 141 | 375 |
| Average editing efficiency | 27.8% | 26.7% | 28.1% |
| for gRNA with | | | |
| score <1 | | | |
| average editing efficiency | 47.6% | 46.1% | 48.4% |
| for gRNA with score >=1 | | | |

The compositions and methods described herein are particularly suited to be applied using guide RNAs which result in moderate or poor editing or have a moderate or low editing efficiency at the target site defined by the guide RNA. In some aspects, the guide RNAs for which the methods and compositions of the invention are particularly beneficial have an editing efficiency score of less than 1, such as an editing efficiency score between 0 and 1 or less than 0.

### RNA Guided Nucleases

Guided nucleases are nucleases that form a complex (e.g., a ribonucleoprotein) with a guide nucleic acid molecule (e.g., a guide RNA), which then guides the complex to a target site within a target sequence. One non-limiting example of guided nucleases are CRISPR nucleases.

CRISPR (Clustered Regularly Interspaced Short Palindromic Repeats) nucleases (e.g., Cas9, CasX, Cas12a (also referred to as Cpf1), CasY, MAD7®) are proteins found in bacteria that are guided by guide RNAs (“gRNAs”) to a target nucleic acid molecule, where the endonuclease can then cleave one or two strands the target nucleic acid molecule. Although the origins of CRISPR nucleases are bacterial, many CRISPR nucleases have been shown to function in eukaryotic cells.

While not being limited by any particular scientific theory, a CRISPR nuclease forms a complex with a guide RNA (gRNA), which hybridizes with a complementary target site, thereby guiding the CRISPR nuclease to the target site. In class II CRISPR-Cas systems, CRISPR arrays, including spacers, are transcribed during encounters with recognized invasive DNA and are processed into small interfering CRISPR RNAs (crRNAs). The crRNA comprises a repeat sequence and a spacer sequence which is complementary to a specific protospacer sequence in an invading pathogen. The spacer sequence can be designed to be complementary to target sequences in a eukaryotic genome.

CRISPR nucleases associate with their respective crRNAs in their active forms. CasX, similar to the class II endonuclease Cas9, requires another non-coding RNA component, referred to as a trans-activating crRNA (tracrRNA), to have functional activity. Nucleic acid molecules provided herein can combine a crRNA and a tracrRNA into one nucleic acid molecule in what is herein referred to as a “single guide RNA” (sgRNA). Cas12a or MAD7® do not require a tracrRNA to be guided to a target site; a crRNA alone is sufficient for Cas12a or MAD7®. The gRNA guides the active CRISPR nuclease complex to a target site, where the CRISPR nuclease can cleave the target site.

When an RNA-guided CRISPR nuclease and a guide RNA form a complex, the whole system is called a “ribonucleoprotein.” Ribonucleoproteins provided herein can also comprise additional nucleic acids or proteins.

A prerequisite for cleavage of the target site by a CRISPR ribonucleoprotein is the presence of a conserved Protospacer Adjacent Motif (PAM) near the target site. Depending on the CRISPR nuclease, cleavage can occur within a certain number of nucleotides (e.g., between 18-23 nucleotides for Cas12a) from the PAM site. PAM sites are only required for type I and type II CRISPR associated proteins, and different CRISPR endonucleases recognize different PAM sites. Without being limiting, Cas12a can recognize at least the following PAM sites: TTTN, and YTN; CasX can recognize at least the following PAM sites: TTCN, TTCA, and TTC and MAD7® nuclease recognizes T-rich PAM sequences YTTN and seems to prefer TTTN to CTTN PAMs (where T is thymine; C is cytosine; A is adenine; Y is thymine or cytosine; and N is thymine, cytosine, guanine, or adenine).

Cas12a is an RNA-guided nuclease of a class II, type V CRISPR/Cas system. Cas12a nucleases generate staggered cuts when cleaving a double-stranded DNA molecule. Staggered cuts of double-stranded DNA produce a single-stranded DNA overhang of at least one nucleotide. This is in contrast to a blunt-end cut (such as those generated by Cas9), which does not produce a single-stranded DNA overhang when cutting double-stranded DNA.

In an aspect, a Cas12a nuclease provided herein is a Lachnospiraceae bacterium Cas12a (LbCas12a) nuclease. In another aspect, a Cas12a nuclease provided herein is a Francisella novicida Cas12a (FnCas12a) nuclease. In an aspect, a Cas12a nuclease is selected from the group consisting of LbCas12a and FnCas12a.

In an aspect, a Cas12a nuclease, or a nucleic acid encoding a Cas12a nuclease, is derived from a bacteria genus selected from the group consisting of Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, Acidaminococcus, Peregrinibacteria, Butyrivibrio, Parcubacteria, Smithella, Candidatus, Moraxella, and Leptospira.

In an aspect, a Cas12a nuclease is encoded by a polynucleotide comprising a sequence at least 80% identical to a polynucleotide of SEQ ID NO: 2. In another aspect, a Cas12a nuclease is encoded by a polynucleotide comprising a sequence at least 85% identical to a polynucleotide of SEQ ID NO: 2. In another aspect, a Cas12a nuclease is encoded by a polynucleotide comprising a sequence at least 90% identical to a polynucleotide of SEQ ID NO: 2. In another aspect, a Cas12a nuclease is encoded by a polynucleotide comprising a sequence at least 95% identical to a polynucleotide of SEQ ID NO: 2. In another aspect, a Cas12a nuclease is encoded by a polynucleotide comprising a sequence at least 96% identical to a polynucleotide selected from the group consisting of SEQ ID NO: 2. In another aspect, a Cas12a nuclease is encoded by a polynucleotide comprising a sequence at least 97% identical to a polynucleotide of SEQ ID NO: 2. In another aspect, a Cas12a nuclease is encoded by a polynucleotide comprising a sequence at least 98% identical to a polynucleotide of SEQ ID NO: 2. In another aspect, a Cas12a nuclease is encoded by a polynucleotide comprising a sequence at least 99% identical to a polynucleotide of SEQ ID NO: 2. In another aspect, a Cas12a nuclease is encoded by a polynucleotide comprising a sequence 100% identical to a polynucleotide of SEQ ID NO: 2.

In an aspect, a Cas12a nuclease provided herein comprises an amino acid sequence having at least 80% identical to an amino acid sequence selected from SEQ ID NO: 4, SEQ ID NO: 5 or SEQ ID NO: 6. In another aspect, a Cas12a nuclease provided herein comprises an amino acid sequence having at least 85% identical to an amino acid sequence selected from SEQ ID NO: 4, SEQ ID NO: 5 or SEQ ID NO: 6. In another aspect, a Cas12a nuclease provided herein comprises an amino acid sequence having at least 90% identical to an amino acid sequence selected from SEQ ID NO: 4, SEQ ID NO: 5 or SEQ ID NO: 6. In another aspect, a Cas12a nuclease provided herein comprises an amino acid sequence having at least 95% identical to an amino acid sequence selected from SEQ ID NO: 4, SEQ ID NO: 5 or SEQ ID NO: 6. In another aspect, a Cas12a nuclease provided herein comprises an amino acid sequence having at least 96% identical to an amino acid sequence selected from SEQ ID NO: 4, SEQ ID NO: 5 or SEQ ID NO: 6. In another aspect, a Cas12a nuclease provided herein comprises an amino acid sequence having at least 97% identical to an amino acid sequence selected from SEQ ID NO: 4, SEQ ID NO: 5 or SEQ ID NO: 6. In another aspect, a Cas12a nuclease provided herein comprises an amino acid sequence having at least 98% identical to an amino acid sequence selected from SEQ ID NO: 4, SEQ ID NO: 5 or SEQ ID NO: 6. In another aspect, a Cas12a nuclease provided herein comprises an amino acid sequence having at least 99% identical to an amino acid sequence selected from SEQ ID NO: 4, SEQ ID NO: 5 or SEQ ID NO: 6. In another aspect, a Cas12a nuclease provided herein comprises an amino acid sequence having at 100% identity to an amino acid sequence selected from SEQ ID NO: 4, SEQ ID NO: 5 or SEQ ID NO: 6.

In an aspect, a Cas12a provided herein is a variant Lachnospiraceae bacterium Cas12a (LbCas12a) nuclease with enhanced DNA cleavage activities at non-canonical TTTT protospacer adjacent motifs such as described in US2021/0348144 (incorporated herein by reference in its entirety) In another aspect, a Cas12a provided herein is a variant Lachnospiraceae bacterium Cas12a (LbCas12a) nuclease with enhanced activity as described in US20230040148 (incorporated herein by reference in its entirety) such as the LbCas12a-ultra having an N527R and E795L substitution in its amino acid sequence (reference amino acid sequence is SEQ ID NO: 4)

In an aspect, a Cas12a provided herein provided herein is a variant Lachnospiraceae bacterium Cas12a (LbCas12a) nuclease recognizing a PAM variant TYCV having a G532R and K595R substitution in its amino acid sequence (reference amino acid sequence is SEQ ID NO: 4) or a variant Lachnospiraceae bacterium Cas12a (LbCas12a) nuclease recognizing a PAM variant TATT having a G532R, K538R and Y524R substitution in its amino acid sequence (reference amino acid sequence is SEQ ID NO: 4) as disclosed in WO2016205711 (herein incorporated by reference in its entirety).

CasX is a type of class II CRISPR-Cas nuclease that has been identified in the bacterial phyla Deltaproteobacteria and Planctomycetes. Similar to Cas12a, CasX nucleases generate staggered cuts when cleaving a double-stranded DNA molecule. However, unlike Cas12a, CasX nucleases require a crRNA and a tracrRNA, or a single-guide RNA, in order to target and cleave a target nucleic acid.

In an aspect, a CasX nuclease provided herein is a CasX nuclease from the phylum Deltaproteobacteria. In another aspect, a CasX nuclease provided herein is a CasX nuclease from the phylum Planctomycetes. Without being limiting, additional suitable CasX nucleases are those set forth in WO 2019/084148, which is incorporated by reference herein in its entirety.

MAD7® (also known as ErCas12a) is an engineered nuclease of the Class 2 type V-A CRISPR-Cas (Cas12a/Cpf1) family with a low level of homology to canonical Cas12a nucleases. MAD7® nucleases generate staggered cuts when cleaving a double-stranded DNA molecule.MAD7® nuclease was initially identified in Eubacterium rectale. It only requires a crRNA like canonical Cas12a. An ErCas12a/MAD7® encoding nucleotide sequence can be found in the supplementary data (sequences S1) provided with Lin et al., 2021, Journal of Genetics and Genomics 48, pages 444-451)

In an aspect, a guided nuclease capable of generating a staggered cut in a double-stranded DNA molecule is selected from the group consisting of Cas12a; MAD7® and CasX. In an aspect, a guided nuclease is selected from the group consisting of Cas12a, MAD7® and CasX.

In an aspect, a guided nuclease is a RNA-guided nuclease. In another aspect, a guided nuclease is a CRISPR nuclease. In another aspect, a guided nuclease is a Cas12a nuclease. In another aspect, a guided nuclease is a CasX nuclease. In another aspect, a guided nuclease is a MAD7® nuclease.

As used herein, a “nuclear localization signal” (NLS) refers to an amino acid sequence that “tags” a protein for import into the nucleus of a cell. In an aspect, a nucleic acid molecule provided herein encodes a nuclear localization signal. In another aspect, a nucleic acid molecule provided herein encodes two or more nuclear localization signals.

In an aspect, a Cas12a nuclease provided herein comprises a nuclear localization signal. In an aspect, a nuclear localization signal is positioned on the N-terminal end of a Cas12a nuclease. In a further aspect, a nuclear localization signal is positioned on the C-terminal end of a Cas12a nuclease. In yet another aspect, a nuclear localization signal is positioned on both the N-terminal end and the C-terminal end of a Cas12a nuclease.

In an aspect, a CasX nuclease provided herein comprises a nuclear localization signal. In an aspect, a nuclear localization signal is positioned on the N-terminal end of a CasX nuclease. In a further aspect, a nuclear localization signal is positioned on the C-terminal end of a CasX nuclease. In yet another aspect, a nuclear localization signal is positioned on both the N-terminal end and the C-terminal end of a CasX nuclease.

In an aspect, a MAD7® nuclease provided herein comprises a nuclear localization signal. In an aspect, a nuclear localization signal is positioned on the N-terminal end of a MAD7® nuclease. In a further aspect, a nuclear localization signal is positioned on the C-terminal end of a MAD7® nuclease. In yet another aspect, a nuclear localization signal is positioned on both the N-terminal end and the C-terminal end of a MAD7® nuclease

In an aspect, a ribonucleoprotein comprises at least one nuclear localization signal. In another aspect, a ribonucleoprotein comprises at least two nuclear localization signals.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www [dot]kazusa [dot] or [dot]jp [forwards slash]codon and these tables can be adapted in a number of ways. Sce Nakamura et al., 2000, Nucl. Acids Res. 28:292. Computer algorithms for codon optimizing a particular sequence for expression in a particular plant cell are also available, such as Gene Forge (Aptagen; Jacobus, PA), are also available.

As used herein, “codon optimization” refers to a process of modifying a nucleic acid sequence for enhanced expression in a plant cell of interest by replacing at least one codon (e.g., at least 1, 2, 3, 4, 5, 10, 15, 20, 25, 50, or more codons) of a sequence with codons that are more frequently or most frequently used in the genes of the plant cell while maintaining the original amino acid sequence (e.g., introducing silent mutations).

In an aspect, one or more codons (e.g., 1, 2, 3, 4, 5, 10, 15, 20, 25, 50, or more, or all codons) in a sequence encoding a guided nuclease correspond to the most frequently used codon for a particular amino acid. In another aspect, one or more codons (e.g., 1, 2, 3, 4, 5, 10, 15, 20, 25, 50, or more, or all codons) in a sequence encoding a Cas12a nuclease or a CasX nuclease or a MAD7® nuclease correspond to the most frequently used codon for a particular amino acid. As to codon usage in plants, reference is made to Campbell and Gowri, 1990, Plant Physiol., 92:1-11; and Murray et al., 1989, Nucleic Acids Res., 17:477-98, each of which is incorporated herein by reference in their entireties.

In an aspect, a nucleic acid molecule encodes a guided nuclease that is codon optimized for a plant. In an aspect, a nucleic acid molecule encodes a Cas12a nuclease that is codon optimized for a plant. In an aspect, a nucleic acid molecule encodes a CasX nuclease that is codon optimized for a plant. In an aspect, a nucleic acid molecule encodes a MAD7® nuclease that is codon optimized for a plant.

In another aspect, a nucleic acid molecule provided herein encodes a guided nuclease that is codon optimized for a plant cell. In another aspect, a nucleic acid molecule provided herein encodes a guided nuclease that is codon optimized for a monocotyledonous plant species. In another aspect, a nucleic acid molecule provided herein encodes a guided nuclease that is codon optimized for a dicotyledonous plant species. In a further aspect, a nucleic acid molecule provided herein encodes a guided nuclease that is codon optimized for a gymnosperm plant species. In a further aspect, a nucleic acid molecule provided herein encodes a guided nuclease that is codon optimized for an angiosperm plant species. In a further aspect, a nucleic acid molecule provided herein encodes a guided nuclease that is codon optimized for a corn cell. In a further aspect, a nucleic acid molecule provided herein encodes a guided nuclease that is codon optimized for a soybean cell. In a further aspect, a nucleic acid molecule provided herein encodes a guided nuclease that is codon optimized for a rice cell. In a further aspect, a nucleic acid molecule provided herein encodes a guided nuclease that is codon optimized for a wheat cell. In a further aspect, a nucleic acid molecule provided herein encodes a guided nuclease that is codon optimized for a cotton cell. In a further aspect, a nucleic acid molecule provided herein encodes a guided nuclease that is codon optimized for a sorghum cell. In a further aspect, a nucleic acid molecule provided herein encodes a guided nuclease that is codon optimized for an alfalfa cell. In a further aspect, a nucleic acid molecule provided herein encodes a guided nuclease that is codon optimized for a sugarcane cell. In a further aspect, a nucleic acid molecule provided herein encodes a guided nuclease that is codon optimized for an Arabidopsis cell. In a further aspect, a nucleic acid molecule provided herein encodes a guided nuclease that is codon optimized for a tomato cell. In a further aspect, a nucleic acid molecule provided herein encodes a guided nuclease that is codon optimized for a cucumber cell. In a further aspect, a nucleic acid molecule provided herein encodes a guided nuclease that is codon optimized for a potato cell. In a further aspect, a nucleic acid molecule provided herein encodes a guided nuclease that is codon optimized for an onion cell.

In another aspect, a nucleic acid molecule provided herein encodes a Cas12a nuclease that is codon optimized for a plant cell. In another aspect, a nucleic acid molecule provided herein encodes a Cas12a nuclease that is codon optimized for a monocotyledonous plant species. In another aspect, a nucleic acid molecule provided herein encodes a Cas12a nuclease that is codon optimized for a dicotyledonous plant species. In a further aspect, a nucleic acid molecule provided herein encodes a Cas12a nuclease that is codon optimized for a gymnosperm plant species. In a further aspect, a nucleic acid molecule provided herein encodes a Cas12a nuclease that is codon optimized for an angiosperm plant species. In a further aspect, a nucleic acid molecule provided herein encodes a Cas12a nuclease that is codon optimized for a corn cell. In a further aspect, a nucleic acid molecule provided herein encodes a Cas12a nuclease that is codon optimized for a soybean cell. In a further aspect, a nucleic acid molecule provided herein encodes a Cas12a nuclease that is codon optimized for a rice cell. In a further aspect, a nucleic acid molecule provided herein encodes a Cas12a nuclease that is codon optimized for a wheat cell. In a further aspect, a nucleic acid molecule provided herein encodes a Cas12a nuclease that is codon optimized for a cotton cell. In a further aspect, a nucleic acid molecule provided herein encodes a Cas12a nuclease that is codon optimized for a sorghum cell. In a further aspect, a nucleic acid molecule provided herein encodes a Cas12a nuclease that is codon optimized for an alfalfa cell. In a further aspect, a nucleic acid molecule provided herein encodes a Cas12a nuclease that is codon optimized for a sugar cane cell. In a further aspect, a nucleic acid molecule provided herein encodes a Cas12a nuclease that is codon optimized for an Arabidopsis cell. In a further aspect, a nucleic acid molecule provided herein encodes a Cas12a nuclease that is codon optimized for a tomato cell. In a further aspect, a nucleic acid molecule provided herein encodes a Cas12a nuclease that is codon optimized for a cucumber cell. In a further aspect, a nucleic acid molecule provided herein encodes a Cas12a nuclease that is codon optimized for a potato cell. In a further aspect, a nucleic acid molecule provided herein encodes a Cas12a nuclease that is codon optimized for an onion cell.

In another aspect, a nucleic acid molecule provided herein encodes a CasX nuclease that is codon optimized for a plant cell. In another aspect, a nucleic acid molecule provided herein encodes a CasX nuclease that is codon optimized for a monocotyledonous plant species. In another aspect, a nucleic acid molecule provided herein encodes a CasX nuclease that is codon optimized for a dicotyledonous plant species. In a further aspect, a nucleic acid molecule provided herein encodes a CasX nuclease that is codon optimized for a gymnosperm plant species. In a further aspect, a nucleic acid molecule provided herein encodes a CasX nuclease that is codon optimized for an angiosperm plant species. In a further aspect, a nucleic acid molecule provided herein encodes a CasX nuclease that is codon optimized for a corn cell. In a further aspect, a nucleic acid molecule provided herein encodes a CasX nuclease that is codon optimized for a soybean cell. In a further aspect, a nucleic acid molecule provided herein encodes a CasX nuclease that is codon optimized for a rice cell. In a further aspect, a nucleic acid molecule provided herein encodes a CasX nuclease that is codon optimized for a wheat cell. In a further aspect, a nucleic acid molecule provided herein encodes a CasX nuclease that is codon optimized for a cotton cell. In a further aspect, a nucleic acid molecule provided herein encodes a CasX nuclease that is codon optimized for a sorghum cell. In a further aspect, a nucleic acid molecule provided herein encodes a CasX nuclease that is codon optimized for an alfalfa cell. In a further aspect, a nucleic acid molecule provided herein encodes a CasX nuclease that is codon optimized for a sugar cane cell. In a further aspect, a nucleic acid molecule provided herein encodes a CasX nuclease that is codon optimized for an Arabidopsis cell. In a further aspect, a nucleic acid molecule provided herein encodes a CasX nuclease that is codon optimized for a tomato cell. In a further aspect, a nucleic acid molecule provided herein encodes a CasX nuclease that is codon optimized for a cucumber cell. In a further aspect, a nucleic acid molecule provided herein encodes a CasX nuclease that is codon optimized for a potato cell. In a further aspect, a nucleic acid molecule provided herein encodes a CasX nuclease that is codon optimized for an onion cell. In another aspect, a nucleic acid molecule provided herein encodes a MAD7® nuclease that is codon optimized for a plant cell. In another aspect, a nucleic acid molecule provided herein encodes a MAD7® nuclease that is codon optimized for a monocotyledonous plant species. In another aspect, a nucleic acid molecule provided herein encodes a MAD7® nuclease that is codon optimized for a dicotyledonous plant species. In a further aspect, a nucleic acid molecule provided herein encodes a MAD7® nuclease that is codon optimized for a gymnosperm plant species. In a further aspect, a nucleic acid molecule provided herein encodes a MAD7® nuclease that is codon optimized for an angiosperm plant species. In a further aspect, a nucleic acid molecule provided herein encodes a MAD7® nuclease that is codon optimized for a corn cell. In a further aspect, a nucleic acid molecule provided herein encodes a MAD7® nuclease that is codon optimized for a soybean cell. In a further aspect, a nucleic acid molecule provided herein encodes a MAD7® nuclease that is codon optimized for a rice cell. In a further aspect, a nucleic acid molecule provided herein encodes a MAD7® nuclease that is codon optimized for a wheat cell. In a further aspect, a nucleic acid molecule provided herein encodes a MAD7® nuclease that is codon optimized for a cotton cell. In a further aspect, a nucleic acid molecule provided herein encodes a MAD7® nuclease that is codon optimized for a sorghum cell. In a further aspect, a nucleic acid molecule provided herein encodes a MAD7® nuclease that is codon optimized for an alfalfa cell. In a further aspect, a nucleic acid molecule provided herein encodes a MAD7® nuclease that is codon optimized for a sugar cane cell. In a further aspect, a nucleic acid molecule provided herein encodes a MAD7® nuclease that is codon optimized for an Arabidopsis cell. In a further aspect, a nucleic acid molecule provided herein encodes a MAD7® nuclease that is codon optimized for a tomato cell. In a further aspect, a nucleic acid molecule provided herein encodes a MAD7® nuclease that is codon optimized for a cucumber cell. In a further aspect, a nucleic acid molecule provided herein encodes a MAD7® nuclease that is codon optimized for a potato cell. In a further aspect, a nucleic acid molecule provided herein encodes a MAD7® nuclease that is codon optimized for an onion cell.

In some aspects the guided nuclease may be selected from Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Csn1, Csx12, Cas10, Csy1, Csy2, Csy3, Cse1, Csc2, 30 Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), Csf5 nuclease, Cas12c (C2c3), Cas12d (CasY), Cas12e (CasX), Cas12g, Cas12h, Cas12i, C2c4, C2c5, C2c8, C2c9, C2c10, Cas14a, Cas14b, Cas14c effector protein

In some aspects, the guided nuclease, such as a CRISPR/Cas effector protein useful with the invention may comprise a mutation in its nuclease active site (e.g., RuvC, HNH, e.g., RuvC site of a Cas12a nuclease domain, e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g., Cas9 nickase, Cas12a nickase.

In some aspects, the guided nuclease may comprise another functional domain than a nuclease, such as a adenine deaminase domain or a cytosine deaminase domain or a reverse transcriptase domain.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a polynucleotide encoding a TadA/TadA* may be codon optimized for expression in a plant.

A cytosine deaminase catalyzes cytosine deamination and results in a thymidine (through a uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “−”, complementary) strand of the target nucleic acid.

The nucleic acid constructs of the invention encoding a base editor comprising a sequence-specific DNA binding protein and a cytosine deaminase polypeptide, and nucleic acid constructs/expression cassettes/vectors encoding the same, may be used in combination with guide nucleic acids for modifying target nucleic acid including, but not limited to, generation of C→T or G→A mutations in a target nucleic acid including, but not limited to, a plasmid sequence; generation of C→T or G+A mutations in a coding sequence to alter an amino acid identity; generation of C→T or G→A mutations in a coding sequence to generate a stop codon; generation of C→T or G→A mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention encoding a base editor comprising a sequence-specific DNA binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt function; and/or generation of point mutations in genomic DNA to disrupt splice junctions.

### Target Sites

As used herein, a “target sequence” refers to a selected sequence or region of a DNA molecule in which a modification (e.g., cleavage, site-directed integration) is desired. A target sequence comprises a target site.

As used herein, a “target site” refers to the portion of a target sequence that is cleaved by a guided nuclease such as CRISPR nuclease. In contrast to a non-target nucleic acid (e.g., non-target ssDNA) or non-target region, a target site comprises significant complementarity to a guide nucleic acid or a guide RNA.

In an aspect, a target site is 100% complementary to a guide nucleic acid. In another aspect, a target site is 99% complementary to a guide nucleic acid. In another aspect, a target site is 98% complementary to a guide nucleic acid. In another aspect, a target site is 97% complementary to a guide nucleic acid. In another aspect, a target site is 96% complementary to a guide nucleic acid. In another aspect, a target site is 95% complementary to a guide nucleic acid. In another aspect, a target site is 94% complementary to a guide nucleic acid. In another aspect, a target site is 93% complementary to a guide nucleic acid. In another aspect, a target site is 92% complementary to a guide nucleic acid. In another aspect, a target site is 91% complementary to a guide nucleic acid. In another aspect, a target site is 90% complementary to a guide nucleic acid. In another aspect, a target site is 85% complementary to a guide nucleic acid. In another aspect, a target site is 80% complementary to a guide nucleic acid.

In an aspect, a target site comprises at least one PAM site. In an aspect, a target site is adjacent to a nucleic acid sequence that comprises at least one PAM site. In another aspect, a target site is within 5 nucleotides of at least one PAM site. In a further aspect, a target site is within 10 nucleotides of at least one PAM site. In another aspect, a target site is within 15 nucleotides of at least one PAM site. In another aspect, a target site is within 20 nucleotides of at least one PAM site. In another aspect, a target site is within 25 nucleotides of at least one PAM site. In another aspect, a target site is within 30 nucleotides of at least one PAM site.

In an aspect, a target site is positioned within genic DNA. In another aspect, a target site is positioned within a gene. In another aspect, a target site is positioned within a gene of interest. In another aspect, a target site is positioned within an exon of a gene. In another aspect, a target site is positioned within an intron of a gene. In another aspect, a target site is positioned within the promoter of a gene. In another aspect, a target site is positioned within 5′-UTR of a gene. In another aspect, a target site is positioned within a 3′-UTR of a gene. In another aspect, a target site is positioned within intergenic DNA.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

```
    5′-NNNNNNNNNNNNNNNNNNN-3′ RNA Spacer
          |||||||||||||||||||
3′AAANNNNNNNNNNNNNNNNNNN-5′ Target strand
    |||
5′TTTNNNNNNNNNNNNNNNNNNN-3′ Non-target strand
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems ((2015) Nature Reviews Microbiology 13:722-736). Guide structures and PAMs are described in by R. Barrangou ((2015) Genome Biol. 16:247).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. (2013) Nat. Methods 10:1116-1121; Jiang et al. (2013) Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. (2014) Appl. Environ. Microbiol. 80:994-1001; Mojica et al. (2009) Microbiology 155:733-740).

In an aspect, a target DNA molecule is single-stranded. In another aspect, a target DNA molecule is double-stranded.

In an aspect, a target sequence comprises genomic DNA. In an aspect, a target sequence is positioned within a nuclear genome. In an aspect, a target sequence comprises chromosomal DNA. In an aspect, a target sequence comprises plasmid DNA. In an aspect, a target sequence is positioned within a plasmid. In an aspect, a target sequence comprises mitochondrial DNA. In an aspect, a target sequence is positioned within a mitochondrial genome. In an aspect, a target sequence comprises plastid DNA. In an aspect, a target sequence is positioned within a plastid genome. In an aspect, a target sequence comprises chloroplast DNA. In an aspect, a target sequence is positioned within a chloroplast genome. In an aspect, a target sequence is positioned within a genome selected from the group consisting of a nuclear genome, a mitochondrial genome, and a plastid genome.

In an aspect, a target sequence comprises genic DNA. As used herein, “genic DNA” refers to DNA that encodes one or more genes. In another aspect, a target sequence comprises intergenic DNA. In contrast to genic DNA, “intergenic DNA” comprises noncoding DNA, and lacks DNA encoding a gene. In an aspect, intergenic DNA is positioned between two genes.

In an aspect, a target sequence encodes a gene. As used herein, a “gene” refers to a polynucleotide that can produce a functional unit (e.g., without being limiting, for example, a protein, or a non-coding RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a non-protein-coding RNA molecule or a precursor thereof. In another aspect, a gene encodes a protein. In some embodiments, the target sequence is selected from the group consisting of: a promoter, an enhancer sequence, a leader sequence, a transcriptional start site, a transcriptional stop site, a polyadenylation site, an exon, an intron, a splice site, a 5′-UTR, a 3′-UTR, a protein coding sequence, a non-protein-coding sequence, a miRNA, a pre-miRNA and a miRNA binding site.

Non-limiting examples of a non-protein-coding RNA molecule include a microRNA (miRNA), a miRNA precursor (pre-miRNA), a small interfering RNA (siRNA), a small RNA (18 to 26 nucleotides in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracrRNA), a guide RNA (gRNA), and a single guide RNA (sgRNA). In an aspect, a non-protein-coding RNA molecule comprises a miRNA. In an aspect, a non-protein-coding RNA molecule comprises a siRNA. In an aspect, a non-protein-coding RNA molecule comprises a ta-siRNA. In an aspect, a non-protein-coding RNA molecule is selected from the group consisting of a miRNA, a siRNA, and a ta-siRNA.

As used herein, a “gene of interest” refers to a polynucleotide sequence encoding a protein or a non-protein-coding RNA molecule that is to be integrated into a target sequence, or, alternatively, an endogenous polynucleotide sequence encoding a protein or a non-protein-coding RNA molecule that is to be edited by a ribonucleoprotein. In an aspect, a gene of interest encodes a protein. In another aspect, a gene of interest encodes a non-protein-coding RNA molecule. In an aspect, a gene of interest is exogenous to a targeted DNA molecule. In an aspect, a gene of interest replaces an endogenous gene in a targeted DNA molecule.

### Mutations

In an aspect, a ribonucleoprotein or method provided herein generates at least one mutation in a target sequence.

In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to a floral cell-preferred promoter or a second nucleic acid encoding at least one guide nucleic acid operably linked to a heterologous second promoter. In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to a floral tissue-preferred promoter or a second nucleic acid encoding at least one guide nucleic acid operably linked to a heterologous second promoter.

In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to a heterologous promoter or a second nucleic acid encoding at least one guide nucleic acid operably linked to a floral cell-preferred promoter. In an aspect, a seed produced from a plant provided herein comprises at least one mutation in a gene of interest comprising a target site as compared to a seed of a control plant of the same line or variety that lacks a first nucleic acid sequence encoding a guided nuclease operably linked to a heterologous promoter or a second nucleic acid encoding at least one guide nucleic acid operably linked to a floral tissue-preferred promoter.

As used herein, a “mutation” refers to a non-naturally occurring alteration to a nucleic acid or amino acid sequence as compared to a naturally occurring reference nucleic acid or amino acid sequence from the same organism. It will be appreciated that, when identifying a mutation, the reference sequence should be from the same nucleic acid (e.g, gene, non-coding RNA) or amino acid (e.g, protein). In determining if a difference between two sequences comprises a mutation, it will be appreciated in the art that the comparison should not be made between homologous sequences of two different species or between homologous sequences of two different varieties of a single species. Rather, the comparison should be made between the edited (e.g., mutated) sequence and the endogenous, non-edited (e.g., “wildtype”) sequence of the same organism.

Several types of mutations are known in the art. In an aspect, a mutation comprises an insertion. An “insertion” refers to the addition of one or more nucleotides or amino acids to a given polynucleotide or amino acid sequence, respectively, as compared to an endogenous reference polynucleotide or amino acid sequence. In another aspect, a mutation comprises a deletion. A “deletion” refers to the removal of one or more nucleotides or amino acids to a given polynucleotide or amino acid sequence, respectively, as compared to an endogenous reference polynucleotide or amino acid sequence. In another aspect, a mutation comprises a substitution. A “substitution” refers to the replacement of one or more nucleotides or amino acids to a given polynucleotide or amino acid sequence, respectively, as compared to an endogenous reference polynucleotide or amino acid sequence. In another aspect, a mutation comprises an inversion. An “inversion” refers to when a segment of a polynucleotide or amino acid sequence is reversed end-to-end. In an aspect, a mutation provided herein comprises a mutation selected from the group consisting of an insertion, a deletion, a substitution, and an inversion.

In an aspect, a plant or seed comprises at least one mutation in a gene of interest, where the at least one mutation results in the deletion of one or more amino acids from a protein encoded by the gene of interest as compared to a wildtype protein.

In an aspect, a plant or seed comprises at least one mutation in a gene of interest, where the at least one mutation results in the substitution of one or more amino acids within a protein encoded by the gene of interest as compared to a wildtype protein.

In an aspect, a plant or seed comprises at least one mutation in a gene of interest, where the at least one mutation results in the insertion of one or more amino acids within a protein encoded by the gene of interest as compared to a wildtype protein.

Mutations in coding regions of genes (e.g., exonic mutations) can result in a truncated protein or polypeptide when a mutated messenger RNA (mRNA) is translated into a protein or polypeptide. In an aspect, this disclosure provides a mutation that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids.

Without being limited by any scientific theory, one way to cause a protein or polypeptide truncation is by the introduction of a premature stop codon in an mRNA transcript of an endogenous gene. In an aspect, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

In an aspect, a seed or plant comprises at least one mutation, where the at least one mutation results in the introduction of a premature stop codon in a messenger RNA encoded by the gene of interest as compared to a wildtype messenger RNA.

In an aspect, a mutation provided herein comprises a null mutation. As used herein, a “null mutation” refers to a mutation that confers a complete loss-of-function for a protein encoded by a gene comprising the mutation, or, alternatively, a mutation that confers a complete loss-of-function for a small RNA encoded by a genomic locus. A null mutation can cause lack of mRNA transcript production, a lack of small RNA transcript production, a lack of protein function, or a combination thereof.

A mutation provided herein can be positioned in any part of an endogenous gene. In an aspect, a mutation provided herein is positioned within an exon of an endogenous gene. In another aspect, a mutation provided herein is positioned within an intron of an endogenous gene. In a further aspect, a mutation provided herein is positioned within a 5′-untranslated region of an endogenous gene. In still another aspect, a mutation provided herein is positioned within a 3′-untranslated region of an endogenous gene. In yet another aspect, a mutation provided herein is positioned within a promoter of an endogenous gene.

In an aspect, a mutation is positioned at a splice site within a gene. A mutation at a splice site can interfere with the splicing of exons during mRNA processing. If one or more nucleotides are inserted, deleted, or substituted at a splice site, splicing can be perturbed. Perturbed splicing can result in unspliced introns, missing exons, or both, from a mature mRNA sequence. Typically, although not always, a “GU” sequence is required at the 5′ end of an intron and a “AG” sequence is required at the 3′ end of an intron for proper splicing. If either of these splice sites are mutated, splicing perturbations can occur.

In an aspect, a seed or plant comprises at least one mutation, where the at least one mutation comprises the deletion of one or more splice sites from a gene of interest. In another aspect, a seed or plant comprises at least one mutation, where the at least one mutation is positioned within one or more splice sites from a gene of interest.

In an aspect, a mutation comprises a site-directed integration. In an aspect, a site-directed integration comprises the insertion of all or part of a desired sequence into a target sequence.

As used herein, “site-directed integration” refers to all, or a portion, of a desired sequence (e.g., an exogenous gene, an edited endogenous gene) being inserted or integrated at a desired site or locus within the plant genome (e.g., target sequence). As used herein, a “desired sequence” refers to a DNA molecule comprising a nucleic acid sequence that is to be integrated into a genome of a plant or plant cell. The desired sequence can comprise a transgene or construct. In an aspect, a nucleic acid molecule comprising a desired sequence comprises one or two homology arms flanking the desired sequence to promote the targeted insertion event through homologous recombination and/or homology-directed repair.

In an aspect, a method provided herein comprises site-directed integration of a desired sequence into a target sequence.

Any site or locus within the genome of a plant can be chosen for site-directed integration of a transgene or construct of the present disclosure. In an aspect, a target sequence is positioned within a B, or supernumerary, chromosome.

For site-directed integration, a double-strand break (DSB) or nick may first be made at a target sequence via a guided nuclease or ribonucleoprotein provided herein. In the presence of a desired sequence, the DSB or nick can then be repaired by homologous recombination (HR) between the homology arm(s) of the desired sequence and the target sequence, or by non-homologous end joining (NHEJ), resulting in site-directed integration of all or part of the desired sequence into the target sequence to create the targeted insertion event at the site of the DSB or nick.

In an aspect, site-directed integration comprises the use of NHEJ repair mechanisms endogenous to a cell. In another aspect, site-directed integration comprises the use of HR repair mechanisms endogenous to a cell.

In an aspect, repair of a double-stranded break generates at least one mutation in a gene of interest as compared to a control plant of the same line or variety.

In an aspect, a mutation comprises the integration of at least 5 contiguous nucleotides of a desired sequence into a target sequence. In an aspect, a mutation comprises the integration of at least 10 contiguous nucleotides of a desired sequence molecule into a target sequence. In an aspect, a mutation comprises the integration of at least 15 contiguous nucleotides of a desired sequence into a target sequence. In an aspect, a mutation comprises the integration of at least 20 contiguous nucleotides of a desired sequence into a target sequence. In an aspect, a mutation comprises the integration of at least 25 contiguous nucleotides of a desired sequence into a target sequence. In an aspect, a mutation comprises the integration of at least 50 contiguous nucleotides of a desired sequence into a target sequence. In an aspect, a mutation comprises the integration of at least 100 contiguous nucleotides of a desired sequence into a target sequence. In an aspect, a mutation comprises the integration of at least 250 contiguous nucleotides of a desired sequence into a target sequence. In an aspect, a mutation comprises the integration of at least 500 contiguous nucleotides of a desired sequence into a target sequence. In an aspect, a mutation comprises the integration of at least 1000 contiguous nucleotides of a desired sequence into a target sequence. In an aspect, a mutation comprises the integration of at least 2000 contiguous nucleotides of a desired sequence into a target sequence.

In an aspect, a mutation comprises the integration of between 5 contiguous nucleotides and 3500 contiguous nucleotides of a desired sequence into a target sequence. In an aspect, a mutation comprises the integration of between 5 contiguous nucleotides and 2500 contiguous nucleotides of a desired sequence into a target sequence. In an aspect, a mutation comprises the integration of between 5 contiguous nucleotides and 1500 contiguous nucleotides of a desired sequence into a target sequence. In an aspect, a mutation comprises the integration of between 5 contiguous nucleotides and 750 contiguous nucleotides of a desired sequence into a target sequence. In an aspect, a mutation comprises the integration of between 5 contiguous nucleotides and 500 contiguous nucleotides of a desired sequence into a target sequence. In an aspect, a mutation comprises the integration of between 5 contiguous nucleotides and 250 contiguous nucleotides of a desired sequence into a target sequence. In an aspect, a mutation comprises the integration of between 5 contiguous nucleotides and 150 contiguous nucleotides of a desired sequence into a target sequence. In an aspect, a mutation comprises the integration of between 25 contiguous nucleotides and 2500 contiguous nucleotides of a desired sequence into a target sequence. In an aspect, a mutation comprises the integration of between 25 contiguous nucleotides and 1500 contiguous nucleotides of a desired sequence into a target sequence. In an aspect, a mutation comprises the integration of between 25 contiguous nucleotides and 750 contiguous nucleotides of a desired sequence into a target sequence. In an aspect, a mutation comprises the integration of between 50 contiguous nucleotides and 2500 contiguous nucleotides of a desired sequence into a target sequence. In an aspect, a mutation comprises the integration of between 50 contiguous nucleotides and 1500 contiguous nucleotides of a desired sequence into a target sequence. In an aspect, a mutation comprises the integration of between 50 contiguous nucleotides and 750 contiguous nucleotides of a desired sequence into a target sequence. In an aspect, a mutation comprises the integration of between 100 contiguous nucleotides and 2500 contiguous nucleotides of a desired sequence into a target Sequence. In an aspect, a mutation comprises the integration of between 100 contiguous nucleotides and 1500 contiguous nucleotides of a desired sequence into a target Sequence. In an aspect, a mutation comprises the integration of between 100 contiguous nucleotides and 750 contiguous nucleotides of a desired sequence into a target Sequence.

In an aspect, a method provided herein further comprises detecting an edit or a mutation in a target sequence. The screening and selection of mutagenized or edited plants or plant cells can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454) enzymatic assays for detecting enzyme or ribozyme activity of polypeptides and polynucleotides, protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the above-referenced techniques are known in the art.

In an aspect, a sequence provided herein encodes at least one ribozyme. In an aspect, a sequence provided herein encodes at least two ribozymes. In an aspect, a ribozyme is a self-cleaving ribozyme. Self-cleaving ribozymes are known in the art. For example, see Jimenez et al., Trends Biochem. Sci., 40:648-661 (2015).

In an aspect, a sequence encoding at least one guide nucleic acid is flanked by self-cleaving ribozymes. In an aspect, a sequence encoding at least one guide nucleic acid is immediately adjacent to a sequence encoding a ribozyme (e.g., the 5′-most nucleotide of the guide nucleic acid abuts the 3′-most nucleotide of the ribozyme or the 3′-most nucleotide of the guide nucleic acid abuts the 5′-most nucleotide of the ribozyme). In an aspect, a sequence encoding at least one guide nucleic acid is separated from a sequence encoding a ribozyme by at least 1, at least 2, at least 3, at least 4, at least 5, at least 10, at least 20, at least 30, at least 40, at least 50, at least 100, at least 250, at least 500, or at least 10000 nucleotides.

### Plants

Any plant or plant cell can be used with the methods and compositions provided herein. In an aspect, a plant is selected from the group consisting of a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, and a potato plant. In an aspect, a plant is an angiosperm. In an aspect, a plant is a gymnosperm. In an aspect, a plant is a monocotyledonous plant. In an aspect, a plant is a dicotyledonous plant. In an aspect, a plant is a plant of a family selected from the group consisting of Alliaceae, Anacardiaceae, Apiaceae, Arecaceae, Asteraceae, Brassicaceae, Caesalpiniaceae, Cucurbitaceae, Ericaceae, Fabaceae, Juglandaceae, Malvaceae, Mimosaceae, Moraceae, Musaceae, Orchidaceae, Papilionaceae, Pinaceae, Poaceae, Rosaceae, Rutaceae, Rubiaceae, and Solanaceae. In an aspect, a plant cell is selected from the group consisting of a corn cell, a rice cell, a sorghum cell, a wheat cell, an alfalfa cell, a barley cell, a millet cell, a rye cell, a sugarcane cell, a cotton cell, a soybean cell, a canola cell, a tomato cell, an onion cell, a cucumber cell, an Arabidopsis cell, and a potato cell. In an aspect, a plant cell is an angiosperm plant cell. In an aspect, a plant cell is a gymnosperm plant cell. In an aspect, a plant cell is a monocotyledonous plant cell. In an aspect, a plant cell is a dicotyledonous plant cell. In an aspect, a plant cell is a plant cell of a family selected from the group consisting of Alliaceae, Anacardiaceae, Apiaceae, Arecaceae, Asteraceae, Brassicaceae, Caesalpiniaceae, Cucurbitaceae, Ericaceae, Fabaceae, Juglandaceae, Malvaceae, Mimosaceae, Moraceae, Musaceae, Orchidaceae, Papilionaceae, Pinaceae, Poaceae, Rosaceae, Rutaceae, Rubiaceae, and Solanaceae.

As used herein, a “variety” refers to a group of plants within a species (e.g., without being limiting Zea mays) that share certain genetic traits that separate them from other possible varieties within that species. Varieties can be inbreds or hybrids, though commercial plants are often hybrids to take advantage of hybrid vigor. Individuals within a hybrid cultivar are homogeneous, nearly genetically identical, with most loci in the heterozygous state.

As used herein, the term “inbred” means a line that has been bred for genetic homogeneity. In an aspect, a seed provided herein is an inbred seed. In an aspect, a plant provided herein is an inbred plant.

As used herein, the term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines. In an aspect, a seed provided herein is a hybrid seed. In an aspect, a plant provided herein is a hybrid plant.

In some jurisdictions, products obtained exclusively by essentially biological processes, such as plant products are excluded from patent protection. Accordingly, the claimed plants, plant parts and cells and their progeny can be defined as directed only to those plants, plant parts and cells and their progeny which are obtained by technical intervention (regardless of any further propagation through crossing and selection). An embodiment of the invention is directed at plants, or plant parts or progeny produced or obtainable using gene editing technology herein described. Alternatively, the subject matter excluded from patentability may be disclaimed. An embodiment of the invention is directed at plants, part of plants or progeny thereof comprising the genomic alterations as elsewhere herein described, provided that the plants, parts or plants or progeny are not obtained exclusively through essentially biological processes, wherein essentially biological processes are processes for the production of plants or animals if they consist entirely of natural phenomena such as crossing or selection.

### Transformation

Methods can involve transient transformation or stable integration of any nucleic acid molecule into any plant or plant cell provided herein.

As used herein, “stable integration” or “stably integrated” refers to a transfer of DNA into genomic DNA of a targeted cell or plant that allows the targeted cell or plant to pass the transferred DNA to the next generation of the transformed organism. Stable transformation requires the integration of transferred DNA within the reproductive cell(s) of the transformed organism. As used herein, “transiently transformed” or “transient transformation” refers to a transfer of DNA into a cell that is not transferred to the next generation of the transformed organism. In a transient transformation the transformed DNA does not typically integrate into the transformed cell's genomic DNA. In one aspect, a method stably transforms a plant cell or plant with one or more nucleic acid molecules provided herein. In another aspect, a method transiently transforms a plant cell or plant with one or more nucleic acid molecules provided herein.

In an aspect, a nucleic acid molecule encoding a guided nuclease is stably integrated into a genome of a plant. In an aspect, a nucleic acid molecule encoding a Cas12a nuclease is stably integrated into a genome of a plant. In an aspect, a nucleic acid molecule encoding a CasX nuclease is stably integrated into a genome of a plant. In an aspect, a nucleic acid molecule encoding a guide nucleic acid is stably integrated into a genome of a plant. In an aspect, a nucleic acid molecule encoding a guide RNA is stably integrated into a genome of a plant. In an aspect, a nucleic acid molecule encoding a single-guide RNA is stably integrated into a genome of a plant.

Numerous methods for transforming cells with a recombinant nucleic acid molecule or construct are known in the art, which can be used according to methods of the present application. Any suitable method or technique for transformation of a cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants.

In an aspect, a method comprises providing a cell with a nucleic acid molecule via Agrobacterium-mediated transformation. In an aspect, a method comprises providing a cell with a nucleic acid molecule via polyethylene glycol-mediated transformation. In an aspect, a method comprises providing a cell with a nucleic acid molecule via biolistic transformation. In an aspect, a method comprises providing a cell with a nucleic acid molecule via liposome-mediated transfection. In an aspect, a method comprises providing a cell with a nucleic acid molecule via viral transduction. In an aspect, a method comprises providing a cell with a nucleic acid molecule via use of one or more delivery particles. In an aspect, a method comprises providing a cell with a nucleic acid molecule via microinjection. In an aspect, a method comprises providing a cell with a nucleic acid molecule via electroporation.

In an aspect, a nucleic acid molecule is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

Other methods for transformation, such as vacuum infiltration, pressure, sonication, and silicon carbide fiber agitation, are also known in the art and envisioned for use with any method provided herein.

Methods of transforming cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell with any of the nucleic acid molecules provided herein.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

Delivery vehicles, vectors, particles, nanoparticles, formulations and components thereof for expression of one or more elements of a nucleic acid molecule are as used in WO 2014/093622. In an aspect, a method of providing a nucleic acid molecule or a protein to a cell comprises delivery via a delivery particle. In an aspect, a method of providing a nucleic acid molecule to a plant cell or plant comprises delivery via a delivery vesicle. In an aspect, a delivery vesicle is selected from the group consisting of an exosome and a liposome. In an aspect, a method of providing a nucleic acid molecule to a plant cell or plant comprises delivery via a viral vector. In an aspect, a viral vector is selected from the group consisting of an adenovirus vector, a lentivirus vector, and an adeno-associated viral vector. In another aspect, a method providing a nucleic acid molecule to a plant cell or plant comprises delivery via a nanoparticle. In an aspect, a method providing a nucleic acid molecule to a plant cell or plant comprises microinjection. In an aspect, a method providing a nucleic acid molecule to a plant cell or plant comprises polycations. In an aspect, a method providing a nucleic acid molecule to a plant cell or plant comprises a cationic oligopeptide.

In an aspect, a delivery particle is selected from the group consisting of an exosome, an adenovirus vector, a lentivirus vector, an adeno-associated viral vector, a nanoparticle, a polycation, and a cationic oligopeptide. In an aspect, a method provided herein comprises the use of one or more delivery particles. In another aspect, a method provided herein comprises the use of two or more delivery particles. In another aspect, a method provided herein comprises the use of three or more delivery particles.

Suitable agents to facilitate transfer of nucleic acids into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning includes (a) surfactants, (b) organic solvents, aqueous solutions, or aqueous mixtures of organic solvents, (c) oxidizing agents, (e) acids. (f) bases, (g) oils, (h) enzymes, or combinations thereof.

Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e. g., plant-sourced oils, crop oils (such as those listed in the 9^{th} Compendium of Herbicide Adjuvants, publicly available on line at www(dot)herbicide(dot)adjuvants(dot)com) can be used, e. g., paraffinic oils, polyol fatty acid esters, or oils with short-chain molecules modified with amides or polyamines such as polyethyleneimine or N-pyrrolidine.

Examples of useful surfactants include sodium or lithium salts of fatty acids (such as tallow or tallowamines or phospholipids) and organosilicone surfactants. Other useful surfactants include organosilicone surfactants including nonionic organosilicone surfactants, e. g., trisiloxane ethoxylate surfactants or a silicone polyether copolymer such as a copolymer of polyalkylene oxide modified heptamethyl trisiloxane and allyloxypolypropylene glycol methylether (commercially available as Silwet® L-77).

Useful physical agents can include (a) abrasives such as carborundum, corundum, sand, calcite, pumice, garnet, and the like, (b) nanoparticles such as carbon nanotubes or (c) a physical force. Carbon nanotubes are disclosed by Kam et. al. (2004) Am. Chem. Soc, 126 (22): 6850-6851, Liu et. al. (2009) Nano Lett, 9 (3): 1007-1010, and Khodakovskaya et. al. (2009) ACS Nano, 3 (10): 3221-3227. Physical force agents can include heating, chilling, the application of positive pressure, or ultrasound treatment. Embodiments of the method can optionally include an incubation step, a neutralization step (e.g., to neutralize an acid, base, or oxidizing agent, or to inactivate an enzyme), a rinsing step, or combinations thereof. The methods of the invention can further include the application of other agents which will have enhanced effect due to the silencing of certain genes. For example, when a polynucleotide is designed to regulate genes that provide herbicide resistance, the subsequent application of the herbicide can have a dramatic effect on herbicide efficacy.

Agents for laboratory conditioning of a plant cell to permeation by polynucleotides include, e.g., application of a chemical agent, enzymatic treatment, heating or chilling, treatment with positive or negative pressure, or ultrasound treatment. Agents for conditioning plants in a field include chemical agents such as surfactants and salts.

In an aspect, a transformed or transfected cell is a plant cell. Recipient plant cell or explant targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, or a vascular tissue cell. In another aspect, this disclosure provides a plant chloroplast. In a further aspect, this disclosure provides an epidermal cell, a guard cell, a trichome cell, a root hair cell, a storage root cell, or a tuber cell. In another aspect, this disclosure provides a protoplast. In another aspect, this disclosure provides a plant callus cell. Any cell from which a fertile plant can be regenerated is contemplated as a useful recipient cell for practice of this disclosure. Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U.S. Patent Application Publication 2004/0216189, all of which are incorporated herein by reference. Transformed explants, cells or tissues can be subjected to additional culturing steps, such as callus induction, selection, regeneration, etc., as known in the art. Transformed cells, tissues or explants containing a recombinant DNA insertion can be grown, developed or regenerated into transgenic plants in culture, plugs or soil according to methods known in the art. In one aspect, this disclosure provides plant cells that are not reproductive material and do not mediate the natural reproduction of the plant. In another aspect, this disclosure also provides plant cells that are reproductive material and mediate the natural reproduction of the plant. In another aspect, this disclosure provides plant cells that cannot maintain themselves via photosynthesis. In another aspect, this disclosure provides somatic plant cells. Somatic cells, contrary to germline cells, do not mediate plant reproduction. In one aspect, this disclosure provides a non-reproductive plant cell.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following examples are included to demonstrate embodiments of the disclosure. It should be appreciated by those of skill in the art that many changes can be made in the specific embodiments which are disclosed and still obtain a like or similar result without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 1

Guide RNA Titration Assay to Evaluate gRNA Expression Strategies Using Transfected Protoplasts

The aim of this study was to design a CRISPR/Cas12a guide RNA titration assay to evaluate expression strategies for guide RNAs of varying editing efficiencies. Two approaches were tested: (1) using multiple gRNA cassettes each driven by separate promoters or (2) duplicating the same guide RNA unit in the single gRNA array driven by a single promoter.

As a first step, two independent maize target sequences for gRNAs were selected using a gRNA design computational algorithm described in US20230091138 and incorporated here in its entirety (see Table 1). The gRNA spacer SP1 (SEQ ID NO: 7) targets the maize GA20 oxidase_3 gene (GA200x_3; SEQ ID NO: 50) and is a gRNA with high editing efficiency. It has a predicted editing efficiency score of 2.25 as per the gRNA design computational algorithm and an observed editing rate of 88.3% . . . . The gRNA spacer SP2 (SEQ ID NO: 8) targets the maize Brown midrib 3 gene (Bmr3; SEQ ID NO: 51) and is a guide RNA with moderate editing efficiency. The guide RNA has a predicted editing efficiency score of 0 as per the gRNA design computational algorithm and an observed editing rate of 31.6%, indicating that it has moderate editing efficiency.

TABLE 1
gRNA spacer sequences and predicted Edit scores for each gRNA.

| | | | | Predicted | Observed |
|---|---|---|---|---|---|
| | Spacer | | Spacer | Edit | editing |
| gRNA target | name | Spacer sequence | SEQ ID NO | score | rate |
| GA20ox_3 | SP1 | GACGACCCTACTGCTACTACTAC | 7 | 2.25 | 88.3% |
| Bmr3 | SP2 | CGGCAGCGCGTCGTAGCAGTTCT | 8 | 0 | 31.6% |

Seven Cas12a and gRNA-expressing vectors were designed to generate targeted mutations within the GA20 oxidase_3 gene. Each vector was designed so as to vary guide RNA expression while keeping the Cas12a levels constant. Each vector had a functional cassette for the expression of Cas12a (also known as Cpf1) comprising a rice actin constitutive promoter, leader, and intron, P-Os.Act (SEQ ID NO: 1), operably linked 5′ to a plant codon optimized sequence for Lachnospiraceae bacterium Cas12a RNA-guided endonuclease (SEQ ID NO: 2). The protein sequence of LbCas12a is set forth as SEQ ID NO:4. The LbCas12a DNA sequence was flanked by DNA sequences encoding nuclear localization signal (NLS) sequences at the 5′ and 3′ ends (SEQ ID: 48 and SEQ ID: 49) and operably linked 5′ to a transcription terminator sequence from a rice Lipid transfer protein (LTP) gene (SEQ ID NO:3). Each guide RNA cassette comprised a common Direct repeat sequence (DR) (SEQ ID NO: 9) compatible with the Cas12a enzyme and the SP1 spacer/targeting sequence complementary to its intended target site on the GA20 oxidase_3 gene. Each of the seven vectors varied in either the number of unique gRNA cassettes each driven by a unique promoter or in the number of spacer elements within each gRNA cassette.

As shown in Table 2, pM325 comprised a single guide RNA cassette comprising a single copy of the spacer SP1. The cassette comprised a synthetic RNA Polymerase III (Pol III) promoter GSP2262 (SEQ ID NO: 10) operably linked to a transcribable sequence comprising, in order: a Cas12a-compatible Direct repeat (DR) sequence (SEQ ID NO:9), spacer SP1 (SEQ ID NO:7), a DR and a poly (T)₇ terminator.

pM327 comprised a single guide RNA cassette comprising an array of four copies of the spacer SP1. The cassette comprised the synthetic Pol III promoter GSP2262 (SEQ ID NO: 10) operably linked to a transcribable sequence comprising, in order: a Cas12a-compatible Direct repeat (DR) sequence (SEQ ID NO:9), spacer SP1 (SEQ ID NO:7), a DR, SP1, DR, SP1, DR, SP1, DR and a poly (T)₇ terminator. The DR-SP1-DR-SP1-DR-SP1-DR-SP1-DR portion of the transcript is a pre-crRNA precursor RNA that can be processed by Cas12a into four copies of mature SP1 guide RNAs.

pM328 comprised a single guide RNA cassette comprising an array of eight copies of the spacer SP1. The cassette comprised the synthetic promoter GSP2262 (SEQ ID NO: 10) operably linked to a transcribable sequence comprising, in order: a Cas12a-compatible Direct repeat (DR) sequence (SEQ ID NO:9), spacer SP1 (SEQ ID NO:7), DR, SP1, DR, SP1, DR, SP1, DR, SP1, DR, SP1, DR, SP1 and a DR and a poly (T)₇ terminator. The DR-SP1-DR-SP1-DR-SP1-DR-SP1-DR-SP1-DR-SP1-DR-SP1-DR-SP1-DR portion of the transcript is a pre-crRNA precursor RNA that can be processed by Cas12a into eight copies of mature SP1 guide RNAs.

pM329 comprised two guide RNA cassettes each driven by a unique Pol III promoter and comprising a single copy of the spacer SP1. gRNA cassette 1 comprised a synthetic Pol III promoter GSP2262 (SEQ ID NO: 10) operably linked to a transcribable sequence comprising, in order: a Cas12a-compatible Direct repeat (DR) sequence (SEQ ID NO:9), spacer SP1 (SEQ ID NO: 7), DR and a poly (T)₇ terminator. gRNA cassette 2 comprised a synthetic Pol III promoter GSP2273 (SEQ ID NO: 11) operably linked to a transcribable sequence comprising, in order: a DR sequence (SEQ ID NO:9), spacer SP1 (SEQ ID NO:7), DR and a poly (T)₇ terminator.

pM330 comprised two guide RNA cassettes each comprising an array of four copies of the spacer SP1. gRNA cassette 1 comprised the synthetic Pol III promoter GSP2262 (SEQ ID NO: 10) operably linked to a transcribable sequence comprising, in order: a Cas12a-compatible Direct repeat (DR) sequence (SEQ ID NO:9), spacer SP1 (SEQ ID NO:7), a DR, SP1, DR, SP1, DR, SP1, DR and a poly (T)₇ terminator. gRNA cassette 2 comprised a synthetic Pol III promoter GSP2273 (SEQ ID NO: 11) operably linked to a transcribable sequence comprising, in order: a DR (SEQ ID NO:9), spacer SP1 (SEQ ID NO:7), a DR, SP1, DR, SP1, DR, SP1, DR and a poly (T)₇ terminator. For each gRNA transcript array, the DR-SP1-DR-SP1-DR-SP1-DR-SP1-DR portion of the transcript is a pre-crRNA precursor RNA that can be processed by Cas12a into four copies of mature SP1 guide RNAs.

pM331 comprised four guide RNA cassettes each driven by a unique Pol III promoter and comprising a single copy of the spacer SP1. gRNA cassette 1 comprised a synthetic Pol III promoter GSP2262 (SEQ ID NO: 10) operably linked to a transcribable sequence comprising, in order: a Cas12a-compatible Direct repeat (DR) sequence (SEQ ID NO:9), spacer SP1 (SEQ ID NO: 7), DR and a poly (T)₇ terminator. gRNA cassette 2 comprised a synthetic Pol III promoter GSP2273 (SEQ ID NO: 11) operably linked to a transcribable sequence comprising, in order: a DR (SEQ ID NO:9), spacer SP1 (SEQ ID NO:7), DR and a poly (T)₇ terminator. gRNA cassette 3 comprised a synthetic Pol III promoter GSP2239 (SEQ ID NO: 12) operably linked to a transcribable sequence comprising, in order: a DR (SEQ ID NO:9), a spacer SP1 (SEQ ID NO:7), DR and a poly (T)₇ terminator. gRNA cassette 4 comprised a synthetic Pol III promoter GSP2244 (SEQ ID NO: 13) operably linked to a transcribable sequence comprising, in order: a DR (SEQ ID NO: 9), a spacer SP1 (SEQ ID NO:7), DR and a poly (T)₇ terminator.

pM332 comprised four guide RNA cassettes each comprising two copies of the spacer SP1. gRNA cassette 1 comprised the synthetic Pol III promoter GSP2262 (SEQ ID NO: 10) operably linked to a transcribable sequence comprising, in order: a Cas12a-compatible Direct repeat (DR) sequence (SEQ ID NO:9), spacer SP1 (SEQ ID NO:7), a DR, SP1, and a poly (T)₇ terminator. gRNA cassette 2 comprised a synthetic Pol III promoter GSP2273 (SEQ ID NO: 11) operably linked to a transcribable sequence comprising, in order: a DR (SEQ ID NO:9), spacer SP1 (SEQ ID NO: 7), a DR, SP1, DR and a poly (T)₇ terminator. gRNA cassette 3 comprised the synthetic Pol III promoter GSP2239 (SEQ ID NO: 12) operably linked to a transcribable sequence comprising, in order: a DR (SEQ ID NO:9), spacer SP1 (SEQ ID NO:7), a DR, SP1, DR and a poly (T)₇ terminator. gRNA cassette 4 comprised a synthetic Pol III promoter GSP2244 (SEQ ID NO: 13) operably linked to a transcribable sequence comprising, in order: a DR (SEQ ID NO:9), spacer SP1 (SEQ ID NO:7), a DR, SP1, DR and a poly (T)₇ terminator. For each gRNA transcript, the DR-SP1-DR-SP1 portion of the transcript is a pre-crRNA precursor RNA that can be processed by Cas12a into two copies of mature SP1 guide RNAs.

Seven Cas12a and gRNA-expressing vectors were designed to generate targeted mutations within the Bmr3 gene (see Table 2). Each vector had a functional cassette for the expression of Cas12a (also known as Cpf1). pM223 was identical to pM325 described above except that the spacer sequence SP1 within the gRNA cassette was replaced by SP2 (SEQ ID NO: 8). pM225 was identical to pM327 described above except that all the spacer SP1 sequences within the gRNA cassette were replaced with SP2 (SEQ ID NO: 8). pM226 was identical to pM328 described above except that all spacer SP1 sequences within the gRNA cassette were replaced with SP2 (SEQ ID NO: 8). pM226 was identical to pM328 described above except that all spacer SP1 sequences within the gRNA cassette were replaced with SP2 (SEQ ID NO: 8). pM229 was identical to pM329 described above except that all spacer SP1 sequences within the two gRNA cassettes were replaced with SP2 (SEQ ID NO: 8). pM230 was identical to pM330 described above except that all spacer SP1 sequences within the two gRNA cassettes were replaced with SP2 (SEQ ID NO: 8). pM200 was identical to pM331 described above except that all spacer SP1 sequences within the four gRNA cassettes were replaced with SP2 (SEQ ID NO: 8). pM199 was identical to pM332 except that all spacer SP1 sequences within the four gRNA cassettes were replaced with SP2 (SEQ ID NO: 8). All vectors also comprised an expression cassette for the expression of a selectable marker (CP4) conferring resistance to the herbicide glyphosate.

TABLE 2
Vectors and gRNA cassette targeting GA20 oxidase_3 and Bmr3 genes.

| | | | | |
|---|---|---|---|---|
| Design | | | | Cassette elements |
| (Promoter | | # of gRNA | Cassette elements | SEQ ID NOs |
| X Repeat) | Vector | cassettes | (Prom::gRNA::polyT) | (Prom::gRNA) |

GA20 oxidase_3 targeting vectors

| | | | | |
|---|---|---|---|---|
| 1PX1 | pM325 | gRNA | GSP2262::DR-SP1-DR | SEQ ID NO.: 10:SEQ ID |
| | | cassette 1 | | NO.: 9-SEQ ID NO.: 7-SEQ |
| | | | | ID NO.: 9 |
| 1PX4 | pM327 | gRNA | GSP2262::DR-SP1-DR- | SEQ ID NO: 10::SEQ ID |
| | | cassette 1 | SP1-DR-SP1-DR-SP1- | NO: 9-SEQ ID NO: 7-SEQ ID |
| | | | DR | NO 9-SEQ ID NO: 7-SEQ ID |
| | | | | NO 9-SEQ ID NO: 7-SEQ ID |
| | | | | NO 9-SEQ ID NO: 7-SEQ ID |
| | | | | NO 9 |
| 1PX8 | pM328 | gRNA | GSP2262::DR-SP1-DR- | SEQ ID NO: 10::SEQ ID |
| | | cassette 1 | SP1-DR-SP1-DR-SP1- | NO: 9-SEQ ID NO: 7-SEQ ID |
| | | | DR-SP1-DR-SP1-DR- | NO 9-SEQ ID NO: 7-SEQ ID |
| | | | SP1-DR-SP1-DR | NO 9-SEQ ID NO: 7-SEQ ID |
| | | | | NO 9-SEQ ID NO: 7-SEQ ID |
| | | | | NO 9-SEQ ID NO: 7-SEQ ID |
| | | | | NO 9-SEQ ID NO: 7-SEQ ID |
| | | | | NO 9-SEQ ID NO: 7-SEQ ID |
| | | | | NO 9-SEQ ID NO: 7-SEQ ID |
| | | | | NO 9 |
| 2PX1 | pM329 | gRNA | GSP2262::DR-SP1-DR | SEQ ID NO: 10::SEQ ID |
| | | cassette 1 | | NO: 9-SEQ ID NO: 7-SEQ ID |
| | | | | NO 9 |
| | | gRNA | GSP2273::DR-SP1-DR | SEQ ID NO: 11::SEQ ID |
| | | cassette 2 | | NO: 9-SEQ ID NO: 7-SEQ ID |
| | | | | NO 9 |
| 2PX4 | pM330 | gRNA | GSP2262::DR-SP1-DR- | SEQ ID NO: 10::SEQ ID |
| | | cassette 1 | SP1-DR-SP1-DR-SP1- | NO: 9-SEQ ID NO: 7-SEQ ID |
| | | | DR | NO 9-SEQ ID NO: 7-SEQ ID |
| | | | | NO 9-SEQ ID NO: 7-SEQ ID |
| | | | | NO 9-SEQ ID NO: 7-SEQ ID |
| | | | | NO 9 |
| | | gRNA | GSP2273::DR-SP1-DR- | SEQ ID NO: 11::SEQ ID |
| | | cassette 2 | SP1-DR-SP1-DR-SP1- | NO: 9-SEQ ID NO: 7-SEQ ID |
| | | | DR | NO 9-SEQ ID NO: 7-SEQ ID |
| | | | | NO 9-SEQ ID NO: 7-SEQ ID |
| | | | | NO 9-SEQ ID NO: 7-SEQ ID |
| | | | | NO 9 |
| 4PX1 | pM331 | gRNA | GSP2262::DR-SP1-DR | SEQ ID NO: 10::SEQ ID |
| | | cassette 1 | | NO: 9- SEQ ID NO: 7-SEQ ID |
| | | | | NO 9 |
| | | gRNA | GSP2273::DR-SP1-DR | SEQ ID NO: 11::SEQ ID |
| | | cassette 2 | | NO: 9- SEQ ID NO: 7-SEQ ID |
| | | | | NO 9 |
| | | gRNA | GSP2239::DR-SP1-DR | SEQ ID NO: 12::SEQ ID |
| | | cassette 3 | | NO: 9- SEQ ID NO: 7-SEQ ID |
| | | | | NO 9 |
| | | gRNA | GSP2244::DR-SP1-DR | SEQ ID NO: 13::SEQ ID |
| | | cassette 4 | | NO: 9- SEQ ID NO: 7-SEQ ID |
| | | | | NO 9 |
| 4PX2 | pM332 | gRNA | GSP2262::DR-SP1-DR- | SEQ ID NO: 10::SEQ ID |
| | | cassette 1 | SP1-DR | NO: 9-SEQ ID NO: 7-SEQ ID |
| | | | | NO 9-SEQ ID NO: 7-SEQ ID |
| | | | | NO 9 |
| | | gRNA | GSP2273::DR-SP1-DR- | SEQ ID NO: 11::SEQ ID |
| | | cassette 2 | SP1-DR | NO: 9-SEQ ID NO: 7-SEQ ID |
| | | | | NO 9-SEQ ID NO: 7-SEQ ID |
| | | | | NO 9 |
| | | gRNA | GSP2239::DR-SP1-DR- | SEQ ID NO: 12::SEQ ID |
| | | cassette 3 | SP1-DR | NO: 9-SEQ ID NO: 7-SEQ ID |
| | | | | NO 9-SEQ ID NO: 7-SEQ ID |
| | | | | NO 9 |
| | | gRNA | GSP2244::DR-SP1-DR- | SEQ ID NO: 13::SEQ ID |
| | | cassette 4 | SP1-DR | NO: 9-SEQ ID NO: 7-SEQ ID |
| | | | | NO 9-SEQ ID NO: 7-SEQ ID |
| | | | | NO 9 |

Bmr3 targeting vectors

| | | | | |
|---|---|---|---|---|
| 1PX1 | pM223 | gRNA | GSP2262::DR-SP2-DR | SEQ ID NO:10::SEQ ID |
| | | cassette 1 | | NO: 9-SEQ ID NO: 8-SEQ ID |
| | | | | NO: 9 |
| 1PX4 | pM225 | gRNA | GSP2262::DR-SP2-DR- | SEQ ID NO: 10::SEQ ID |
| | | cassette 1 | SP2-DR-SP2-DR-SP2- | NO: 9-SEQ ID NO: 8-SEQ ID |
| | | | DR | NO 9-SEQ ID NO: 8-SEQ ID |
| | | | | NO 9-SEQ ID NO: 8-SEQ ID |
| | | | | NO 9-SEQ ID NO: 8-SEQ ID |
| | | | | NO 9 |
| 1PX8 | pM226 | gRNA | GSP2262::DR-SP2-DR- | SEQ ID NO: 10::SEQ ID |
| | | cassette 1 | SP2-DR-SP2-DR-SP2- | NO:9- SEQ ID NO: 8- SEQ ID |
| | | | DR-SP2-DR-SP2-DR- | NO 9- SEQ ID NO: 8- SEQ ID |
| | | | SP2-DR-SP2-DR | NO 9- SEQ ID NO: 8- SEQ ID |
| | | | | NO 9- SEQ ID NO: 8- SEQ ID |
| | | | | NO 9- SEQ ID NO: 8- SEQ ID |
| | | | | NO 9- SEQ ID NO: 8- SEQ ID |
| | | | | NO 9- SEQ ID NO: 8- SEQ ID |
| | | | | NO 9- SEQ ID NO: 8- SEQ ID |
| | | | | NO 9 |
| 2PX1 | pM229 | gRNA | GSP2262::DR-SP2-DR | SEQ ID NO: 10::SEQ ID |
| | | cassette 1 | | NO: 9-SEQ ID NO: 8-SEQ ID |
| | | | | NO 9 |
| | | gRNA | GSP2273::DR-SP2-DR | SEQ ID NO: 11::SEQ ID |
| | | cassette 2 | | NO: 9-SEQ ID NO: 8-SEQ ID |
| | | | | NO 9 |
| 2PX4 | pM230 | gRNA | GSP2262::DR-SP2-DR- | SEQ ID NO: 10::SEQ ID |
| | | cassette 1 | SP2-DR-SP2-DR-SP2- | NO: 9-SEQ ID NO: 8-SEQ ID |
| | | | DR | NO 9-SEQ ID NO: 8-SEQ ID |
| | | | | NO 9-SEQ ID NO: 8-SEQ ID |
| | | | | NO 9-SEQ ID NO: 8-SEQ ID |
| | | | | NO 9 |
| | | gRNA | GSP2273::DR-SP2-DR- | SEQ ID NO: 11::SEQ ID |
| | | cassette 2 | SP2-DR-SP2-DR-SP2- | NO: 9-SEQ ID NO: 8-SEQ ID |
| | | | DR | NO 9-SEQ ID NO: 8-SEQ ID |
| | | | | NO 9-SEQ ID NO: 8-SEQ ID |
| | | | | NO 9-SEQ ID NO: 8-SEQ ID |
| | | | | NO 9 |
| 4PX1 | pM200 | gRNA | GSP2262::DR-SP2-DR | SEQ ID NO: 10::SEQ ID |
| | | cassette 1 | | NO: 9-SEQ ID NO: 8-SEQ ID |
| | | | | NO 9 |
| | | gRNA | GSP2273::DR-SP2-DR | SEQ ID NO: 11::SEQ ID |
| | | cassette 2 | | NO: 9-SEQ ID NO: 8-SEQ ID |
| | | | | NO 9 |
| | | gRNA | GSP2239::DR-SP2-DR | SEQ ID NO: 12::SEQ ID |
| | | cassette 3 | | NO: 9-SEQ ID NO: 8-SEQ ID |
| | | | | NO 9 |
| | | gRNA | GSP2244::DR-SP2-DR | SEQ ID NO: 13::SEQ ID |
| | | cassette 4 | | NO: 9-SEQ ID NO: 8-SEQ ID |
| | | | | NO 9 |
| 4PX2 | pM199 | gRNA | GSP2262::DR-SP2-DR- | SEQ ID NO: 10::SEQ ID |
| | | cassette 1 | SP2-DR | NO: 9-SEQ ID NO: 8-SEQ ID |
| | | | | NO 9-SEQ ID NO: 8-SEQ ID |
| | | | | NO 9 |
| | | gRNA | GSP2273::DR-SP2-DR- | SEQ ID NO: 11::SEQ ID |
| | | cassette 2 | SP2-DR | NO: 9-SEQ ID NO: 8-SEQ ID |
| | | | | NO 9-SEQ ID NO: 8-SEQ ID |
| | | | | NO 9 |
| | | gRNA | GSP2239::DR-SP2-DR- | SEQ ID NO: 12::SEQ ID |
| | | cassette 3 | SP2-DR | NO: 9-SEQ ID NO: 8-SEQ ID |
| | | | | NO 9-SEQ ID NO: 8-SEQ ID |
| | | | | NO 9 |
| | | gRNA | GSP2244::DR-SP2-DR- | SEQ ID NO: 13::SEQ ID |
| | | cassette 4 | SP2-DR | NO: 9-SEQ ID NO: 8-SEQ ID |
| | | | | NO 9-SEQ ID NO: 8-SEQ ID |
| | | | | NO 9 |

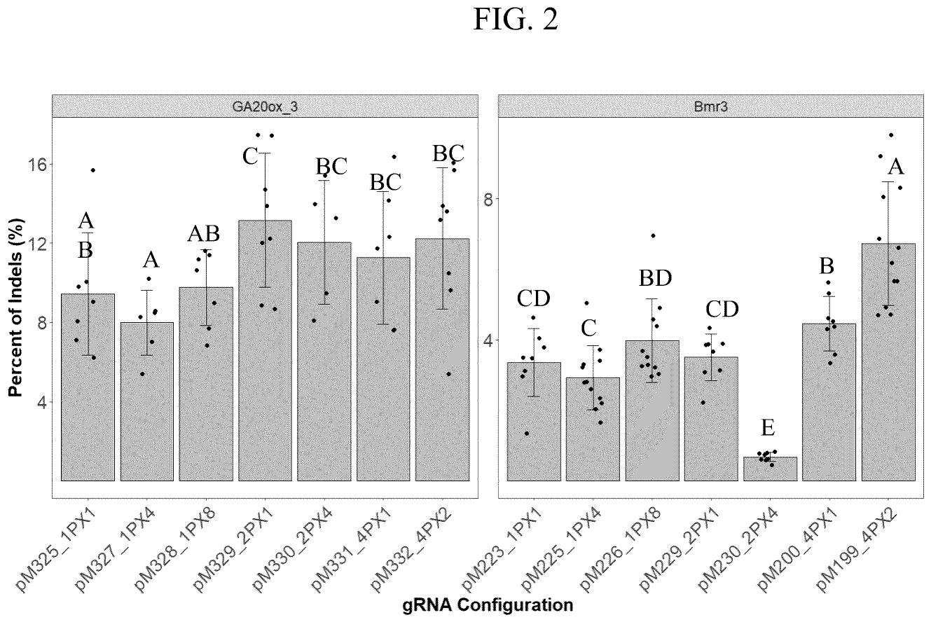

Corn leaf protoplasts were transfected using a PEG-based transfection method, similar to those known in the art, with the vectors described in Table 2. A control transfection was performed using pM098 that lacked the Cas12a and gRNA cassettes. Genomic DNA was isolated from the protoplast cells after transfection and incubation and target regions were amplified by PCR. The amplicons were sequenced by Next Generation Sequencing (NGS), using standard methods known in the art to identify modified sequences comprising insertions or deletions (InDels) around the GA20 oxidase or Bmr3 target sites that are indicative of editing. Each test transfection was repeated (rep) multiple times and a InDel % rates were calculated based upon the number of reps using Welch's t-test Table 3 and FIG. 2 show the InDel rate for each assay.

TABLE 3
Protoplast editing rates at the GA20
oxidase_3 and Bmr3 target sites.

| Design | | | InDel Rate (%) | |
|---|---|---|---|---|
| (Promoter X | | | | Statistical |
| Repeat) | Vector | N | Mean ± SE | Analysis* |
| Control | p098 | 14 | 0.02 ± 0.01 | — |

GA20 oxidase_3 targeting vectors with high efficiency guide RNA

| | | | | |
|---|---|---|---|---|
| 1PX1 | pM325 | 7 | 9.43 ± 1.17 | AB |
| 1PX4 | pM327 | 6 | 7.99 ± 0.67 | A |
| 1PX8 | pM328 | 7 | 9.77 ± 0.73 | AB |
| 2PX1 | pM329 | 8 | 13.16 ± 1.20 | C |
| 2PX4 | pM330 | 5 | 12.04 ± 1.39 | BC |
| 4PX1 | pM331 | 7 | 11.27 ± 1.27 | BC |
| 4PX2 | pM332 | 8 | 12.24 ± 1.26 | BC |

Bmr3 targeting vectors with moderate or low efficiency guide RNA

| | | | | |
|---|---|---|---|---|
| 1PX1 | pM223 | 8 | 3.36 ± 0.34 | CD |
| 1PX4 | pM225 | 12 | 2.93 ± 0.26 | C |
| 1PX8 | pM226 | 11 | 3.98 ± 0.36 | BD |
| 2PX1 | pM229 | 8 | 3.51 ± 0.24 | CD |
| 2PX4 | pM230 | 8 | 0.68 ± 0.05 | E |
| 4PX1 | pM200 | 8 | 4.46 ± 0.27 | B |
| 4PX2 | pM199 | 12 | 6.73 ± 0.51 | A |

N equals number of reps SE represents Standard Error.
*The letters represent significantly different means.
If two rows have the same letters, they are not significantly different.
If the letters are different, then the means are significantly different.

The data shown in the Table 3 and FIG. 2 suggest that it is more effective to increase gRNA expression through multiple gRNA cassettes driven by different promoters than through adding spacer array repeats driven by a single cassette or promoter. For example, the mean editing rates for 4PX1, 4PX2 configurations trended higher than the 1PX4 configuration for both target sites, with difference among Bmr3 means being statistically significant. Repeating gRNA cassettes (i.e.: gRNA titration) thus has a stronger effect on editing rates for the gRNA with moderate editing efficiency (e.g.: SP2 targeting Bmr3).

### Example 2

Assay for Optimizing Expression of a gRNA that Targets the Bmr3 Genomic Locus Using Transfected Protoplasts

This protoplast experiment was designed to test the effect of increasing guide RNA expression using different promoters while keeping Cas12a levels constant. Four unique Pol III promoters were used to drive the expression of a gRNA in a simplex or additive fashion.

Seven Cas12a and gRNA-expressing vectors were designed to generate targeted mutations within the Bmr3 gene. Each vector had a functional cassette for the expression of Cas12a (or Cpf1). The Cas12a expression cassette comprised a constitutive maize Ubiquitin promoter P-Zm.UbqM1 (SEQ ID NO: 14), operably linked 5′ to a plant codon optimized sequence for Lachnospiraceae bacterium Cas12a RNA-guided endonuclease (SEQ ID NO: 2). The protein sequence of LbCas12a is set forth as SEQ ID NO:4. The LbCas12a DNA sequence was flanked by DNA sequences encoding nuclear localization signals (NLS) at the 5′ and 3′ ends (SEQ ID: 52 and SEQ ID: 53) and operably linked 5′ to a transcription terminator sequence from a rice Lipid transfer protein (LTP) gene (SEQ ID NO:3).

TABLE 4
Vectors and gRNA cassette targeting the Bmr3 genes.

| Design | | | | Cassette elements |
|---|---|---|---|---|
| (Promoter | | # of gRNA | Cassette elements | SEQ ID NOs |
| X Repeat) | Vector | cassettes | (Prom::gRNA::polyT) | (Prom::gRNA) |
| 1PX1 | pM431 | gRNA | GSP2239::DR-SP2 | SEQ ID NO: 12::SEQ ID |
| | | cassette 1 | | NO: 9-SEQ ID NO: 8 |
| 1PX1 | pM432 | gRNA | GSP2244::DR-SP2 | SEQ ID NO: 13::SEQ ID |
| | | cassette 1 | | NO: 9-SEQ ID NO: 8- |
| | | | | SEQ ID NO 9 |
| 1PX1 | pM433 | gRNA | GSP2233::DR-SP2 | SEQ ID NO: 15::SEQ ID |
| | | cassette 1 | | NO: 9-SEQ ID NO: 8 |
| 1PX1 | pM434 | gRNA | GSP2245::DR-SP2 | SEQ ID NO: 16::SEQ ID |
| | | cassette 1 | | NO: 9-SEQ ID NO: 8 |
| 2PX1 | pM677 | gRNA | GSP2244::DR-SP2-DR- | SEQ ID NO: 13::SEQ ID |
| | | cassette 1 | SP2-DR-SP2-DR-SP2 | NO: 9-SEQ ID NO: 8 |
| | | gRNA | GSP2239::DR-SP2-DR- | SEQ ID NO: 12::SEQ ID |
| | | cassette 2 | SP2-DR-SP2-DR-SP2 | NO: 9-SEQ ID NO: 8 |
| 3PX1 | pM678 | gRNA | GSP2233::DR-SP2 | SEQ ID NO: 15::SEQ ID |
| | | cassette 1 | | NO: 9-SEQ ID NO: 8 |
| | | gRNA | GSP2244::DR-SP2 | SEQ ID NO: 13::SEQ ID |
| | | cassette 2 | | NO: 9-SEQ ID NO: 8 |
| | | gRNA | GSP2239::DR-SP2 | SEQ ID NO: 12::SEQ ID |
| | | cassette 3 | | NO: 9-SEQ ID NO: 8 |
| 4PX1 | pM679 | gRNA | GSP2245::DR-SP2 | SEQ ID NO: 16::SEQ ID |
| | | cassette 1 | | NO: 9-SEQ ID NO: 8 |
| | | gRNA | GSP2233::DR-SP2 | SEQ ID NO: 15::SEQ ID |
| | | cassette 2 | | NO: 9-SEQ ID NO: 8 |
| | | gRNA | GSP2244::DR-SP2 | SEQ ID NO: 13::SEQ ID |
| | | cassette 3 | | NO: 9-SEQ ID NO: 8 |
| | | gRNA | GSP2239::DR-SP2 | SEQ ID NO: 12::SEQ ID |
| | | cassette 4 | | NO: 9-SEQ ID NO: 8 |

As shown in Table 4, pM431 comprised a single guide RNA cassette comprising a single copy of the spacer SP2. The cassette comprised a synthetic RNA Polymerase III (Pol III) promoter GSP2239 (SEQ ID NO: 12) operably linked to a transcribable sequence comprising, in order: a Cas12a-compatible Direct repeat (DR) sequence (SEQ ID NO:9) and spacer SP2 (SEQ ID NO:8) and a poly (T)₇ terminator. pM432 comprised a single guide RNA cassette comprising a single copy of the spacer SP2. The cassette comprised a synthetic Pol III promoter GSP2244 (SEQ ID NO: 13) operably linked to a transcribable sequence comprising, in order: a DR sequence (SEQ ID NO: 9), spacer SP2 (SEQ ID NO:8) and a poly (T)₇ terminator. pM433 comprised a single guide RNA cassette comprising a single copy of the spacer SP2. The cassette comprised a synthetic Pol III promoter GSP2233 (SEQ ID NO: 15) operably linked to a transcribable sequence comprising, in order: a DR sequence (SEQ ID NO:9), spacer SP2 (SEQ ID NO:8), and a poly (T)₇ terminator. pM434 comprised a single guide RNA cassette comprising a single copy of the spacer SP2. The cassette comprised a synthetic Pol III promoter GSP2245 (SEQ ID NO: 16) operably linked to a transcribable sequence comprising, in order: a DR sequence (SEQ ID NO:9), spacer SP2 (SEQ ID NO: 8), and a poly (T)₇ terminator.

pM677 comprised two guide RNA cassettes each driven by a unique Pol III promoter and comprising a single copy of the spacer SP2. gRNA cassette 1 comprised a synthetic Pol III promoter GSP2244 (SEQ ID NO: 13) operably linked to a transcribable sequence comprising, in order: a Cas12a-compatible Direct repeat (DR) (SEQ ID NO:9), spacer SP2 (SEQ ID NO:8), and DR. gRNA cassette 2 comprised a synthetic Pol III promoter GSP2239 (SEQ ID NO: 12) operably linked to a transcribable sequence comprising, in order: a DR sequence (SEQ ID NO:9), spacer SP2 (SEQ ID NO:9) and a poly (T)₇ terminator.

pM678 comprised three guide RNA cassettes each driven by a unique Pol III promoter and comprising a single copy of the spacer SP2. gRNA cassette 1 comprised a synthetic Pol III promoter GSP2233 (SEQ ID NO: 15) operably linked to a transcribable sequence comprising, in order: a Cas12a-compatible Direct repeat (DR) (SEQ ID NO:9), a spacer SP2 (SEQ ID NO:8), and a poly (T)₇ terminator. gRNA cassette 2 comprised a synthetic Pol III promoter GSP2244 (SEQ ID NO: 13) operably linked to a transcribable sequence comprising, in order: a DR (SEQ ID NO:9), spacer SP2 (SEQ ID NO:8) and a poly (T)₇ terminator. gRNA cassette 3 comprised a synthetic Pol III promoter GSP2239 (SEQ ID NO: 12) operably linked to a transcribable sequence comprising, in order: a DR sequence (SEQ ID NO:9), spacer SP2 (SEQ ID NO:9), and a poly (T)₇ terminator.

pM679 comprised four guide RNA cassettes each driven by a unique Pol III promoter and comprising a single copy of the spacer SP2. gRNA cassette 1 comprised a synthetic Pol III promoter GSP2245 (SEQ ID NO: 16) operably linked to a transcribable sequence comprising, in order: a Cas12a-compatible Direct repeat (DR) (SEQ ID NO:9), a spacer SP2 (SEQ ID NO:8), and a poly (T)₇ terminator. gRNA cassette 2 comprised a synthetic Pol III promoter GSP2233 (SEQ ID NO: 15) operably linked to a transcribable sequence comprising, in order: a DR (SEQ ID NO:9), spacer SP2 (SEQ ID NO:8), and a poly (T)₇ terminator. gRNA cassette 3 comprised a synthetic Pol III promoter GSP2244 (SEQ ID NO: 13) operably linked to a transcribable sequence comprising, in order: a DR sequence (SEQ ID NO:9), spacer SP2 (SEQ ID NO:8) and a poly (T)₇ terminator. gRNA cassette 4 comprised a synthetic Pol III promoter GSP2239 (SEQ ID NO: 12) operably linked to a transcribable sequence comprising, in order: a DR (SEQ ID NO:9), spacer SP2 (SEQ ID NO:9), and a poly (T)₇ terminator. All vectors also comprised an expression cassette for the expression of a selectable marker conferring resistance to the herbicide glyphosate.

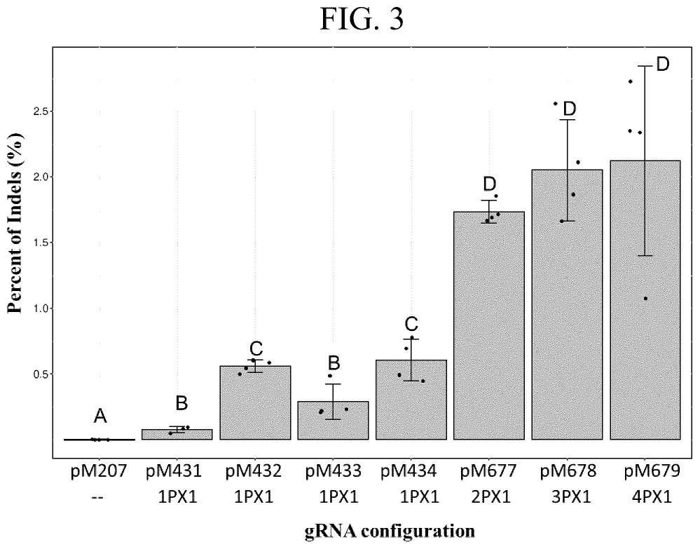

Corn leaf protoplasts were transfected using a PEG-based transfection method, similar to those known in the art, with the vectors described in Table 4. A control transfection was performed using the vector pM207 that lacked any gRNA cassettes. Genomic DNA was isolated from the protoplast cells after transfection and incubation and Bmr3 target regions were amplified by PCR. The amplicons were sequenced by Next Generation Sequencing (NGS), using standard methods known in the art to identify modified sequences comprising insertions or deletions (InDels) around the Bmr3 target site that are indicative of guide RNA-Cas12a mediated editing. Each test transfection was repeated four times and InDel rate (%) with accompanying 95% estimate uncertainity intervals were calculated based upon the number of replications using Welch's t-test. Table 5 and FIG. 3 show the InDel rate for each assay.

TABLE 5
Protoplast editing rates at the Bmr3 target site when four synthetic
Pol III promoters were tested in simplex and additive fashion.

| | | InDel Rate (%) | |
|---|---|---|---|
| Assay | | | Statistical |
| Design | Vector | Mean ± SE | Analysis* |
| Control | pM207(control) | 0.002 ± 0.003 | A |
| 1PX1 | pM431 | 0.077 ± 0.029 | B |
| 1PX1 | pM432 | 0.561 ± 0.046 | C |
| 1PX1 | pM433 | 0.288 ± 0.133 | B |
| 1PX1 | pM434 | 0.605 ± 0.154 | C |
| 2PX1 | pM677 | 1.734 ± 0.083 | D |
| 3PX1 | pM678 | 2.051 ± 0.378 | D |
| 4PX1 | pM679 | 2.124 ± 0.707 | D |

SE represents Standard Error.
*If two rows have the same letters, they are not significantly different.
If the letters are different, then the means are significantly different

As can be seen in Table 5 and FIG. 3, adding several single gRNA cassettes, under the control different promoters, targeting a single genomic site to a vector comprising a single Cas12a cassette, exhibited strong correlation with increased chromosome cutting. Using this method, more than 20-fold-improvement of chromosome cutting between a singleton (1PX1, pM431) and a quadruplex (4PX1, pM679) gRNA cassette was observed within the plant protoplast system.

### Example 3

In planta assay for optimizing expression of a gRNA that targets the Bmr3 genomic locus

This in planta experiment was designed to test the effect of increasing guide RNA expression using different promoters while keeping Cas12a levels constant. Five unique Pol III promoters were used to drive the expression of the Bmr3 gRNA in a simplex or additive fashion.

Seven Cas12a and gRNA-expressing vectors, described in Table 4 and Example 2, were used to test the in planta expression and editing efficiencies of each vector. An eighth vector, pM435, was also designed. pM435 was identical to pM431 described in Example 2, except that instead of a synthetic Pol III promoter, the gRNA expression was driven by a chimeric Pol III promoter (SEQ ID NO:8). Corn embryos were transformed with the vectors described above by Agrobacterium-mediated transformation and plants were regenerated from the transformed corn cells. DNA was extracted from leaf samples from 84 regenerated seedlings generated from each vector. Bmr3 target regions were amplified by PCR. The amplicons were sequenced by Next Generation Sequencing (NGS), using standard methods known in the art to identify modified sequences comprising insertions or deletions (InDels) around the Bmr3 target site that are indicative of guide RNA-Cas12a mediated editing (see Yang et.al., A next-generation marker genotyping platform (AmpSeq) in heterozygous crops: a case study for marker-assisted selection in grapevine. Hortic. Res. 3, 16002,2016). A Taqman based assay was performed to identify the copy number of the Cas12a cassette in each plant. For comparative analysis across populations, plants carrying only one or two copies of the Cas12a cassette were analysed for edits within the target site. A plant was called edited at an ‘advanceable’ level if at least ten percent of its sequence reads covering the target site carried InDels (Table 6). A similar comparative analyis was carried out for RO plants comprising only single copy events (Table 7).

TABLE 6
In planta editing rates for one- and two- copy events
at the Bmr3 target site when four synthetic Pol III
promoters were tested in simplex and additive fashion.

| | | Plants assayed | Mean | | |
|---|---|---|---|---|---|
| Assay | | (1 and | InDel | Plants with advanceable | |
| Design | Vector | 2 copy events) | Rate (%) | editing rates | |
| 1PX1 | pM435 | 38 | 2.82 | 1 | (2.63%) |
| 1PX1 | pM431 | 34 | 2.26 | 2 | (5.88%) |
| 1PX1 | pM432 | 41 | 4.19 | 3 | (7.32%) |
| 1PX1 | pM433 | 27 | 7.65 | 3 | (11.11%) |
| 1PX1 | pM434 | 33 | 8.15 | 4 | (12.12%) |
| 2PX1 | pM677 | 35 | 8.09 | 5 | (14.29%) |
| 3PX1 | pM678 | 50 | 11.47 | 10 | (20.00%) |
| 4PX1 | pM679 | 34 | 17.48 | 7 | (20.59%) |

TABLE 7
In planta editing rates for one-copy events at the
Bmr3 target site when four synthetic Pol III promoters
were tested in simplex and additive fashion.

| | | | Mean | | |
|---|---|---|---|---|---|
| Assay | | Plants assayed | InDel | Plants with advanceable | |
| Design | Vector | (1 copy events) | Rate (%) | editing rates | |
| 1PX1 | pM435 | 21 | 0.28 | 0 | (0.00%) |
| 1PX1 | pM431 | 23 | 1.54 | 1 | (4.35%) |
| 1PX1 | pM432 | 26 | 5.24 | 2 | (7.69%) |
| 1PX1 | pM433 | 23 | 3.15 | 2 | (8.70%) |
| 1PX1 | pM434 | 17 | 7.02 | 2 | (11.76%) |
| 2PX1 | pM677 | 18 | 4.38 | 1 | (5.56%) |
| 3PX1 | pM678 | 22 | 11.53 | 4 | (18.18%) |
| 4PX1 | pM679 | 14 | 21.63 | 3 | (21.43%) |

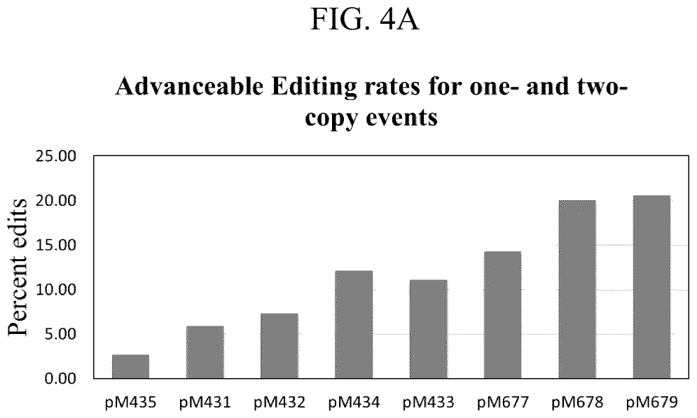

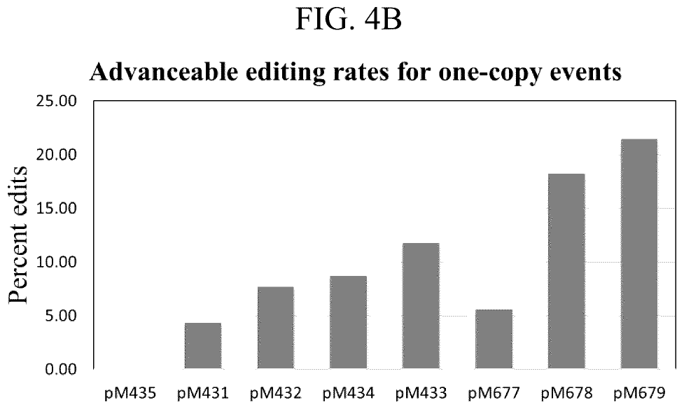

As can be seen in Tables 6-7 and FIG. 4, adding multiple gRNA cassettes targeting a single genomic site to a vector comprising a single Cas12a cassette exhibited a correlation with increased chromosome cutting. When compared to the protoplast experiment, the improvement was more moderate, and up to a 3.5-fold improvement in chromosome cutting between a singleton (1PX1) and a quadrupled (4PX1) gRNA cassette was observed. Neither the protoplast system nor the in planta data appeared to show signs of early silencing as judged by the increasing chromosome cutting rates with each additional gRNA cassette.

Taken together, the data from these experiments suggests that increasing gRNA expression for the CRISPR/Cas12a system is sufficient to elevate chromosome targeting significantly, even if expression of the Cas enzyme is expected to be unchanged. Without being bound by any scientific theory, the multiple gRNA cassettes may not trigger early gene silencing which may further contribute to improved editing. gRNA cassettes driven by Pol III promoters are significantly smaller than typical Cas nuclease cassettes. This implies that it is possible to increase chromosome cutting by tandem duplication of gRNA cassettes without the need to develop very large expression vectors, which can jeopardize clonal stability. Moreover, the designs described in these examples provide an excellent use case for diversified regulatory elements for gRNA expression. Using polymorphic/highly diversified regulatory elements as opposed to repeated use of the same element may also minimize clonal instability and gene silencing risk.

### Example 4

AsiSI/PacI based Modular assembly of gRNA cassettes.

This example describes a methodology for efficient, modular assembly of single or multiplex gRNA cassettes in a construct useful for transformation. The strategy relies on the use of the rare cutting restriction enzymes (RE) AsiSI (also known as SfaAl) and PacI with compatible cohesive ends. AsiSI recognizes and cleaves the unique 8 bp sequence 5′-GCGAT\hat{ }CGC-3′while PacI recognizes and cleaves the 8 bp sequence 5′-TTAATATAA-3′ so as to generate overhangs that are compatible with each other.

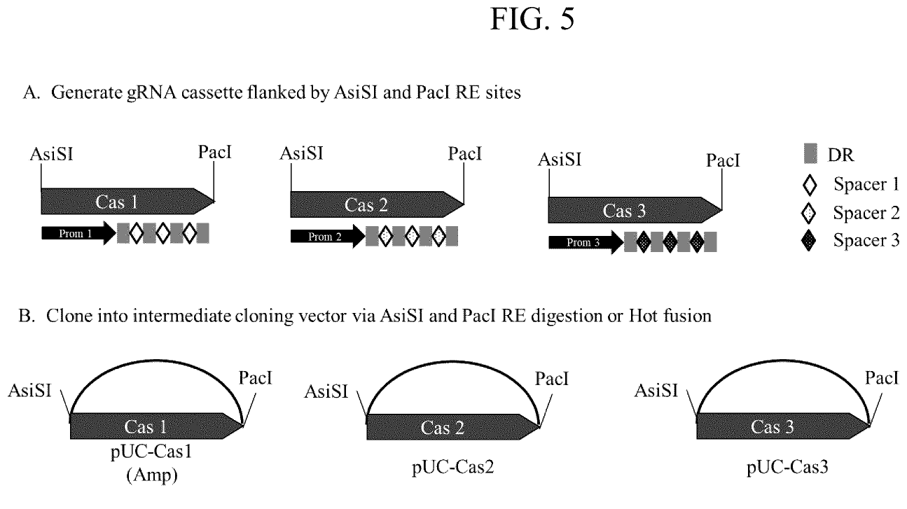

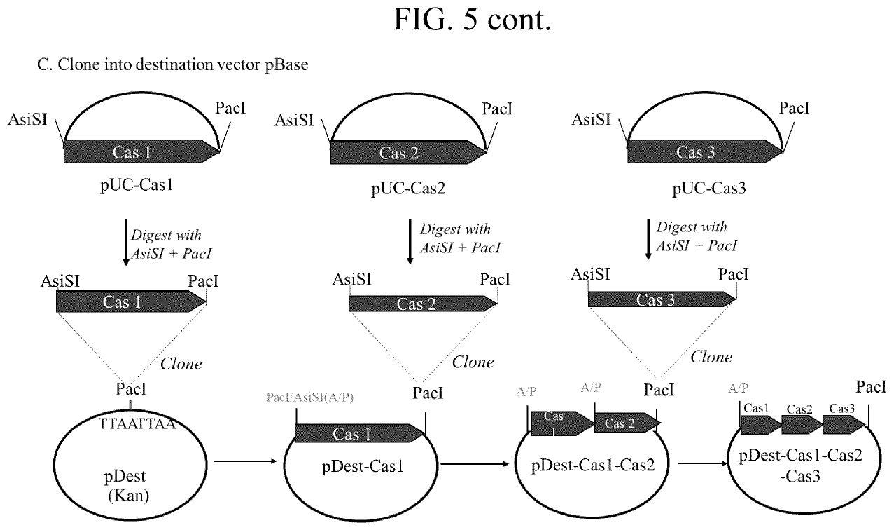

The steps and cloning strategy for generating a plant transformation vector comprising three gRNA cassettes (Cas1, Cas2, and Cas3) is outlined in FIG. 5. Each gRNA cassette comprises a promoter operably linked to a guide RNA comprising at least one Direct Repeat (DR), one spacer and a poly (T)₇ terminator. For exemplary purposes, each gRNA cassette in FIG. 5 is shown as comprising a promoter operably linked to a gRNA array comprising three spacers flanked by DRs in the following configuration DR-SP-DR-SP-DR-SP-DR. Each gRNA cassette is generated such that it is flanked by an AsiSI restriction enzyme site (5′-GCGAT\hat{ }CGC-3′) and a PacI (5′-TTAAT\hat{ }TAA-3′) restriction enzyme site (see FIG. 5, Panel A). The gRNA cassettes can be generated by synthesis or by PCR-based assembly using sequence specific primers comprising the restriction enzyme sites. Next, each cassette is cloned into an intermediate cloning vector comprising a suitable selection marker (eg: pUC, Amp+) that comprises a pair of AsiSI and PacI recognition sites so as to generate three single-cassette intermediary vectors: pUC-Cas1, pUC-Cas2 and pUC-Cas3 wherein each gRNA cassette is flanked by AsiSI and PacI restriction enzyme sites (see FIG. 5, Panel B). Next, a destination plant transformation vector (pDest) comprising a second selectable marker (eg. Kan+) and a unique PacI (5′-TTAAT TAA-3′) restriction enzyme site is created. In order to assemble the gRNA cassettes within the destination vector, pUC-Cas1 is digested with AsiSI and PacI so as to release Cas1 while pDest is linearized with the PacI enzyme. Gel purified DNA fragment indicated as cassette Cas1 is incubated with the linearized pDest vector in the presence of an appropriate ligase enzyme. Ligation of the Cas1 DNA fragment comprising a 5′ AsiSI overhang and a 3′ PacI overhang with pDest comprising a linearized PacI site, results in the generation of an AsiSI/PacI hybrid site (5′-TTAATCGC-3′) that is no longer recognized by either enzyme and reconstitutes a single PacI recognition site (5′-TTAATTAA-3′). Thus the resulting insertion of the Cas1 fragment into the PacI site within pDest generates the pDest-Cas1 vector and reconstitutes a new PacI site adjacent to the cassette Cas1 that is now available for the insertion of Cas2 (see FIG. 5, Panel C). In the FIG. 5, as an illustration, the reconstituted PacI site is located 3′ of the Cas1 cassette. However, the Cas1 cassette can also be inserted in inverted orientation, reconstituting a PacI site 5′ of the Cas1 cassette.

As a next step, pUC-Cas2 is digested with AsiSI and PacI to release the DNA fragment indicated as cassette Cas2 which is incubated with PacI linearized pDest-Cas1 and ligase resulting in the insertion of Cas2 adjacent to Cas1 (pDest-Cas1-Cas2) and the regeneration of a PacI site adjacent to cassette Cas2 and in the figure located 3′ to Cas2. The process is repeated one more time with AsiSI and PacI digested pUC-Cas3 and PacI linearized pDest-Cas1-Cas2 to ultimately generate pDest-Cas1-Cas2-Cas3. Since each insertion event results in the reconstitution of a PacI site adjacent to the inserted cassette, additional cassettes can be added adjacent to the existing stack. The process of adding cassettes to the stack could be repeated in unlimited fashion in theory but may be limited in practice by the size of the vector that can still be successfully used for transformation.

The above example and FIG. 5 illustrate the construction of multiple individual guide RNA expression cassettes (Cas) comprising four elements (promoter, Direct repeat, Spacer and a poly (T)₇ terminator) in a vector useful for transformation. As the cloning step of each individual guide RNA expression cassette is not directional, i.e. can be both in direct or inverted orientation, this will lead to various vectors with different combinations of the inserted cassettes. A person skilled in the art will however be able to use this method to assemble gene expression cassettes or gene silencing cassettes comprising other elements like Gene of Interest (GOI), enhancer, intron, 5′ leader, DNA elements encoding signaling peptides, miRNA, siRNA etc. A person skilled in the art will also realize that the restriction site in the destination vector could also be AsiSI instead of PacI. A person skilled in the art will also be able to use isoschizomers of AsiSI like the REs SfaAl, SgfI, and Rgal in lieu of AsiSI. A person skilled in the art will also be able to use this method with other 8-base pair rare cutting REs with compatible cohesive ends like AscI (also known as Sgs1) and MauB1. AscI recognizes and cleaves the unique 8 bp sequence 5′-GG CGCGCC-3′ while MauB1 recognizes and cleaves the 8 bp sequence 5′-CG CGCGCG-3′ so as to generate overhangs that are compatible with each other. In the latter case, the destination vector comprises a recognition site for AscI or MauB1.

### Example 5

gRNA Cassettes Driven by Diverse Pol III Promoters can be Stacked to Increase gRNA Expression and Editing Rates

This in planta experiment was designed to test multiplex gRNA configurations while keeping Cas12a expression levels constant. Six Cas12a vectors comprising Bmr3 gRNAs with moderate or low efficiency described in Example 1 were tested. To improve low editing rates, the same spacer was tested in various multiplex gRNA configurations. In one configuration, the spacer was repeated multiple times in the same gRNA array driven by one promoter. This was compared to a configuration in which the same spacer was placed in separate cassettes driven by diverse Pol III promoters. Seedlings were regenerated after transformation with each vector. Bmr3 target regions were amplified by PCR. The amplicons were sequenced by Next Generation Sequencing (NGS), using standard methods known in the art to identify modified sequences comprising insertions or deletions (InDels) around the Bmr3 target site that are indicative of guide RNA-Cas12a mediated editing (see Yang et.al., A next-generation marker genotyping platform (AmpSeq) in heterozygous crops: a case study for marker-assisted selection in grapevine. Hortic. Res. 3, 16002,2016). A Taqman-based assay was performed to identify the copy number of the Cas12a cassette in each plant. For comparative analysis across populations, plants carrying only one or two copies of the Cas12a cassette were analyzed for edits within the target site. A plant was called edited at an ‘advanceable’ level if at least ten percent of its sequence reads covering the target site carried InDels.

Additionally, RNA expression analysis was carried out. Leaf tissues were collected from regenerated seedlings at the VI-growth stage. Direct-zol RNA MiniPrep Kits (Zymo Research, Irvine, CA; www.zymoresearch.com) were used to extract total RNA according to manufacturer's instructions. A subset of RNA samples was run on 5300 Fragment Analyzer System (Agilent, Santa Clara, CA; www.agilent.com) to confirm RNA quality (RNA Integrity Number>7) . . .

First strand cDNAs were generated from processed, mature gRNAs using specific reverse transcription primers and Custom TaqMan Small RNA Assay kits (Applied Biosystems, Foster City, CA; www.thermofisher.com). The thermal cycling conditions for all assays were 98° C. for 10s followed by 40 cycles of 98° C. for Is, 60° C. for 5s, 72° C. for 30s using a CFX Real-Time Detection System (Bio-Rad, Hercules, CA; www.bio-rad.com). The Ct value for each transcript was determined by regression Bio-Rad CFX Manager software (Bio-Rad, Hercules, CA; www.bio-rad.com).

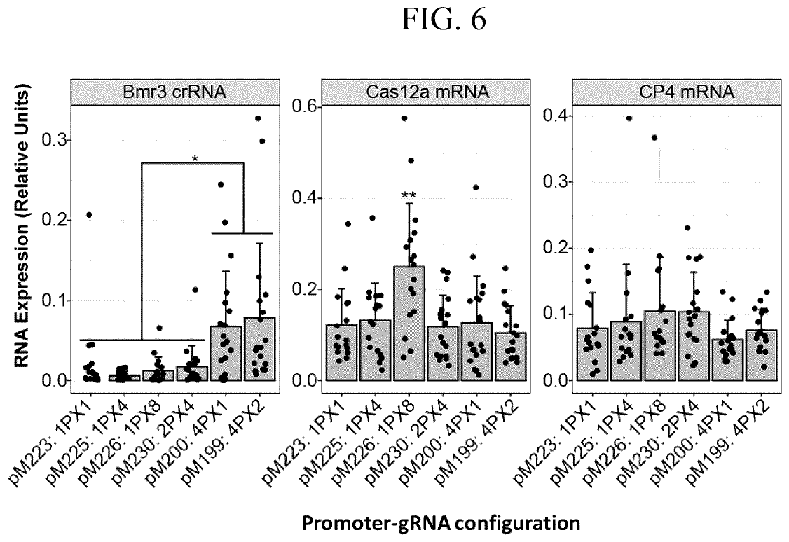

Multiple spacers in a single array did not improve editing rates in plants as compared to their singleton counterparts (Table 8). However, stacking multiple gRNA cassettes resulted in a significant increase in mature gRNA accumulation (FIG. 6). In contrast, selectable marker gene CP4 RNA expression levels, used as a control, were constant across constructs (FIG. 6). In addition, repeating the same spacer through multiple gRNA cassettes significantly increased the rate of advanceable edited plants from (Table 8). A construct with multiple spacers in a single array (pM226) did show significantly higher LbCas12a RNA expression but did not result in an increased editing rate or gRNA accumulation. Overall, adding multiple gRNA cassettes with different promoters improved expression and editing from a low efficiency gRNA. Intriguingly, a gRNA cassette in which a single promoter drove an array of the same spacer sequence in tandem did not produce the same results.

TABLE 8
In planta editing rates at the Bmr3 target site for various
gRNA configurations along with a single LbCas12a cassette.

| | | | Average indel | | |
|---|---|---|---|---|---|
| | | | rate in | Plants with | |
| | | Plants | sequencing | advanceable | |
| Assay design | Vector | assayed | reads | editing rates | |
| 1PX1 | pM223 | 56 | 0.2% | 0 | (0.0%) |
| 1PX4 | pM225 | 52 | 0.0% | 0 | (0.0%) |
| 1PX8 | pM226 | 59 | 0.0% | 0 | (0.0%) |
| 2PX4 | pM230 | 57 | 0.3% | 0 | (0.0%) |
| 4PX1 | pM200 | 48 | 5.9% | 5 | (10.4%) |
| 4PX2 | pM199 | 51 | 5.6% | 4 | (7.8%) |

1. A method for editing the genome of a plant cell at a target site in said cell comprising the steps of
providing an RNA guided effector protein, or a nucleic acid encoding said RNA guided effector protein, to said cell; and
providing a guide RNA recognizing said target site to said cell, wherein said guide RNA is expressed in said cell from at least two recombinant cassettes;
wherein each of the recombinant cassettes comprises a plant-expressible promoter operably linked to a nucleic acid encoding said guide RNA and wherein the promoter of each recombinant cassette is different from the plant-expressible promoter of the other recombinant cassette or cassettes expressing said guide RNA.

2. The method according to claim 1, wherein said RNA guided effector protein is a CRISPR-Cas effector protein, selected from a Type I CRISPR-Cas system, a Type II CRISPR-Cas system, a Type III CRISPR-Cas system, a Type IV CRISPR-Cas system, Type V CRISPR-Cas system, or a Type VI CRISPR-Cas system, or a CRISPR-Cas effector protein derived therefrom, optionally a CRISPR-Cas effector protein comprising one or more nuclear localization signals.

3. The method according to claim 1, wherein said RNA guided effector protein is a fusion protein comprising a cleavage domain, a nuclease domain, a deaminase domain, a cytosine deaminase domain, an adenine deaminase domain, a transcription activator domain, a transcription repression domain, a reverse transcriptase domain, a uracil DNA glycolase inhibitor, a Dna2 polypeptide, and/or a 5′ flap endonuclease.

4. The method according to claim 1, wherein said RNA guided effector protein comprises an amino acid sequence having at least 90% or 95% sequence identity to an amino acid sequence selected from any one of SEQ ID NOs: 4-6 or comprises a nucleotide sequence having at least 90% sequence identity to the nucleotide sequence of SEQ ID No: 2.

5. (canceled)

6. The method according to claim 1, wherein said promoter expressing said guide RNA(s) is/are DNA-dependent RNA polymerase III promoter(s) (Pol III promoter).

7. The method according to claim 6, wherein said Pol III promoter is selected from the group consisting of a U6 promoter, an H1 promoter, a 5S promoter, an Adenovirus 2 (Ad2) VAI promoter, a tRNA promoter, a 7SK promoter, chimeric Pol III promoters or synthetic Pol III promoters, wherein the Pol III promoter comprises a nucleotide sequence selected from any one of SEQ ID NOS: 10-13, 15-16 or 18-45.

8. (canceled)

9. (canceled)

10. The method according to claim 1, wherein said guide RNA is a guide RNA which results in moderate or poor editing or has a moderate or low editing efficiency at said target site, optionally a guide RNA which results in a cutting efficiency of less than 20%, or wherein said guide RNA has an editing efficiency score of less than 1, between 0 and 1 or less than 0.

11. (canceled)

12. A plant cell comprising at least two recombinant cassettes, each cassette expressing a guide RNA recognizing a target site in the genome of said cell under control of a plant-expressible promoter, wherein the guide RNA is the same or similar and recognizes the same target site, and wherein the plant-expressible promoter of each cassette is different from the plant-expressible promoter of the other recombinant cassette or cassettes, said plant cell optionally further comprising an RNA guided effector protein, or a nucleic acid encoding said RNA guided effector.

13. (canceled)

14. The plant cell according to claim 12, wherein said promoter(s) expressing said guide RNA(s) is/are (a) Pol III promoter(s).

15. The plant cell according to claim 14, wherein said Pol III promoters are selected from the group consisting of a U6 promoter, an H1 promoter, a 5S promoter, an Adenovirus 2 (Ad2) VAI promoter, a tRNA promoter, a 7SK promoter, chimeric Pol III promoters or synthetic Pol III promoters or wherein the Pol III promoters comprise a nucleotide sequence selected from any one of SEQ ID NOS: 10-13, 15-16 or 18-45.

16. The plant cell according to 12, wherein said guide RNA is a guide RNA which results in moderate or poor editing or has a moderate or low editing efficiency at said target site, optionally a guide RNA which results in a cutting efficiency of less than 20% or wherein said guide RNA has an editing efficiency score of less than 1, between 0 and 1 or less than 0.

17. (canceled)

18. The plant cell according to claim 12, wherein said plant cell is comprised within a plant.

19. A vector comprising at least two recombinant cassettes, or a set of at least two recombinant cassettes, each cassette expressing a guide RNA recognizing a target site in the genome of said cell under control of a promoter, wherein the guide RNA is the same or similar and recognizes the same target site, and wherein the promoter of each cassette is different from the promoter of the other recombinant cassette or cassettes.

20. The vector or set of at least two recombinant cassettes according to claim 19, wherein said promoter(s) expressing said guide RNA(s) is/are (a) Pol III promoter(s).

21. The vector or set of at least two recombinant cassettes according to claim 20, wherein said Pol III promoters are selected from the group consisting of a U6 promoter, an H1 promoter, a 5S promoter, an Adenovirus 2 (Ad2) VAI promoter, a tRNA promoter, a 7SK promoter, chimeric Pol III promoters or synthetic Pol III promoters, or wherein the Pol III promoters comprise a nucleotide sequence selected from any one of SEQ ID NOs: 10-13, 15-16 or 18-45.

22. (canceled)

23. The vector or set of at least two recombinant cassettes according to claim 19, wherein said guide RNA is a guide RNA which results in moderate or poor editing or has a moderate or low editing efficiency at said target site, optionally a guide RNA which results in a cutting efficiency of less than 20%.

24. The vector or the set of at least two recombinant cassettes according to claim 19, wherein said vector is comprised within a plant cell.

25. The vector or the set of at least two recombinant cassettes of claim 24, said plant cell further comprising an RNA guided effector protein, or a nucleic acid encoding said RNA guided effector protein.

26-30. (canceled)

31. A method for iterative modular cloning comprising the steps of providing a first DNA fragment comprising a recombinant cassette of interest flanked by two different restriction sites, each site recognized respectively by a different rare cutting endonuclease, whereby the rare cutting endonucleases recognize a restriction site comprising at least 8 nucleotides and whereby the rare cutting endonucleases generate compatible cohesive ends;
a. treating the first DNA fragment with both rare cutting endonucleases of step a;
b. providing a DNA destination vector comprising one restriction site recognized by only one of the rare cutting endonucleases of step a;
c. treating the destination vector with the one rare cutting endonuclease of step c and optionally dephosphorylating the treated destination vector;
d. mixing the first DNA fragment resulting from step b with the destination vector of step d;
e. optionally adding a DNA ligase;
f. obtaining a resulting destination vector wherein the DNA fragment is linked to the destination vector whereby the recognition site recognized by only one of the rare cutting endonucleases of step c is reconstituted in the resulting destination vector;
g. providing a second DNA fragment comprising a recombinant cassette of interest flanked by two different restriction sites, each site recognized respectively by a different rare cutting endonuclease of step a;
h. treating the second DNA fragment with both rare cutting endonucleases of step a;
i. treating the resulting destination vector of step g with the one rare cutting endonuclease of step c and optionally dephosphorylating the treated destination vector;
j. mixing the second DNA fragment resulting from step i with the resulting destination vector of step j;
k. optionally adding a DNA ligase;
l. obtaining a second resulting destination vector wherein the second DNA fragment is linked to the resulting destination vector, whereby the recognition site recognized by only one of the rare cutting endonucleases of step c is reconstituted in the second resulting destination vector;
m. optionally reiterating steps a to g with a further DNA fragment comprising a recombinant cassette of interest flanked by the two different restriction sites to obtain a further resulting destination vector.

32. The method according to claim 31 wherein one of the two different rare cutting endonuclease is PacI and the other rare cutting endonuclease is AsiSI, and wherein the recognition site in the destination vector is PacI or AsiSI, wherein one of the two different rare cutting endonuclease is Asc/and the other rare cutting endonuclease is MauB1, and wherein the recognition site in the destination vector is AscI or MauB1.

33. (canceled)


SEQUENCE LISTING (49 sequences)

SEQ ID NO: 1 - DNA, 1399 bp, Oryza sativa
  source 1..1399: mol_type = genomic DNA
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaag

SEQ ID NO: 2 - DNA, 3681 bp, synthetic construct
  source 1..3681: mol_type = other DNA
tcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 3 - DNA, 298 bp, Oryza sativa
  source 1..298: mol_type = genomic DNA
taatcgatcctccgatcccttaattaccataccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatg

SEQ ID NO: 4 - protein, 1228 aa, unidentified
  source 1..1228: note = Lachnospiraceae bacterium
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 5 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 6 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 7 - DNA, 23 bp, synthetic construct
  source 1..23: mol_type = other DNA
gacgaccctactgctactactac

SEQ ID NO: 8 - DNA, 23 bp, synthetic construct
  source 1..23: mol_type = other DNA
cggcagcgcgtcgtagcagttct

SEQ ID NO: 9 - DNA, 20 bp, unidentified
  source 1..20: mol_type = genomic DNA; note = Lachnospiraceae bacterium
aatttctactaagtgtagat

SEQ ID NO: 10 - DNA, 280 bp, synthetic construct
  source 1..280: mol_type = other DNA
aatctgtgaagagtttattcacgattaatctaggaggaagcgcgctttgttggctgtaggcccaaagcagcaatcaacagagtgtgtgagggccagctgctggaaggttcggttttttcaacaggcccacacgcgcgggctgggctgggctgggctggccacgttccttcggtagcagttcggtggagcaacgaggacggtggaatcgaagtttagtaccacctcggctgaagatacggagaaagaccagtttataacctgcctctggagcctgctgctc

SEQ ID NO: 11 - DNA, 282 bp, synthetic construct
  source 1..282: mol_type = other DNA
ttaccagctctgtgaaacttcacgaaacgcaatcggaaaacgaggtgggctgtgttacacagctgggcctgtgtcaacagcaacgaagcccacccaacagaggattgggcctcgatcaagcggcctcacaacgtgcggacacgcggtgcgaggccactctgtcagcagaagcccggctgggagcaacgagcaacttgtgcgctgcgctgaaggtttagtaccacatcggttgcgtaacgaccagcttaccaccttataagcttaaaccggagcgctgctttc

SEQ ID NO: 12 - DNA, 300 bp, synthetic construct
  source 1..300: mol_type = other DNA
ttagtaccagcaccagcacacggttttttggctacgcacaatcggatacgcaggcgaccagcccaattacaaggcaaaagaggacgtcaacaagcagtgcccacgtcgggcgtagaagcccaattgtcccgtgggccgtgggccggcccgtgcgagcagcccaccgcgggcgcactgcaattgcgcttatagcccagttcgcgcgcttgtgcggtgcgagagcgagaacagtttagtaccacctcgtccagtcacaaggacgcagaccactttataagcgcgctgctagctactgagatc

SEQ ID NO: 13 - DNA, 300 bp, synthetic construct
  source 1..300: mol_type = other DNA
tcgtaccaggcgccaaagagaagtgaaggttcttgagcaccgtgaagatttttttgtcaagcgaactcttcaaaaagagcaaaaaggcctacgagcccacttcctccgctctgatcgaagcccacttcgagaggccgggccgtaagcagcccaatacgagcccacgcgggctgcggggggcgcgaaaaggcgctgcgcttgggaggcaacgagcgcgggggcaacagtttgtttagtaccacctcggcaacccaacgcgagaaagaccaccttataacgcgcgcgcttgagcttgtagtc

SEQ ID NO: 14 - DNA, 2008 bp, Zea mays
  source 1..2008: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtc

SEQ ID NO: 15 - DNA, 300 bp, synthetic construct
  source 1..300: mol_type = other DNA
agcacttcttttaacaaggaaaaatcagaacttctgtgcaagtgttttgtcaagcttaaaagaatagagagaggctgaggcgagagagaatttggtcgcagaaaaacacaaaaagcccaactgggctgtggaagcccacggccgtgcgctcagttctaccgcgaagcccaatcggtgcgcttcctgcagcgctctagcgcgcttcggtggagcgcgcgcaatcgagcgagagtttagtaccacatcggccagcaacgcgaacgcagaccagtttataagccgcgctgctgcagcaccctc

SEQ ID NO: 16 - DNA, 300 bp, synthetic construct
  source 1..300: mol_type = other DNA
cgtgttttttatacaactcggagaagtgacctgcagttcgcacagttacttttagtaggagatagtacacaaaaaattctggcccacggaaagcagcaaggcgtgagggttggtcgtgggccacggaggttgggccgttctaagcaaaagattcggaagtgcgcgggaatcggtgcggcggcccaatcacggctggggagcgcgggaacgagcaacagtgctcagagcaggtttagtcccatctcgtccagtcacaaaaggaacgaccaccttataaggctcggctgccacagccgtttc

SEQ ID NO: 17 - DNA, 160 bp, synthetic construct
  source 1..160: mol_type = other DNA
agaaacatggcccacggcccaatacgaagcaccgcgacgaagcccaaacagcagtccgtaggtggagcaaagcgctgggtaatacgcaaacgttttgcaccagaccggccagcgagcattgcagacaccggcttataagttcagctgcgactaccactcc

SEQ ID NO: 18 - DNA, 300 bp, synthetic construct
  source 1..300: mol_type = other DNA
tcacagctccaaaaaaagagaagaagtgtgagttacttctgtttttagcaagtgtgcgtggcgaaaagggcctgctgaacaaggagaacgaaataagtcgcggggggagcccaactgggctcccaccttgctcaggcccacgaagaagcccagtgacacgcgcgcttcagcccactgcacggcctggttggagaagcgcggagcgcggtgcggggcggggagaggctcgaggtttagtaccacatcgaaagcgcagctaacgacagaccactttataagccgagccaccctgctgctctc

SEQ ID NO: 19 - DNA, 300 bp, synthetic construct
  source 1..300: mol_type = other DNA
ttagtgtggggatagtaccagcaccagaccggttcgggcaacaaagaaagaaagtacctgttgacggtggcaaaaggaggagaaaaacacttcccaatttagtggcaaaaagcccgtgggccagccgcggccgttgggctgggcccaccacgtgcgcttattcagcccaataccgttattgagcgcgctctggagaagtggagcaacgagcgcggggaacaagcagaggaagtttagtaccacctcgagccagcaaacgcgaaacgaccaccttataaggcgctgagcttgtgctgcctc

SEQ ID NO: 20 - DNA, 300 bp, synthetic construct
  source 1..300: mol_type = other DNA
tgttcgtgaagtttttttttgggaaaaagaccgtgagcgattacaataatatcaagtgttactctgtgttgacggtgggaggagaagcaacgaggcgtagtgggcgtatcagcaaagagcttgctccgaaccagcaagaaggtgcagcaagcccactggccccgcggtcgtgagagcgcgctgcgcttgcgggatccgatcggtggagcaacagcgttgcgcttattttagtttagtcccatctcgtcccagcaagccagcttataccactttataacccgcgcgcgctgccttgtgttc

SEQ ID NO: 21 - DNA, 300 bp, synthetic construct
  source 1..300: mol_type = other DNA
gaggttccaaaagaagtgaagtgagcaacagtaggacacaattccagcccaggaccgaactcttcctgtacaaggccgggaggagctgggcgtagtggctctggactgggctctctactgctgatacggccgtaaggaaggttgctaaagttgggcgcgcaccgcgctggcgctggtcgtgtcagcccaattagcgcgcttcggtggagaactggttgcttatcgaagaaagtttagtaccatatcggccagctagtttagagcgtaccagtttataaccccgcggagccgcttaatctc

SEQ ID NO: 22 - DNA, 300 bp, synthetic construct
  source 1..300: mol_type = other DNA
ccacagctaatttcttatacaaaaagagccaggagcaacgaagcctgtagcaccgcgttgagaagcttcaaaaagaggcaaaaggcgtacagcaactgggattttgaggtgggaaagccacttttgctgggccgtaaagcaacagcccaaggccgttctaaggcccactcgctgcgctggcgaaggcaacagttcggtagcgcaacgacggagagtgtgagcgagccgaagtttagtaccacctcggctgcttagtagacgacagaccagtttataaggctgctcggctgcagctgcctc

SEQ ID NO: 23 - DNA, 300 bp, synthetic construct
  source 1..300: mol_type = other DNA
gagcactgcacacaaaggccggtttttgcaagcagcaccagcaagtgtgctattcaagtgtcaaaggcccagcacaaggcccaagcttattgtgtgtagtgaggttatcgactgggccagctgtggaaaggtggaaagcccacaaagcccagcccgtcttggctgggctgggcggcccaacagatagctcggatagtggagcgcagtaacgagcgagcaggacgcgctaagtttagtaccacctcggttgccaacgcaaacgcagaccagcttataaggctgagcctgctgagctttgtc

SEQ ID NO: 24 - DNA, 300 bp, synthetic construct
  source 1..300: mol_type = other DNA
cacgtaacagcttgcaacctttgcaacagtgcaccgcaggacacagttacagtgttcgcaataagtacgttcaaaaattctggaccgaaagcaaatacgaagcaaaagcccactaaaccagcccacggagaaggaaagcgaagaagcaagcccgtgtactatataaatccgttatactggcgcgacttcggtcggtagcgcaacgcgggaacgagcggggagggcaacgaggtttagtaccacatcgatcggcaacgcgaaagcgacccaccttataaccgagcgcgcgctgagccgttc

SEQ ID NO: 25 - DNA, 300 bp, synthetic construct
  source 1..300: mol_type = other DNA
atagattggaaacacggccagcacaatcaatcagttaccctggcccaattccgcgttgagaatagaggtgggcctggaactctttttttttaaaacgaagccctagtggcacttcgcaagaaggaacaggccgtaaggaaggcccaatacgcctccgaaggcccacgcgctgggcgctgcgctgcggagcgcgcgcttacgggaacgagcaacgcgctagggggcgtttagtttagtcccatctcggcaacgcaaagcgaaacagaccactttataagcccggaagcgaagcctgctctc

SEQ ID NO: 26 - DNA, 300 bp, synthetic construct
  source 1..300: mol_type = other DNA
gaataaaccttccttcccttcttccaaataacgatttctggttcgcaatcgaccgaggtggagagttacacagctgggaggaggcaaaagaggcttggtcgaggcgtgtgctcttcgttaaggccgccgccgaacaggcctccgaagtggaaagcccagcccacgcgctggcccaagtcagcccacttataagcaacgacgcgctaggcaacgagcgtttaaggcaactcgtttagtaccatatcgctagcgaacaatccacaagaccagtttataacgctagcttaatctgctgctctc

SEQ ID NO: 27 - DNA, 300 bp, synthetic construct
  source 1..300: mol_type = other DNA
atcaatcagagctaattttctacttacacgggcgtttttcgcagtgtgtacagaataacaacgtacagaatacaacaacaaaaagcagcacggaagcccaagcccaaggcccaattaaggaaggaaaggtttgctgaagcgtggtgacacgcgggccgttaccgcgctggcgctgcggcgctgcgctgcttcgtctcgttagcgcgcaacgagcgcgggaggcaatcagaggtttagtcccatctcggctagcgaagcgcaaaggaaccagtttataacgctgtacggaagcggaagatc

SEQ ID NO: 28 - DNA, 300 bp, synthetic construct
  source 1..300: mol_type = other DNA
tctacaatcaatcagaccttcttttttccctttgttcgcagtgcacccagaaagaatacgttcgttcaaaaaagactgggcctagtgtgcaacgacgaggcctctgagaagcccacttcctcacagagaagcgggaaggaaggtgcccacaaggtcccgagcgcggcccactctggcctcctgcggagtcagcccaatagcgcgggaactaatagagcgagcagagtttagtttagtaccacctcggctagcgagaaagaccaagcccaccttataaccgagcaccggaagcgctgcctc

SEQ ID NO: 29 - DNA, 300 bp, synthetic construct
  source 1..300: mol_type = other DNA
gagtgtgagagaatttctttttagcaccgtgagaacttcaccagaggaagatcctgttcgtacttagagtttagtcacggtcgctctgtttttttagtggggaagcccactcacagaaaggacagagaggcccacggccgcttaaggtccagccccgcgggagcgcggtgcgaggcccaatctggcgggctcggtggagcgcttacgcaagtaacgcgcgctggagtttagtttagtaccacatcgccagcaaaggctagcgcagaccactttataaggcaagctggaagctgctgcctc

SEQ ID NO: 30 - DNA, 300 bp, synthetic construct
  source 1..300: mol_type = other DNA
ctacgtacttctttttcttctacttgagcaagtgtgtgcaccgcgttttttagcgttcgcacgttcacagcccagctgggcccaatttgtttagtgtgtatcctgagccaaaaagcccaatcagcccactgggccgagtgctgggcctccgcgcgggaagtaccgcgctgcgagaaaagggcggtttgggtgtaaggcgcttaacgcgcggaggaaggtttaagcgggggcgtttagtcccatctcggccagccagcaacgcgcagaccagcttataagcttaccgagctgctttccgtc

SEQ ID NO: 31 - DNA, 300 bp, synthetic construct
  source 1..300: mol_type = other DNA
gttttcttatctacggttttatacaaaaagaggtataataacagtgcaatcgcgagatagtactttagtaatagagaaagcactcggaggagcccaacaagggcaaaaagccaaaagcccagcccaatactgggcctgggcccacagacgcgcgggcgcgggccgtgcgctgcggccccgaggctggttcctgcggagcgttgctgggagcaagcggggcaacaggaatcgtttagtaccacatcggcaacgcaaacgaccagcgaccagtttataacgctgccagagcggagcgctgtc

SEQ ID NO: 32 - DNA, 300 bp, synthetic construct
  source 1..300: mol_type = other DNA
aggttaattgaaaaagaagtgtgtgacttacaacgattacacagtacagaatacagtgtcaggccccaacggtgggagaaaaggcgtaggcctgggttagtggactgctgggcctctctgggtacggagaaagccgaagaaacaagaagaaggccgtgtaccgcgcggcgctggcgcagaaggcccaattcgcgctctagcgcggggcaacgagcggggcaacgcgctaagtttagtaccacatcggaaaccgaacagctaacgacccaccttataaggtggtaactgcagctgccactc

SEQ ID NO: 33 - DNA, 300 bp, synthetic construct
  source 1..300: mol_type = other DNA
cagagtttttatacctgtgtgtaaaatacaacttctgtgtacttgagatagcaccgtccccggcccagcaaaaattctggcaaaaaaagaggcttcccactaattgagaagaaaagaagaggcctaagaaacgcccgtgggcccacaaggcgggccgctcgagcgcggcccactttcgagcagttcgtcgaacgagagcgcggggagcgcgggaacagtggagaggactcagtttagtcccatctcgagcaaggaaacagaccaagaccactttataacgtagcctgctctccacccttc

SEQ ID NO: 34 - DNA, 300 bp, synthetic construct
  source 1..300: mol_type = other DNA
aattcttatttcttttagtaccttcttctgggggcggcgttcgcagaaagaatacgttcgcacgtagcgaaaagggccgaaaaaggaaagcctggaccaaggaaaaacacaaaaagcgtatcctggaaaggccgcttcggtttagcccgtgtgcgggggagcagcccaatacactttagcccaaattagcgcgggtcggtggaacgcggggcaacaggcggaagtttagtcgtttagtaccatatcggaaattaagagcgaacagacccactttataacgctgtagctaccaccctggtc

SEQ ID NO: 35 - DNA, 300 bp, synthetic construct
  source 1..300: mol_type = other DNA
tagtacggctgaatcgtgtctccagcagaccgggcacagtgtgtcaagcgttgacgtcaagcgttcgccgcaggcctggcccaatctgggtcaacagggcaaagccctcgcaagaaaagaaagaaagaagccagcccaatcggcccagcccacgcctcaacaacgagcccactgcggccttgcgggagcgtccttcggtcggtggagcgcgttgcggggtttagtacctcagtttagtaccatatcggcttagcaaggaacaaggaaccagtttataagctgcagctgtgctgcctggtc

SEQ ID NO: 36 - DNA, 300 bp, synthetic construct
  source 1..300: mol_type = other DNA
cgtgtgaccggcgcacttaaaacacacgggcgtgtgtcaccagaggtatatcaagcgcgcaatacgcacgtaggtccgctgtatttgttaaaaaatcagctacttgttgttgtttgctccgttgggccgcagcgaagaagatttggcgggcttcagcccagcccgcagcttgcgggcgcttccgcgagcggcccacttcggtggagcgcaacgcgcggggtttaagcgttgtttagtcccatctcggaggcaaagctagcgaaagaccaccttataaccgagcggaagcgctgcttgatc

SEQ ID NO: 37 - DNA, 300 bp, synthetic construct
  source 1..300: mol_type = other DNA
ccagcaccagcagcaccagagttcgtgaaaccttcttctgttcgtcaccagaactcttcttcttctgtgtgtgtgcaaaatctgggttacaacaagggcgtgtggcgtatcctcacaggaaagaaagcgaaccttgggccccgagcgggccggaagtaccgccacaaagcccacgaggcccacttcggttcgtctcgtcgcgcttacgcgcgcggggtttaacaacagaggtttagtaccatatcggaaacacaaagcgaagcgtaccaccttataaggaaagcgaagcgaagcgcgctc

SEQ ID NO: 38 - DNA, 300 bp, synthetic construct
  source 1..300: mol_type = other DNA
atttttaaccggttctttaacagagtgaagaagtgtcagaaagctcagtaggagccgcgagaatagtcagaggagaaccgagaagcctagtgttaaaggcttgctgggctgtgcaaagaagaaagcccacttgggcctgggcctcaacaagcccaatacgaaaggctgggcgcgctgcggggggcttgcgggcttcgtcggcgcgcttgggggcaacgagcagaggtttagtttagtaccatatcgcctcgaaaggcaaggaacgaccaccttataacgctgcagctgtgcagcttaatc

SEQ ID NO: 39 - DNA, 300 bp, synthetic construct
  source 1..300: mol_type = other DNA
gtttttttcttctttttttcttttttctacccttctacgccaggagataagtgtgttcgcacgcttcacctgtacaaaaaagcaactgggctccagcaacgagagccacaatcaacttcgttaagaagcaagaaagatctccgctagcccgtgtactcgcttccgtcgggaggcccactctggcgcgctgcggggagcggcttgcaacgcggggcgggggcgctagaacaggtttagtaccatatcgagcagttaacgcagcaacgaccactttataacccgccaaaccaccctgctctc

SEQ ID NO: 40 - DNA, 300 bp, synthetic construct
  source 1..300: mol_type = other DNA
tttatatactgttcgctgtcgcttacagagccagaagtgtgtcagcgccgcgaggtgctggtgtacaagaagagctttgttaaaaaaaggcgtatcagcccactgggcccaccttgggctaaagcttataagcggcccaaagttgggcctccgcgggctctaccgaagcccacacgggcgggagcggcccagttcgcgctgggattgcgctgagcagagcgttaagcgttgtttagtaccatatcggctgctcacgcgaaagcgacccaccttataaggcgcttaaccggaagcctggtc

SEQ ID NO: 41 - DNA, 300 bp, synthetic construct
  source 1..300: mol_type = other DNA
caaaggcacaccctcgtgaaacaaagcaacaaagaggcaacagtttttagctggattacagcaagaagcgcgcttcagctgtatctggcggcctggactgagcaatcagctgctgatttgcaaaagcggccgtgcacaagaaacaggccgtgggctgctacaactgggctggcccaccgcgggctggccagttcggtagcgcttacgagcaagcgggggcaacagagtttgtttagtaccacatcggttgctcagcgcaaacgcgaccagcttataacgctttggcaccgttgcgggatc

SEQ ID NO: 42 - DNA, 300 bp, synthetic construct
  source 1..300: mol_type = other DNA
ttgcgcttcgctgccaaaattgctgtcaacggttcggagtgcagatagtcctgtttttttgtagccgcgttcggagaactgcaaaaaaatcagctgacgaggctctgatcgaggaagaaaggccttgggccgtttcttcggtttagaagcccacgcgggctgtgggcgctgggctgcggcgctgcgctgcagtagtacccagtaacgcagtaacgcgcaaacgtttagtagtttagtaccatctcggctgccgaaagctaacgaagccactttataagcctcgcgcggaagcaccagttc

SEQ ID NO: 43 - DNA, 300 bp, synthetic construct
  source 1..300: mol_type = other DNA
aaaggagcaaaggaatcgttttcagagtttacaaattgagcgaatcagcccaagaatacgtgtcaaagacttgagcttatctggtattataaacctggtaaaggggggagaaagccacttgggcccagcctagaaagcaaggtgaggaaaggcgcggctggagagtgctggcccaatacagttcgttccgggctggggagcgcaagcgcttacgcggggcaacgagcagagtttagtaccacctcggttgctcaacgcaaaggaacccaccttataaccacttataggcgctagctgctc

SEQ ID NO: 44 - DNA, 300 bp, synthetic construct
  source 1..300: mol_type = other DNA
ggtaagtttttcttttttctttttgccgtacagtgtgtgtcaagacctgtagcaacaaaattgagcttaaaagagtttactgggctctgaacagctctgagaaagcccacggtgggctgaggcctcctcaactgggcctagaaggcccagaaggcctgggctgggcttccagtacgcgggtttgggccaggcgggaggcaacgcaacgcagccagtgcgctaaaacgtttagtttagtaccacctcggctgcgtagaaagctaacgaccagcttataacgtagccgagcctcacagcgtc

SEQ ID NO: 45 - DNA, 300 bp, synthetic construct
  source 1..300: mol_type = other DNA
tgcacttatttagtaccagcaaccaccagaggcggcaacagaaaacgttcttcaattgttgtgggctgtgttagaggagctgtgtgctctgaggctctgggctctaacaaaagctaaagaagagctgggccggcccaaagaaacaggccgtacacgcgctcggcccacgcgcttgctactggagcagtaaggcctgggctggcgcttgccggtggagagcagaggttaacagtttagtaccatctcggctgctcaagctaacgcagaccactttataagctggagctgccttgcagcgtc

SEQ ID NO: 46 - DNA, 4883 bp, Zea mays
  source 1..4883: mol_type = genomic DNA
atttatttagatgggcccttgcaaacctgtagatatcatctcttgcaacattctttctatagcaccacgaaaatgtattgcggctttgaaattataattgaattagttgtatcattttctttcaccgatgcgttaaattcaaaattaagtgttatatttcttcataatttgttaaatatatagaccctataatccaccattatttactataatagcatacattaacattggttttagcctacactacgacactcgaggcattgaattttcctctatcaaagaattatatgtgtagtagtattgttcttgacaaaaagggggattaaaattaaactaccaatattgatacttatcttatcacatccatgaatacaatcaacactcttacaaaagataagatacaagattaaaaagtaccatgataatacattaagattattagcaatgcattaaattaaataaatgtgcaagtgaatcatgattttagttttatctattttacttttaaaatatgatattctctgactacttctaagcataaatgtgattctaagtcatgaccgatcgtgcttattcagaaaaatgaaggagacacagatttctataaaaaaaggttgtcatgggactattgggtcaaccatcttattcatttgggaaaataagtttagaacacatcaacccattttagatgttgagtttggccctaatggtccattgaccttacttttgtgggttgacatagaccatctatcccaagttattgttgtgtcacattccctgatatcatgaatctatattttagctttccgttttcatatttttagtcgttacatattttttatccgcgtactagattaaaactctagttgttgcaatacattttgttcatttttttctatttcttctttactaacaacatattctagttcctagctacattcttaagtaccatagtgctataaacattttttatcctacattattccacttaagaaattgaattttctgcataaaaaaattatatgtccagtagtgttgtcttataaaagcataaagtgattaaaattaaaaccattattgatatcttatttttcaaaaaaaatataagcttatagaaagtgaattaatttcatggtaaattaatatagtttaaattgaattattagtgttattactatgtttattatcaatgaaacatttttcatggttgatataacttagtgttacttattttagtattttttatataattctagttaacttttagtttttgatttaaaaaaacgagaattgtgtccttttgtggagtgagtataaagaaagtaatatctgttcatcataatttggttttttaaggtacgtgaaacttgctttatatttggactcaagctatgtctaaatacatagtaaaaaagcaatatttctagaaaagacaaaacatcttataatttagaatcaaggaaatatatagattttatgtgcagtgagaagccatttacaatggaacgttcaacgttgggccaatagatattttgcgatatgatgatgggcatatttttgcatggttgtccctccactagctatagtttgatgatacgatacgctgcacacaccattgggttgtaccatgttagtgtagcaacagtagaaacccaattgtggccgtgaaccatgataatactaggtagagtgctagctagaggtttcaggctattgatgcgtgaattaaactttctgttgtgttgcgaggaaacgagtattgtgaaatatttgaaacggtttttttgtgaaagatttgaaacggtatttttgttgtgaaataaagatcaaggctaaataaattcaaactaataaaacatattaattgacggcctgaagcccccgcccccatggccccatgccatagcatcaggtcccacatgacatgaggccgcgcctccctctatgttggctccctgccttcgccgttgtcgtcgctcccgaactccctctcctcccctgttacaaatacccccacccgcccggacagcttccctgcacactcgcagctcgcacatctcatggtgtcctaagaacggcaagagccagctctgcctagcagcagcgcacagccacatccatggacgccagcccgaccccaccgctccccctccgcgccccaactcccagcattgacctccccgctggcaaggacagggccgacgcggcggctaacaaggccgcggctgtgttcgacctgcgccgggagcccaagatcccggagccattcctgtggccgcacgaagaggcgcggccgacctcggccgcggagctggaggtgccggtggtggacgtgggcgtgctgcgcaatggcgacggcgcggggctccgccgcgccgcggcgcaagtggcggcggcgtgcgcgacgcacgggttcttccaggtgtgcgggcacggcgtggacgcggcgctggggcgcgccgcgctggacggcgccagcgacttcttccggctgccgctggctgagaagcagcgggcccggcgcgtccccggcaccgtgtccgggtacacgagcgcgcacgccgaccggttcgcgtccaagctcccctggaaggagaccctgtccttcggcttccacgacggcgccgcggcgcccgtcgtcgtggactacttcaccggcaccctcggccaagatttcgagccagtggggtgagtaaagaagaagatggcgccgaatttacatttataagtaggaccagcagaagcccctgcccctgggggccttagcattgcattcgactgatgaatacgcatggcaggcgggtgtaccagaggtactgcgaggagatgaaggagctgtcgctgacgatcatggagctgctggagctgagcctgggcgtggagcgcggctactaccgggagttcttcgaggacagccgctccatcatgcggtgcaactactacccgccgtgcccggtgccggagcgcacgctgggcacgggcccgcactgcgaccccacggcgctgaccatcctcctgcaggacgacgtcggcgggctggaggtcctggtggacggcgagtggcgccccgtccggcccgtcccaggcgccatggtcatcaacatcggcgacaccttcatggtaacgaacgaaagcgccggctcctctgcttttcttggcctctttgtccctgccctgtgctgctgtgcatattcattcattcagttctctgtggggttttttttttgtttaatttttttttgggatcgtatccagtgcacaagggccacgccgtgcacaaatgcacaaaacgaaatctggccgtccattttccatccaacgacatgccggcgcggggggtttttcacaaaacagactcggaagctacgggggtcgcggaagggtcatctgcatatttacgacggccgttggatggaaaatggacggccagatttcgttttgtgtatttgtgcacggcgtggcccttgtgcactggatacgatcccatttttttttttgccccgaatcctagtggacctaactggacagattacagcacgcacacgtaggcatgtcatgtagcagcactgcagtcgggtgcagtccagtccagtcctgtccagccgcgacactgtagtacatagcgatgcaacggagacacgcgttggagttggttccatctcttctcggcggccgtgccgaggcttccgcggggaagctgcgacaacagaacggaccgccgggggtgggcaggcagcaagctccctgttggcttgtgccgttgcgcagcggcgggtaccggacaacgctttcggcggcgcgcggcctcgtcggcttcccctgtttttgatgccgcctctcggtgtccggggaccgggaggatcgatggggcccgtgccgtctgatccgccacgcgagcggtcctatgcgatgcgccgcacgagcgcgggggggccgtggaacagtacacagctgggtcactcactcactcatcccgctggttgtggctgcttggttgcaacttggctcggctgtctgtctgttgcccccgccgcgttttctagccgtttccgctttgctcgcggtttcgctggcgatccggcacgcggcgcccacacccggggctggccccttggccgagtgggtggcaggcacttgcatgcatccggccggtttcccgcgaccaagctggcccgccgcaacaatgagagtgagacgagactttgtgtcagtgtgtgtatgtacatgtatgtctgcgcgacagccctaccgtccgacacgatgattcttgtgcactgtactgtactgtactaactccccccaccccctccggtatgtaacgcatgccatatgcaggcgctgtccaacgggcggtacaagagctgcctgcaccgcgcggtggtgaaccggcggcaggagcggcaatcgctggccttcttcctgtgcccgcgcgaggaccgggtggtgcgcccgccggccagcgccgcgccgcggcagtacccggacttcacctgggccgacctcatgcgcttcacgcagcgccactaccgcgccgacacccgcacgctggacgccttcacccgctggctctcccacggcccggcggcggcggctccctgcacctaacgagccggccgtctctttcgccggggcccgcgcggggttcgcccacgtggtgatcaggtggcagacatgtggcccacgggccccgcgccgccttccccatttttggacgaccctactgctactactactagtgtacatatgcaaaaaaatacatatatatataggtactttctctaatatttttatatataagcaaggcggcctggtgttcttttctttgttttgtcgacaactgtttgatcccatcctatggacgatggatagttcaatgtttgtac

SEQ ID NO: 47 - DNA, 3955 bp, Zea mays
  source 1..3955: mol_type = genomic DNA
ataaaaagttgtagatcttaaaaagttattaaactttatagttagttagcaactttttcgtttaaattcgtttagtgctacaaacaatcgatttatattcggtttgttttaatatgcggacaacaaacctataatctaaacataagttaaagttagtgatgggtaaagtggtagagaagtgaggttagagtttcgattcgcgaaaggtcctacaacaaacctaaaatctttttttgctatttattttaaaacatggtttatatttccaaaaaaatgtattggcggtttttaactcaggcgccaacaaaaattgatttccacggcagctgccaccgtcgttatcgctgaccaacccggctggtcgcctgtctgctccatccatgcatgttacaactatgcagatgcagccgaaacaaacactggctagaaaggcagccaacgggcctactgtcattcgctccggcatcctactggtgggcccagttgcaccggccgatgaccagttcatcatttttctcgacgaatttgtgcacagatcgaatttactaaaaattcttcgcacgtggcaaaaccaggggaaaaatcgagaactagtcgggtttttttatattccctgatagaatagtccctgctattcatccatgaaaaccaaacacgtacactacgtcaccgtcatggatgggagcgagtgaactgatgattttttccccaccccgcacgcaacagcatgggtgacaacaaccactcccgctgcggttgggcgagcacatctctacgcacttgacactcacgcaaacctaacgcatactagagtaatcatcgccaccaactatcggcgacagaaacgatgggccccgcttctcttaatcacggtgcttgaattagtgcgcgcatagtagtgaaaataatagtgaaaaataagcagtgcgtgttttggtgtggtggttggtgagccgtccggcccaataaaaaccctcgcaccacctcgtccctcttcgtcgcatcgcacgccatcagctagcagctagcgcgctcctcgagcccagcagagaaaggccggcagcgctaatcgtaatagccatgggctccaccgccggcgacgtggccgcggtggtggacgaggaggcgtgcatgtacgcgatgcagctggcgtcgtcgtccatcctgcccatgacgctgaagaacgccatcgagctgggcctgctggaggtgctgcagaaggaggccggcggcggcaaggcggcgctggcgcccgaggaggtggtggcgcggatgcccgcggcgcccggcgaccccgccgccgcggcggccatggtggaccgcatgctccgcctgctcgcctcctacgacgtcgtccggtgccagatggaggaccgggacggccggtacgagcgccgctactccgccgcgcccgtctgcaagtggctcacccccaacgaggacggcgtgtccatggccgccctcgcgctcatgaaccaggacaaggtcctcatggagagctggtgagtagtagccgcatcgcatcaaccaccttctacctatctatatccatcacttgttgctgctggcgtgcgcggcatgcatgatgacgagctcgctcatcattggtgctactagtgatttatttcgtccagtaaaattaattaaggtgcgctgctactctactggctgcggctagcacaaggctggaaatagttgttacttgttatacacgatataatatttctctagaacaaaaagatttttttttttataaaaagcaagcaagaaagaaagtgagtgacttcatgtttttcctaaaaaaaagttaggagtgggatggaaaagtcagcaaggaccacttgtttgttgtccactatccatccagtgggtgagacttttttgcgagacggagcactatattattggccgagtcctttttctgtatccgcaaaacggcagccgtcgatcgccggacggatcgacggctcacatgagtgtcgagtccaattccaaccacgagggcggcaaggaaaaccatccgtgctggtctggactttttgccaaactccattcagccattcgccgactgaaggtgaatcttcagacagccagattgtttggtgtctagtgtgtgcgaagatggcgtagaaaagactgagagacagttggctcacacagacaagtgacaactgactatagtatctgcctgcctggctgatgctgatagagatggggactcttgtcctgtctgtttcttgtatgcgctgatctgattctgatcactgccactctgccaggtactatctcaaggacgcggtgctggacggcggcatcccgttcaacaaggcgtacgggatgacggcgttcgagtaccacgggacggacccgcgcttcaaccgcgtgttcaacgagggcatgaagaaccactcggtgatcatcaccaagaagctgctggacttctacacgggcttcgagggcgtgtcgacgctggtggacgtgggcggcggcgtgggcgccacgctgcacgccatcacgtcccgccacccgcacatctccggggtcaacttcgacctgccgcacgtcatctccgaggcgccgccgttccccggcgtgcgccacgtgggcggggacatgttcgcgtccgtgcccgccggcgacgccatcctcatgaagtggatcctccacgactggagcgacgcgcactgcgccacgctgctcaagaactgctacgacgcgctgccggaaaatggcaaggtcatcgtcgtcgagtgcgtgctgccggtcaacacggaggccacccccaaggcgcagggcgtgttccacgtcgacatgatcatgctcgcgcacaacccaggcggcaaggagcggtacgagcgcgagttccgcgagctcgccaagggcgccggcttctccgggttcaaggccacctacatctacgccaacgcctgggccatcgagttcatcaagtgaatacggctaccaccgtcgccgcgatgagatgcatggctgccacatgcatgcttgcttgcttggtcctcgtatcgtacgtcgccgtcgtcgtcttcttctggttgcgctgctaccttgctgctctcgccctcgcgtatgcatgtacttttgcttaattttctttcttcatatcatgcactctggctggcctagactgcccccgatctatgctggccggtacgtcttgtcgagctcttgcatgtcgtggattctaaattcttcttctgcgtcgaattgtctctgccatgtgcgagtaataacaatcaaggttatacttaccatacaattacatggcggtttaattgctctcttttaatttggtgaagaatcgaatcgattctactgaattcttctctgtcgtttaatgaaatttcctttgtcttttggattatataatataacacagacactcacaaagacactcacaacgtgcgcacactcattcctataaacgcacacgcgcaaatcctactcctatcaagtatcaacactttcaagacttgaactggtcataccggagaaaatggactttcgttcgaggacataattacgtgttgctttagaaccaataccaactcaatcatcaacatcactttttaactgttagtatcggtttgtgtcttcaataagtactaatgttaagtcattaacaccggttgaatacaccaactgccgaagattgttcgacagtgaatatacgaggtatgtctatgaatgaaaacggacggaaacggtcggaaaaatcaatctcccatttcggtttccattttttatcaaaaacaggagcgggagcgatatagcaagaaatagaaaaggtagtgggatatacggttacacgaaaacggattaaaacaaacggatatgcaacgaaaatggatgctaatcggaaactttggctggaatatgcaatattttataatcactcaacgactacaatattaagagtttagaatactataaatca

SEQ ID NO: 48 - DNA, 36 bp, synthetic construct
  source 1..36: mol_type = other DNA
atggcgggatctaagaagagaagaattaaacaagat

SEQ ID NO: 49 - DNA, 33 bp, synthetic construct
  source 1..33: mol_type = other DNA
ggatctaagaagagaagaattaaacaagattga